(19) United States
(12) Patent Application Publication
(10) Pub. No.: US 2010/0063302 A1
(43) Pub. Date: Mar. 11, 2010

(54) CYCLIC SULFONIUM SALT, METHOD FOR PRODUCTION OF CYCLIC SULFONIUM SALT, AND GLYCOSIDASE INHIBITOR

(75) Inventors: Osamu Muraoka, Higashi-Osaka (JP); Genzoh Tanabe, Higashi-Osaka (JP)

Correspondence Address: BIRCH STEWART KOLASCH & BIRCH, PO BOX 747, FALLS CHURCH, VA, 22040-0747, US

(73) Assignees: Takano Co., Ltd., Kamiina-gun, Nagano (JP); Kinki University, Higashi-Osaka, Odaka (JP)

(21) Appl. No.: 12/522,111

(22) PCT Filed: Jan. 4, 2008
(86) PCT No.: PCT/JP2008/050223

§ 371 Date: Aug. 28, 2009

Related U.S. Application Data
(60) Provisional application No. 60/883,296, filed on Jan. 3, 2007.

(51) Int. Cl. C07D 333/32; C07D 327/10

(52) U.S. Cl. 549/18; 549/66

(57) ABSTRACT

Disclosed are: kotalanol which has an inhibitory activity on a glucosidase; a method for producing kotalanol or a cyclic sulfonium salt which is an analogue to kotalanol by an organic synthesis technique; a cyclic sulfonium salt produced by the method; a glucosidase inhibitor comprising the compound; an anti-diabetic agent or an anti-diabetic food comprising the glucosidase inhibitor. A sulfonium compound including kotalanol can be produced by coupling a thio-sugar synthesized from D-xylose (e.g., a compound having a cyclic structure composed of 4 carbon atoms and one sulfur atom, such as 1,4-dideoxy-1,4-epithio-D-arabinitol) with a heptitol cyclic sulfate ester having a protected hydroxyl group and synthesized from a pentose (D-xylose, D-ribose, D-arabinose, D-lyxose, L-xylose, L-ribose, L-arabinose or L-lyxose) to produce a cyclic sulfonium salt having a protected hydroxyl group, and then deprotecting the hydroxyl group.

### TECHNICAL FIELD

The present invention relates to a cyclic sulfonium salt, a method for the preparation of the cyclic sulfonium salt, and a glycosidase inhibitor. More particularly, the present invention also relates to a cyclic sulfonium salt of, in particular, kotalanol and an analogue thereof and a method for the preparation thereof, and the cyclic sulfonium salt of kotalanol and the analogue thereof, prepared by the above method. Further, the present invention relates to a glycosidase inhibitor using the same.

### BACKGROUND TECHNOLOGY

The digestion and absorption of sugars in the digestive intestine or the like can be inhibited by using a substance inhibiting the sugar-decomposing activity of glycosidase acting as a sugar-hydrolyzing enzyme, that is, a glycosidase inhibitor. Therefore, utility of the glycosidase inhibitor is expected as an agent for treating or preventing diabetes. As such a compound to be used for the glycosidase inhibitor, there is known a cyclic sulfonium salt (a thia-cyclopentane derivative) having a trivalent sulfur atom.

A cyclic sulfonium salt having a glycosidase-inhibiting activity, as represented by the following chemical formula (5), is disclosed, for example, in Claim 8 of Japanese Patent Publication No. 2002-179,673 A1 (Patent Publication No. 1); Tetrahedron Letters, Vol. 41, No. 34, pp. 6615-6618 (2000) (Non-Patent Publication No. 1); and Journal of Organic Chemistry, Vol. 66, No. 7, pp. 2312-2317 (2001) (Non-Patent Publication No. 2):

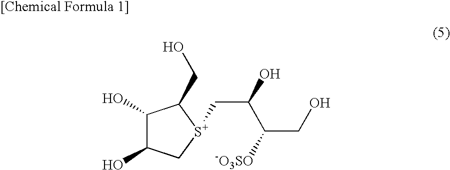

It is also disclosed, for example, in Tetrahedron Letters, Vol. 38, No. 48, pp. 8367-8370 (1997) (Non-Patent Publication No 3) and Bioorganic Medicinal Chemistry, Vol. 10, No. 5, pp. 1547-1554 (2002) (Non-Patent Publication No. 4) that salacinol contained as a pharmacologically essential substance in a medicinal plant, i.e., Salacia rediclata or Salacia oblonga, which has been used in India as a traditional medicine, is a strong glycosidase inhibitor. Moreover, the cyclic sulfonium salt as represented by the above chemical formula (5) has a structure similar to the salacinol and a similar activity of inhibiting glycosidase. For example, Japanese Patent Publication No. 2002-51,735 A1 (Patent Publication No. 2) discloses an anti-diabetic food characterized by containing salacinol.

On the other hand, kotalanol as represented by chemical formula (6) below, like salacinol, is also a glycosidase inhibitor contained in a medicinal plant, i.e., Salacia reticlata or Salacia oblonga. It is disclosed in Chemical & Pharmaceutical Bulletin, Vol. 46, No. 3, pp. 1339-1340 (1998) (Non-Patent Publication No. 5) that kotalanol has the activity for inhibiting maltase and saccharase stronger than that of salacinol. Kotalanol, however, is extremely lower in the yield of isolation than salacinol and the isolation yield of kotalanol from Salacia reticlata is at the rate as low as 0.0002% compared with the isolation yield of salacinol at the rate as much as 0.025%.

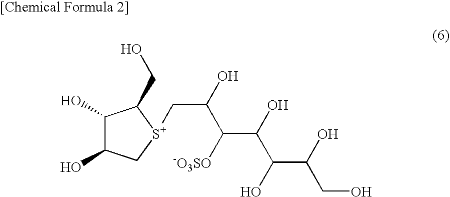

An extremely large number of people necessitate diabetes-preventive agents or diabetes-treating agents, and the number of such people in Japan may amount to more than approximately 10% of the entire population of the Japanese people. As it is difficult, to supply such a large number of people with a product purified from a naturally occurring medicinal plant, it is expected to prepare kotalanol or a kotalanol agent having a glycosidase inhibitory activity stronger than salacinol or a pharmacological activity as strong as kotalanol by an organic synthesis from raw materials readily available and as a consequence in order to allow them to be supplied readily.

In order to comply with this, it is needed to clarify the stereochemistry of kotalanol as well as chemically synthesize kotalanol with the stereochemistry retained as it is in a naturally occurring form.

As a result of research by these inventors et al., although it is indicated that kotalanol can be represented by the chemical formula (6) as disclosed above (Non-Patent Publication No. 5), the stereochemistry of a heptitol side chain moiety having a sulfuric acid anion on the 3-valent sulfur atom of kotalanol and sulfur atoms is not yet known. Further, as this heptitol side chain moiety has five asymmetric carbon atoms, it is considered to have 32 kinds of isomers.

Therefore, we have attempted to clarify the stereochemistry of the side chain moiety of kotalanol as well as to provide kotalanol analogues by preparing 32 kinds of cyclic sulfate esters of a heptitol with the protected hydroxy group, subjecting them to coupling with a thiosugar having as a skeleton a cyclic structure composed of four carbon atoms and one sulfur atom, and then deprotecting the protective group for the hydroxy group of the resulting compounds.
[Patent Publication No. 1] Japanese Patent Publication No. 2002-179,673 A1 (Claim 8)
[Patent Publication No. 2] Japanese Patent Publication No. 2002-51,735 A1 (See [0008] etc.)
[Non-Patent Publication No. 1] Tetrahedron Letters, Vol. 41, No. 34, pp. 6615-6618 (2000)
[Non-Patent Publication No. 2] Journal of Organic Chemistry, Vol. 66, No. 7, pp. 2312-2317 (2001)
[Non-Patent Publication No 3] Tetrahedron Letters, Vol. 38, No. 48, pp. 8367-8370 (1997)
[Non-Patent Publication No. 4] Bioorganic Medicinal Chemistry, Vol. 10, No. 5, pp. 1547-1554 (2002)
[Non-Patent Publication No. 5] Chemical & Pharmaceutical Bulletin, Vol. 46, No. 3, pp. 1339-1340 (1998)

### DISCLOSURE OF INVENTION

The objects of the present invention are to elucidate the stereochemistry of a side chain moiety of a heptitol having a sulfuric acid anion on the trivalent sulfur atom of kotalanol and to provide a method for the preparation of a cyclic sulfonium salt having glycosidase-inhibiting effects as high as or higher than kotalanol by means of a chemical synthesis as well as the cyclic sulfonium salt to be prepared by the above method.

More specifically, the major object of the present invention is to provide a cyclic sulfonium salt which may be represented by the chemical formula (1) as shown below and assume a particular stereochemistry as will be described below:

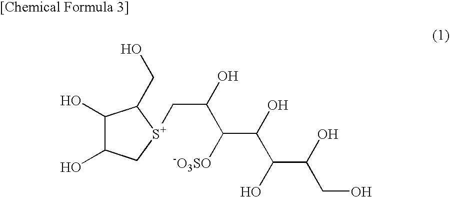

The present invention has another object to provide a method for the production of the cyclic sulfonium salt, which comprises a synthesizing step for synthesizing a cyclic sulfate ester (i.e., cyclosulfate) of a heptitol with the protected hydroxy group from a pentose or a derivative thereof; a coupling step for coupling the resulting hydroxy group-protected heptitol cyclosulfate with a thiosugar to yield a cyclic sulfonium salt with the protected hydroxy group; and a deprotecting step for deprotecting the protective group for the hydroxy group of the resulting hydroxy group-protected cyclic sulfonium salt leading to the cyclic sulfonium salt.

The present invention also has the object to provide the hydroxy group-protected heptitol cyclosulfate as represented by the general formula (2):

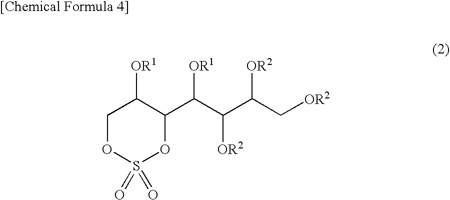

(wherein R¹ and R² are each hydrogen atom or a protective group for hydroxy group, in which the protective group comprises a cyclic acetal-protective group selected from —C(CH₃)₂—, —CH(CH₃)— and —CHAr— (wherein Ar is a phenyl group or a substituted phenyl group), an ether-type protective group comprising an alkoxyalkyl group as represented by —CH₂OR³ (wherein R³ is —CH₂OCH₃ or —CH₂CH₂OCH₃) or a silyl ether-type protective group as represented by SiR⁴₃ or SiR⁴₂R⁵ (wherein R⁴ and R⁵ are each an alkyl group as represented by —CH₃ or —C(CH₃)₃ or an aryl group as represented by —Ph).

The present invention has a further object to provide a method for the production of the cyclic sulfonium salt with the protected hydroxy group, which comprises the synthesizing step for synthesizing the hydroxy group-protected heptitol cyclosulfate, for example, from a pentose or a derivative thereof; and the coupling step for coupling the resulting hydroxy group-protected heptitol cyclosulfate with the thiosugar, thereby resulting in the formation of the hydroxy group-protected cyclic sulfonium salt.

The present invention has a still further object to provide a glycosidase inhibitor using the cyclic sulfonium salt (1) or an anti-diabetic agent or an anti-diabetic food containing the glycosidase inhibitor.

In order to achieve the above objects, the present invention provides the cyclic sulfonium salt which may be represented below by the general formula (1) and assume a particular stereochemistry:

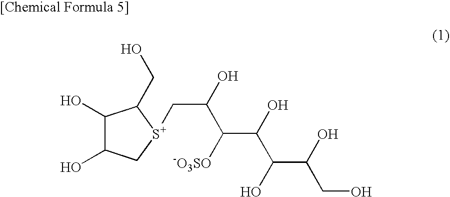

The present invention in accordance with the preferred embodiment provides the cyclic sulfonium salt which has a stereochemistry as represented below by the formula (6):

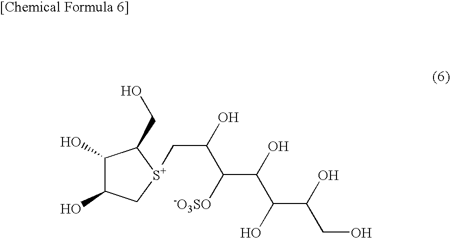

The cyclic sulfonium salt of the present invention has a particular stereochemistry structure in a side chain thereof at the positions of five asymmetrical carbons of the heptyl group.

The present invention provides the heptitol cyclosulfate with the protected hydroxy group as represented by the general formula (2):

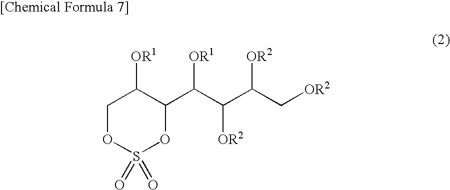

(wherein R¹ and R² are each a hydrogen atom or a protective group for hydroxy group, in which the protective group comprises a cyclic acetal-protective group selected from —C(CH₃)₂—, —CH(CH₃)— and —CHAr— (wherein Ar is a phenyl group or a substituted phenyl group), an ether-type protective group comprising an alkoxyalkyl group as represented by —CH₂OR³ (wherein R³ is —CH₂OCH₃ or —CH₂CH₂OCH₃) or a silyl ether-type protective group as represented by SiR⁴₃ or SiR⁴₂R⁵ (wherein R⁴ and R⁵ are each an alkyl group as represented by —CH₃ or —C(CH₃)₃ or an aryl group as represented by —Ph).

The present invention also provides a method for the production of the cyclic sulfate ester of the heptitol, which comprises the synethesizing step for synthesizing the hydroxy group-protected heptitol cyclosulfate as represented by the general formula (2) above, for example, from a pentose selected from D-xylose, D-ribose, D-arabinose, D-lyxose, L-xylose, L-ribose, L-arabinose and L-lyxose and a derivative thereof.

The present invention according to the preferred embodiment also provides the method for the production of the hydroxy group-protected heptitol cyclosulfate as represented by the general formula (2) from the pentose selected from, for example, D-xylose, D-ribose, D-arabinose, D-lyxose, L-xylose, L-ribose, L-arabinose and L-lyxose and its derivative, as represented below by formula (3) or (4):

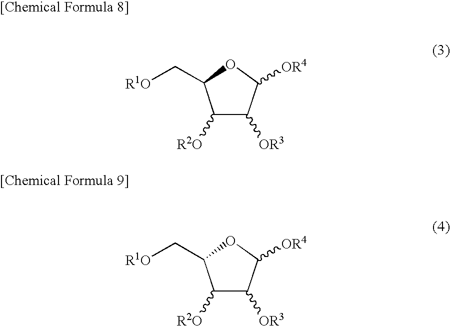

(wherein R⁴ is hydrogen atom or a hydroxy group-protective group comprising a cyclic acetal-protective group selected from —C(CH₃)₂—, —CH(CH₃)— and —CHAr— (wherein Ar is a phenyl group or a substituted phenyl group), an ether-type protective group comprising an alkoxyalkyl group as represented by —CH₂OR³ (wherein R³ is —CH₂OCH₃ or —CH₂CH₂OCH₃), or a silyl ether-type protective group as represented by SiR⁴₃ or SiR⁴₂R⁵ (wherein R⁴ and R⁵ are each an alkyl group as represented by —CH₃ or —C(CH₃)₃ or an aryl group as represented by —Ph).

Further, the present invention provides the method for the production of the cyclic sulfonium salt with the protected hydroxy group, which comprises the coupling reaction of the hydroxy group-protected heptitol cyclosulfate (2) obtained by the above step with the thiosugar as represented by the general formula (7′):

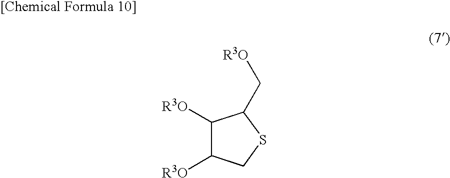

(wherein R³ is hydrogen atom or a hydroxy group-protective group comprising a cyclic acetal-protective group selected from —C(CH₃)₂—, —CH(CH₃)— and —CHAr— (wherein Ar is a phenyl group or a substituted phenyl group), an ether-type protective group comprising an alkoxyalkyl group as represented by —CH₂OR³ (wherein R³ is —CH₂OCH₃ or —CH₂CH₂OCH₃), or a silyl ether-type protective group as represented by SiR⁵₃ or SiR⁵₂R⁶ (wherein R⁵ and R⁶ are each an alkyl group as represented by —CH₃ or —C(CH₃)₃ or an aryl group as represented by —Ph),

thereby resulting in the formation of the hydroxy group-protected cyclic sulfonium salt as represented by the general formula (8′):

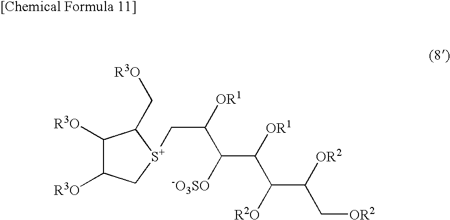

(wherein R¹, R² and R³ have each the same meaning as above).

Furthermore, the present invention provides the method for the production of the cyclic sulfonium salt (1), which comprises the step of deprotecting the protective group of the hydroxy group-protected cyclic sulfonium salt (8′) obtained by the above coupling reaction.

Moreover, the present invention provides the method for the production of the cyclic sulfonium salt in which the thiosugar (7′) to be used for the above coupling reaction is synthesized from D-xylose or D-arabinose.

In addition, the present invention provides a glycosidase inhibitor using the cyclic sulfonium salt (1) or an anti-diabetes agent or an anti-diabetes food containing the glycosidase inhibitor.

### MODES FOR CARRYING OUT THE INVENTION

The cyclic sulfonium salt according to the present invention may be represented by the general formula (1) having a specific stereochemistry:

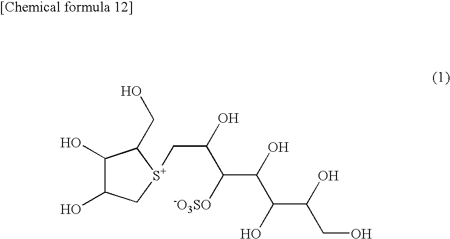

More specifically, the cyclic sulfonium salt of the present invention may be represented by the general formula (6) as having the stereochemistry as follows:

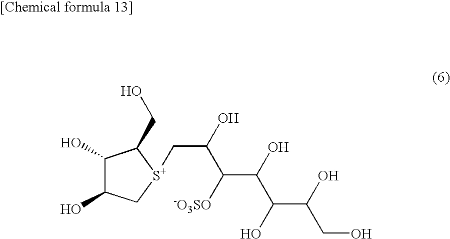

It is to be noted herein, however, that the present invention is to be interpreted as encompassing the stereochemistry as represented above unless otherwise stated specifically, although a description regarding the stereochemistry at the positions of the five asymmetrical carbons of the heptyl group is omitted for brevity of explanation.

In accordance with the present invention, the cyclic sulfonium salt as represented by the general formulae (1) and (6) can be prepared from a pentose selected, for example, from D-xylose, D-ribose, D-arabinose, D-lyxose, L-xylose, L-ribose, L-arabinose and L-lyxose and a derivative thereof by the synthesis step for synthesizing the hydroxy group-protected heptitol cyclosulfate as represented by the general formula (2):

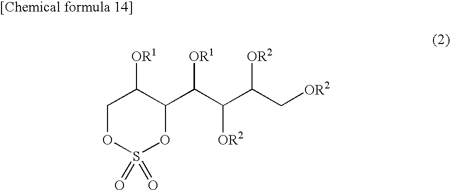

(wherein R¹ and R² are each hydrogen atom or a hydroxy group-protective group comprising a cyclic acetal-protective group selected from —C(CH₃)₂—, —CH(CH₃)— and —CHAr— (wherein Ar is a phenyl group or a substituted phenyl group), an ether-type protective group comprising an alkoxyalkyl group as represented by —CH₂OR³ (wherein R³ is —CH₂OCH₃ or —CH₂CH₂OCH₃), or a silyl ether-type protective group as represented by SiR⁴₃ or SiR⁴₂R⁵ (wherein R⁴ and R⁵ are each an alkyl group as represented by —CH₃ or —C(CH₃)₃ or an aryl group as represented by —Ph);

and the coupling step for coupling the resulting hydroxy group-protected heptitol cyclosulfate (2) with the thiosugar as represented by general formula (7′):

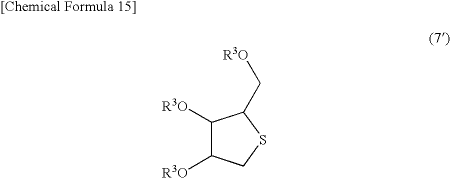

(wherein R³ is hydrogen atom or a hydroxy group-protective group comprising a cyclic acetal-protective group selected from —C(CH₃)₂—, —CH(CH₃)— and —CHAr— (wherein Ar is a phenyl group or a substituted phenyl group), an ether-type protective group comprising an alkoxyalkyl group as represented by —CH₂OR³ (wherein R³ is —CH₂OCH₃ or —CH₂CH₂OCH₃), or a silyl ether-type protective group as represented by SiR⁵₃ or SiR⁵₂R⁶ (wherein R⁴ and R⁵ are each an alkyl group as represented by —CH₃ or —C(CH₃)₃ or an aryl group as represented by —Ph),

thereby resulting in the formation of the hydroxy group-protected cyclic sulfonium salt as represented by general formula (8′):

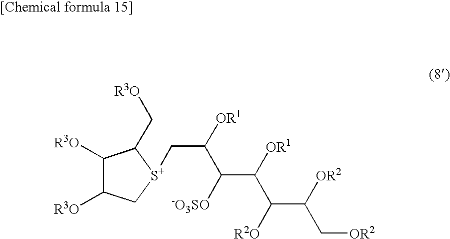

and the deprotection step for deprotecting the protective group of the resulting hydroxy group-protected cyclic sulfonium salt.

Specifically, the method for the production of the cyclic sulfonium salt according to the present invention may be represented below by the following chemical scheme (1):

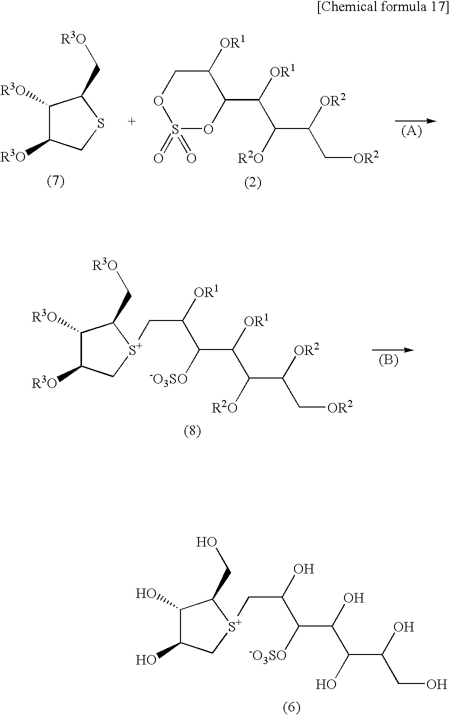

As illustrated by the above general chemical scheme (1), the method for the production of the cyclic sulfonium salt according to the present invention may comprise the coupling step (A) for the preparation of the hydroxy group-protected cyclic sulfonium salt (8) by coupling the hydroxy group-protected heptitol cyclosulfate (2) with the thiosugar (7) having as a skeleton a cyclic structure composed of four carbon atoms and one sulfur atom, obtained from the pentose selected from, for example, D-xylose, D-ribose, D-arabinose, D-lyxose, L-xylose, L-ribose, L-arabinose or L-lyxose or the derivative thereof; and the deprotection step (B) for the deprotection of the protective group of the hydroxy group-protected cyclic sulfonium salt (8), thereby leading to the cyclic sulfonium salt (6).

More specifically, the cyclic sulfonium salt according to the present invention may be prepared by a series of the steps as represented below by the following general chemical scheme (2a) or (2b), which comprise the step for synthesizing the hydroxy group-protected heptitol cyclosulfate (2) from the pentose or the derivative thereof; the coupling step (C) for coupling the hydroxy group-protected heptitol cyclosulfate (2) with the thiosugar (7) having as a skeleton a cyclic structure composed of four carbon atoms and one sulfur atom thereby leading to the formation of the hydroxy group-protected cyclic sulfonium salt; and the deprotection step (D) for deprotecting the protective group for the hydroxyl group of the cyclic sulfonium salt with the protected hydroxy group, thereby resulting in the formation of the cyclic sulfonium salt (6):

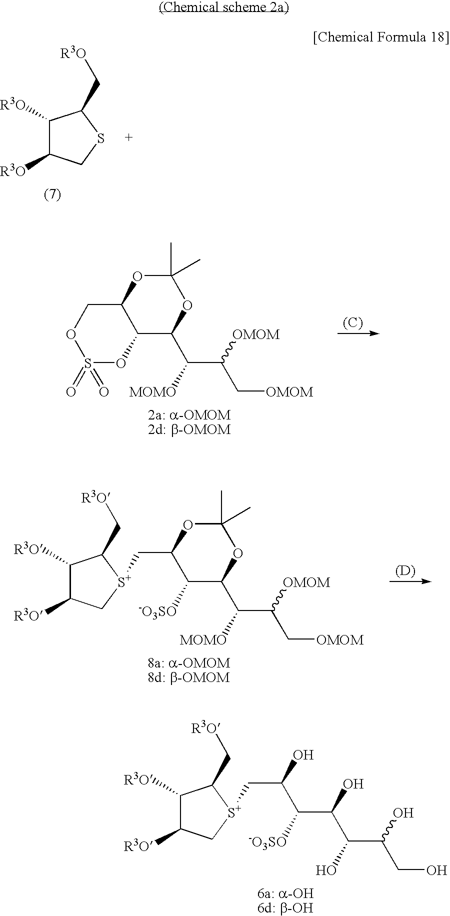

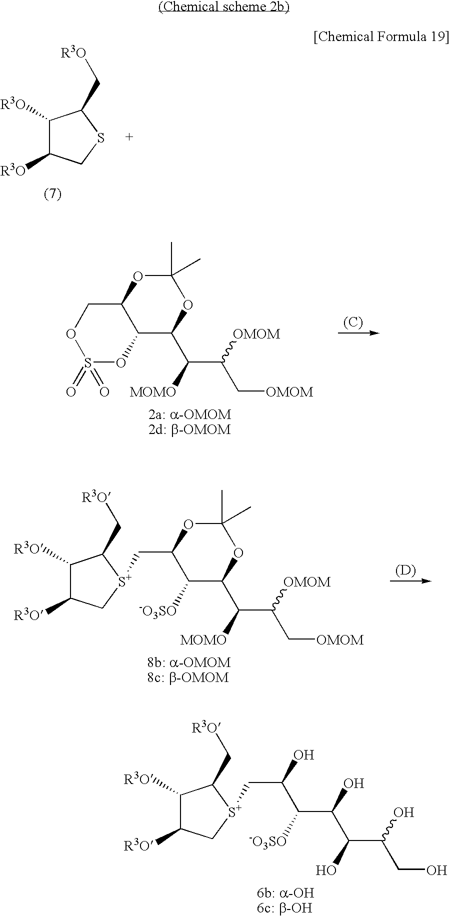

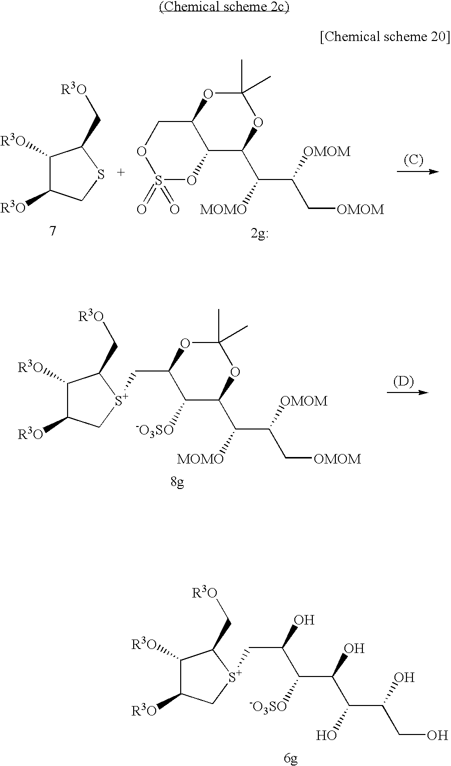

(wherein OMOM means a protective group and R³ has the same meaning as above).

In other words, the hydroxy group-protected cyclic sulfonium salt (8) to be used for the present invention may be prepared by the coupling step (C) for coupling the hydroxy group-protected heptitol cyclosulfate (2) with the thiosugar (7) to thereby yield the hydroxy group-protected cyclic sulfonium salt. It is provided, however, that the protective group for the hydroxy group-protected heptitol cyclosulfate may preferably be isopropylidene group or methoxymethyl group (MOM).

The hydroxy group-protected cyclic sulfonium salt (8) obtained by the above coupling step (C) is then followed by the deprotection step (D) for the deprotection of the protective group of the resulting compound, thereby leading to the cyclic sulfonium salt (6).

As specific examples, the processes for the synthesis of kotalanol analogues may be shown herein by chemical scheme (3a), (3b) or (3c). It is to be understood, however, that this synthesis process is solely illustrative of the present invention and the stereochemistry of the kotalanol analogues to be prepared by the present invention is not limited by the stereochemistry of the cyclic sulfate ester of the heptitol as represented below by the chemical scheme (6):

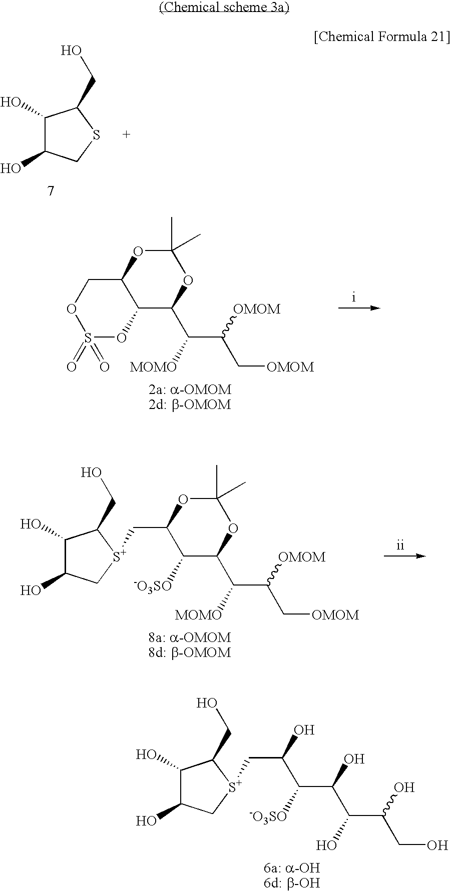

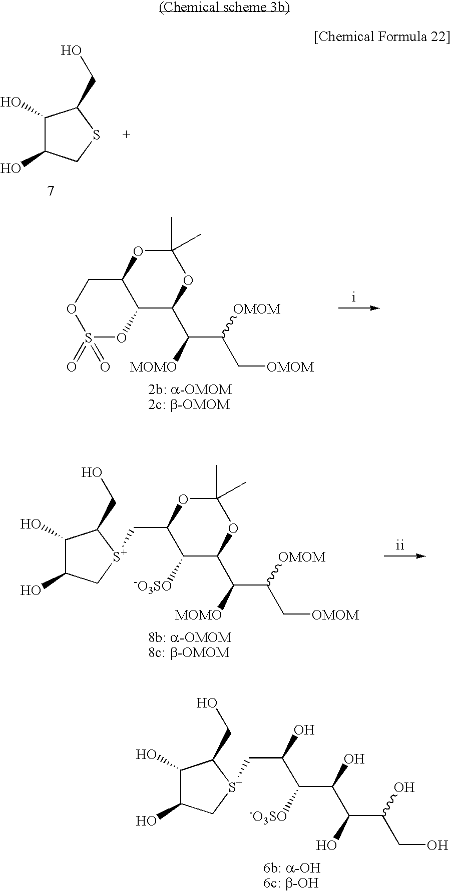

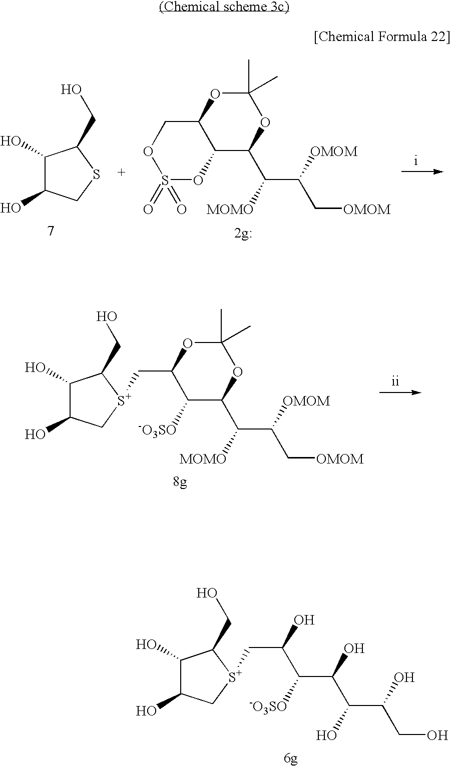

In the chemical scheme (3a), (3b) or (3c), for example, the compound (7) represents the thiosugar having as a skeleton a cyclic structure composed of four carbon atoms and one sulfur atom, including 1,4-dideoxy-1,4-epithio-D-arabinitol, and the compound (2) encompassing, e.g., the above compounds (2a), (2b), (2c), (2d) and (2g), represents the heptitol cyclosulfate with the protected hydroxy group. These compounds may be coupled to give the hydroxy group-protected cyclic sulfonium salts (8) encompassing the hydroxy group-protected cyclic sulfonium salts (8a), (8b), (8c), (8d) and (8g), respectively. The hydroxy group-protected cyclic sulfonium salts (8) are then subjected to deprotection of the protective hydroxy group therefrom leading to the kotalanol analogue (6) including the kotalanol analogues (6a), (6b), (6c), (6d) and (6g), respectively.

As illustrated by the chemical scheme (3a), (3b) or (3c), the compound (8) may be synthesized by the coupling reaction of the compound (2) with 1,4-dideoxy-1,4-epithio-D-arabinitol (7).

As a base to be used for the above coupling reaction, there may be used, for example, a carbonate such as potassium carbonate, sodium carbonate, lithium carbonate, magnesium carbonate, calcium carbonate, ammonium carbonate and so on. Potassium carbonate, sodium carbonate, lithium carbonate, etc. are preferred. The amount of the base to be used for the reaction may be in the range of approximately 10 to 50% with respect to the mole of the compound (2), although it may be lower than it.

A reaction solvent may include, for example, 1,1,1,3,3,3-hexafluoroisopropanol, 1,1,1,2,3,3,3-heptafluoroisopropanol, 2,2,3,3,3-pentafluoro-1-propanol, 1,1,2,2,3,3,3-heptafluoro-1-propanol and so on, and 1,1,1,3,3,3-hexafluoroisopropanol, 1,1,1,2,3,3,3-heptafluoroisopropanol, etc. are preferred. The reaction temperature may be in the range from room temperature to 100° C., preferably from 40 to 80° C. The reaction time may range from 24 to 72 hours.

The protective group of the compound (8) obtained by the above coupling step (C) can be deprotected therefrom by methods conventionally used for deprotection of a protective group thereby resulting in the formation of the compound (6).

As a regeant to be used for the deprotection of the protective group of the compound (8), there may be used, for example, trifluoroacetic acid aqueous solution, trichloroacetic acid aqueous solution, tribromoacetic acid aqueous solution, triiodoacetic acid aqueous solution, benzenesulfonic acid, p-toluenesulfonic acid, dilute sulfuric acid, dilute hydrochloric acid, and so on. Among these reagents, trifluoroacetic acid aqueous solution, trichloroacetic acid aqueous solution, tribromoacetic acid aqueous solution, and triiodoacetic acid aqueous solution are preferred, as well as trifluoroacetic acid aqueous solution and trichloroacetic acid aqueous solution are more preferred. When trifluoroacetic acid aqueous solution is used, the concentration of approximately 30% is preferred. The reaction temperature may be in the range of room temperature to 100° C., and the reaction time may be in the range of 30 minutes to four hours.

The hydroxy group-protected heptitol cyclosulfate (2) to be used for the present invention may be prepared, for example, by the chemical scheme (4) as described below. It is to be provided, however, that the below synthesis steps are described as being solely illustrative and they are not intended in any respect to limit the stereochemistry of the cyclic sulfate ester of the heptitol to be prepared by the present invention:

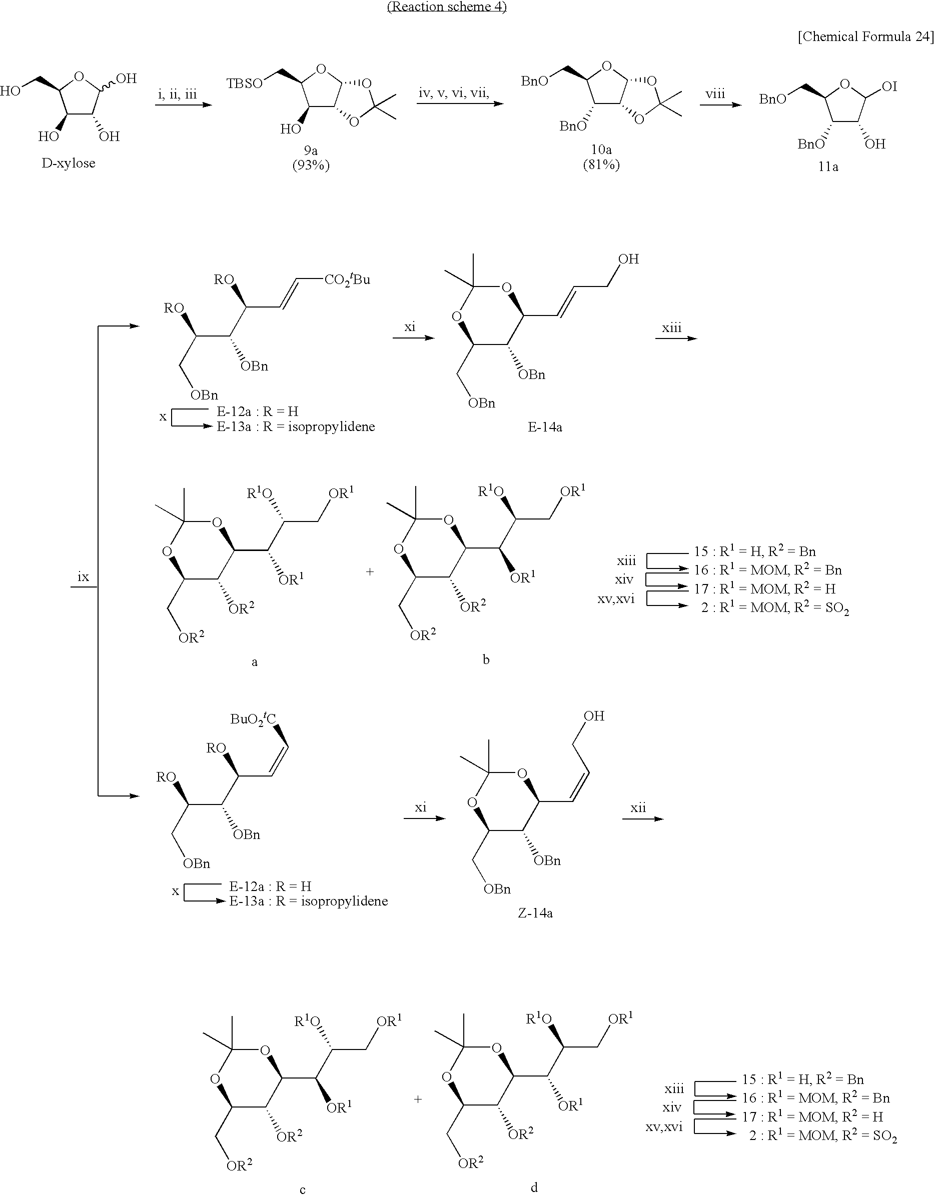

(wherein Bn is a benzyl group, TBS is tert-butyldimethylsilyl group, and MOM is methoxymethyl group).

The above synthesis method can efficiently produce the hydroxy group-protected heptitol cyclosulfate (2), for example, by using D-xylose as a starting material. As the hydroxy group-protected heptitol cyclosulfate (2) which can be prepared by this reaction (as shown in reaction scheme 4), there may be mentioned, for example, 2,4-O-isopropylidene-5,6,7-tri-O-methoxymethyl-D-glycero-L-allo-heptitol 1,3-cyclosulfate (2a), 4,6-O-isopropylidene-1,2,3-tri-O-methoxymethyl-D-glycero-D-gluco-heptitol 5,7-cyclosulfate (2b), 4,6-O-isopropylidene-1,2,3-tri-O-methoxymethyl-D-glycero-L-manno-heptitol 5,7-cyclosulfate (2c), and 4,6-O-isopropylidene-1,2,3-tri-O-methoxymethyl-D-glycero-D-allo-heptitol 5,7-cyclosulfate (2d).

The hydroxy group-protected heptitol cyclosulfate (2) can be prepared efficiently by the above reaction scheme (4).

The above hydroxy group-protected heptitol cyclosulfate (2) can be prepared by the above chemical scheme (4). More specifically, as illustrated therein, D-xylose, for example, used as a starting material, is reacted in the presence of acetone and an acid such as sulfuric acid, etc. (step i) and the resulting compound is reacted with an acid such as dilute hydrochloric acid, etc. (step ii), followed by reaction with a silane compound such as tert.-butyldimethylchlorosilane, etc., thereby yielding 5-O-tert-butyl-dimethylchlorosilyl-1,2-O-isopropylidene-α-D-xylofuranose (9a). As the chemical reactions, reagents, reaction conditions, operation conditions, etc. are used in conventional manner as are well known in the art, a description of details of them is omitted herefrom. This is applicable to the following description unless otherwise stated.

The remaining hydroxy group of the compound (9a) is then oxidized (step iv) and thereafter reduced (step v), followed by the deprotection of the tert.-butyldimethylchlorosilyl group (step vi) and thereafter the protection of two hydroxy group with benzyl group (step vii), thereby forming 3,5-di-O-benzyl-1,2-O-isopropylidene-α-D-ribofuranose (10a).

Thereafter, the isopropylidene group of the compound (10a) obtained above is deprotected (step viii) resulting in the formation of 3,5-di-O-benzyl-α- or β-D-ribofuranose (11a).

The above compound (11a) is then subjected to homologation (step ix) to form tert.-butyl (E)-5,7-di-O-benzyl-2,3-dideoxy-D-ribo-hepto-4-enoate (E-12a) and its Z-type isomer (Z-12a).

Next, the hydroxy group of the above compound (E-12a) is protected with isopropylidene group (step x) to form tert.-butyl (E)-5,7-di-O-benzyl-2,3-dideoxy-4,6-O-isopropylidene-D-ribo-hepto-2-enoate (E-13a) whose ester group is then reduced (step xi) to form (E)-5,7-di-O-benzyl-2,3-dideoxy-4,6-O-isopropylidene-D-ribo-hepto-2-enitol (E-14a).

The double bond of the above compound (E-14a) is then oxidized (step xii) to form 1,3-di-O-benzyl-2,4-O-isopropylidene-D-glycero-L-allo-heptitol (15a) and 5,7-di-O-benzyl-4,6-O-isopropylidene-D-glycero-D-gluco-heptitol (15b), followed by the protection of three hydroxy groups of each of the compounds (step xiii) to form 1,3-di-O-benzyl-2,4-O-isopropylidene-5,6,7-tri-O-methoxymethyl-D-glycero-L-allo-heptitol (16a) and 5,7-di-O-benzyl-4,6-O-isopropylidene-1,2,3-tri-O-methoxymethyl-D-glycero-D-gluco-heptitol (16b), respectively.

Further, the benzyl group of each of the above compounds (16a) and (16b) is deprotected (step xiv) to form 2,4-O-isopylidene-5,6,7-tri-O-methoxymethyl-D-glycero-L-allo-heptitol (17a) and 4,6-O-isopylidene-1,2,3-tri-O-methoxymethyl-D-glycero-D-gluco-heptitol (17b), respectively, and the resulting compounds (17a) and (17b) are esterified with sulfuric acid (step xv) resulting in the formation of a cyclic sulfate ester of the heptitol (2) with the hydroxy group protected.

On the other hand, the above compound (Z-12a) can be converted to the hydroxy group-protected heptitol cyclosulfate (2) in substantially the same manner as above.

More specifically, the hydroxy group of the compound (Z-12a) is protected with isopropylidene group (step x) to form tert.-butyl (Z)-5,7-di-O-benzyl-2,3-dideoxy-4,6-O-isopropylidene-D-ribo-hepto-2-enoate (Z-13a) whose ester group in turn is reduced (step xi) thereby resulting in the formation of (Z)-5,7-di-O-benzyl-2,3-dideoxy-4,6-O-isopropylidene-D-ribo-hepto-2-enitol (Z-14a).

Then, the double bond of the above compound (Z-14a) is oxidized (step xii) to form 5,7-di-O-benzyl-4,6-O-isopropylidene-D-glycero-D-manno-heptitol (15c) and 5,7-di-O-benzyl-4,6-O-isopropylidene-D-glycero-D-allo-heptitol (15d), and the three hydroxy groups of each of the resulting compounds are then protected (step xiii) to form 5.7-di-O-benzyl-4,6-O-isopropylidene-1,2,3-tri-O-methoxymethyl-D-glycero-D-manno-heptitol (16c) and 5,7-di-O-benzyl-4,6-O-isopropylidene-1,2,3-tri-O-methoxymethyl-D-glycero-D-allo-heptitol (16d), respectively.

Thereafter, the benzyl group of each of the above compounds (16c) and (16d) is deprotected (step xiv) to form 4,6-O-isopylidene-1,2,3-tri-O-methoxymethyl-D-glycero-D-manno-heptitol (17c) and 4,6-O-isopylidene-1,2,3-tri-O-methoxymethyl-D-glycero-D-allo-heptitol (17d), respectively, and the resulting compounds (17c) and (17d) are esterified with sulfuric acid (step xv) thereby forming the respective heptitol cyclosulfates (2c and 2d).

The protective group for the hydroxy group of the hydroxy group-protected cyclic sulfonium salt (2) as obtained above is then deprotected in conventional manner (step xvi) to form the kotalanol analogue (6).

As illustrated in the reaction scheme (4) above, for example, D-xylose used as a starting material is reacted by steps (i) to (xvi), inclusive, to form the hydroxy group-protected heptitol cyclosulfate (2) including, for example, 2,4-O-isopropylidene-5,6,7-tri-O-methoxymethyl-D-glycero-L-allo-heptitol 1,3-cyclosulfate (2a), 4,6-O-iso-propylidene-1,2,3-tri-O-methoxymethyl-D-glycero-D-gluco-heptitol 5,7-cyclosulfate (2b), 4,6-O-isopropylidene-1,2,3-tri-O-methoxymethyl-D-glycero-D-manno-heptitol 5,7-cyclosulfate (2c), and 4,6-O-isopropylidene-1,2,3-tri-O-methoxymethyl-D-glycero-D-allo-heptitol 5,7-cyclosulfate (2d), respectively.

The step (i) is an acetal-formation step by using D-xylose and acetone. For an acid to be used in this step, there may be used, for example, concentrated sulfuric acid, p-toluene sulfonic acid and concentrated hydrochloric acid, and concentrated sulfuric acid and p-toluene sulfonic acid are preferred. As a reagent for elimination of water formed during this step, there may be used, for example, anhydrous copper(II)sulfate, anhydrous magnesium sulfate, anhydrous sodium sulfate, etc., and anhydrous copper(II)sulfate is preferred. The reaction temperature may be room temperature or in the range of 30 to 50° C., and room temperature is preferred. The reaction time may be in the range of 10 to 14 hours.

The step (ii) is a decomposition reaction of by-products resulted from the above step. As an acid, there may be preferably used, e.g., hydrochloric acid in the concentration of 0.01% to 1%. The reaction temperature may be in the range of room temperature to 30 to 50° C., and room temperature is preferred. The reaction time may be in the range of 1 to 2 hours.

The step (iii) is for silylation of the acetal compound obtained by the step (i) with a silylating agent such as, e.g., tert.-butyldimethylchloro-silane. As a base to be used, there may be mentioned, for example, imidazole, pyridine, triethylamine and N-methylpiperidine, and imidazole and pyridine are preferred. A solvent may include, for example, an amide-type solvent such as dimethylformamide (DMF), dimethylacetamide, etc., and an ether-type solvent such as tetrahydrofuran, 1,4-dioxane, etc., although the amide-type solvent such as dimethylformamide (DMF), dimethylacetamide, etc., is preferred. The reaction temperature may be in the range of 0 to 20° C., although the reaction can be conducted around −10 to 30° C. The reaction time may be in the range of 1 to 6 hours and preferably 1 to 2 hours.

The step (iv) is an oxidation step for 5-O-tert.-butyldimethylsilyl-1,2-O-isopropylidene-α-D-xylofuranose (9a) obtained by the step (iii). As an oxidizing agent for use in this oxidation step, there may be mentioned, for example, a mild oxidizing agent including oxazolyl chloride ((COCl)₂), dimethylsulfoxide (DMSO), pyridinium chlorocjlomate (PCC), Collins reagent (chromic acid and pyridine), etc., and oxazolyl chloride ((COCl)₂) and dimethylsulfoxide (DMSO) are preferred. As a solvent, there may be used a chlorinated organic solvent such as dichloromethane, chloroform, carbon tetrachloride, etc., and dichloromethane is preferably used. The reaction temperature may be in the range of approximately −60 to −20° C., although the reaction at the temperature close to −60° C. is preferred. The reaction time may be in the range of 1 to 6 hours and preferably approximately 1 to 2 hours. As a base to be used after the reaction, there may be mentioned, for example, triethylamine, trimethylamine, pyridine, imidazole, etc., although triethylamine and trimethylamine, etc. are preferred.

The step (v) is a sterically selective reduction of the compound (a ketone) prepared by the step (iv). As a reducing agent to be used herein, there may be mentioned, for example, sodium boron hydride, lithium boron hydride, potassium boron hydride, sodium boron cyanohydride, borane-THF complex, borane-dimethylsulfide complex, etc., although sodium boron hydride, lithium boron hydride, potassium boron hydride and sodium boron cyanohydride are preferred. A solvent may include, for example, an alcohol such as an alcohol aqueous solution including ethanol aqueous solution, methanol aqueous solution, etc., and an alcohol including ethanol, methanol, etc., although the alcohol aqueous solution such as ethanol aqueous solution and methanol aqueous solution is preferred. The reaction temperature may be in the range of approximately −30° C. to room temperature, preferably in the range of approximately −30 to −10° C. The reaction time may be in the range of 1 to 8 hours and preferably 2 to 3 hours.

The step (vi) is for the deprotection of tert.-butyldimethylsilyl group. As a reagent to be used for the deprotection of the protective group, there may be mentioned, for example, dilute hydrochloric acid such as 0.1% to 1% HCl, etc., hydrogen fluoride, a quarternary ammonium halide such as tetrabutyl ammonium fluoride, etc., a carboxylic acid such as acetic acid, etc., a Louis acid such as boron trifluoride (BF₃), etc., although dilute hydrochloric acid is preferred. A solvent may include, for example, an ethereal solvent such as tetrahydrofuran (THF), 1,4-dioxane, diethyl ether, dipropyl ether, etc., with tetrahydrofuran (THF), 1,4-dioxane and diethyl ether being preferably used. The reaction temperature may be in the range of room temperature to 50° C., and room temperature is preferred. The reaction time may be in the range of 30 minutes to 4 hours and preferably 30 minutes to 1 hour.

The step (vii) is for benzylating reaction for benzylating the hydroxy group (for protection with the benzyl group). As a benzylating agent, there may be used, for example, a benzyl halide such as benzyl fluoride, benzyl chloride, benzyl bromide and benzyl iodide, although benzyl chloride, benzyl bromide and benzyl iodide, etc. are preferred. As a base, there may be used, for example, an alkali metal hydride such as sodium hydride, lithium hydride, potassium hydride, etc., an alkali metal amide such as sodium amide, lithium amide, potassium amide, etc., and an alkyllithium such as methyllithium, ethyllithium, propyllithium, butyllithium, etc., although the alkali metal hydride such as sodium hydride, lithium hydride, potassium hydride, etc. is preferred. As a solvent, there may be used, for example, an amide solvent such as dimethylformamide (DMF), dimethyl acetamide, etc., and an ethereal solvent such as tetrahydrofuran, 1,4-dioxane, etc., with the amide solvent such as dimethylformamide (DMF), dimethyl acetamide, etc. being preferred. The reaction temperature may be in the range of approximately −10° C. to 30° C., and the temperature at approximately 0° C. is preferred. The reaction time may be in the range of 1 to 7 hours and preferably 1 to 5 hours.

The reaction step (viii) is for the synthesis of 3,5-di-O-benzyl-α- and β-D-ribo-furanose (11a) by deprotection of the isopropylidene group of the compound (10). For the deprotection reaction, there may be used, for example, 0.5% sulfuric acid (dilute sulfuric acid), dilute hydrochloric acid, p-toluenesulfonic acid, a quarternary ammonium halide such as tetrabutylammonium fluoride, etc., a carboxylic acid such as acetic acid, etc., a Louis acid such as boron trifluoride (BF₃), etc., although 0.5% sulfuric acid (dilute sulfuric acid), dilute hydrochloric acid and p-toluenesulfonic acid are preferred. As a solvent, there may be used, for example, an ethereal solvent such as 1,4-dioxane, tetrahydrofuran, diethyl ether, etc., with 1,4-dioxane and tetrahydrofuran being preferred. The reaction temperature may be in the range of 80° C. to nearby reflux temperature (101° C.), although reflux temperature is preferred. The reaction time may be in the range of 1 to 5 hours and preferably 2 to 4 hours.

The steps (ix) and (x) will be described hereinafter, which relate to the synthesis of tert.-butyl (E) and (Z)-5,7-di-O-benzyl-2,3-dideoxy-4,6-O-isopropylidene-D-ribo-hepto-2-enoate (E-13a and Z-13a, respectively) from the compound (11a).

The step (ix) is involved in a homolation reaction (Wittig reaction) for reacting the compound (11) with a phosphonium ylide. As the phosphonium ylide, there may be used, for example, Ph₃P═CHCO₂t-Bu, etc. As a solvent, there may be used, for example, a chloromethane such as dichloromethane, chloroform, carbon tetrachloride, etc., and a chloroethane such as dichloroethane, trichloroethane, tetrachloroethane, pentachloroethane, hexachloroethane, etc., although chloromethanes are preferred. The reaction time may be reflux temperature, and the reaction time may range from 0.5 to 3 hours.

The step (x) is for the protection of the hydroxy group of the compounds (E-13a and Z-13a) prepared by the step (ix) (protection with isopropylidene group). As a reagent for the protection of the hydroxy group (protection with the isopropylidene group), there may be used, for example, 2,2-dimethoxypropane, etc. As an acid, there may be used, for example, p-toluenesulfonic acid, concentrated sulfuric acid, concentrated hydrochloric acid, etc., although p-toluenesulfonic acid and concentrated sulfuric acid are preferred. A solvent may include, for example, acetone, etc. The reaction temperature may range from room temperature to 50° C., with room temperature being preferred. The reaction time may be in the range of 1 to 4 hours and preferably 1 to 2 hours.

The step (xi) is for the synthesis of (E) and (Z)-5,7-di-O-benzyl-2,3-dideoxy-4,6-O-isopropylidene-D-ribo-hepto-2-enitol (E-14a and Z-14a) from the compounds (E-13a and Z-13a), respectively.

The step (xi) is for the reduction reaction for reducing the ester group of each of the compounds (E-13a and Z-13a) to the respective alcohol derivatives. As a reducing agent, there may be used, for example, diisobutylaluminium hydride (DIBAL), tri-tert.-butoxyaluminium hydride, lithium aluminum hydride, with diisobutylaluminum hydride (DIBAL) and tri-tert.-butoxyaluminum hydride are preferred. As a solvent, there may be used, for example, an ethereal solvent such as tetrahydrofuran, 1,4-dioxane, diethyl ether, etc., although tetrahydrofuran and 1,4-dioxane are preferred. The reaction temperature may be in the range of −60 to 40° C., and the reaction time may be in the range of 1 to 9 hours and preferably 5 to 7 hours.

A description will be made below regarding the step (xii) for the synthesis of 1,3-di-O-benzyl-2,4-O-isopropylidene-D-glycero-L-allo-heptitol (15a), 5,7-di-O-benzyl-4,6-O-isopropylidene-D-glycero-D-gluco-heptitol (15b), 5,7-di-O-benzyl-4,6-O-isopropylidene-D-glycero-D-manno-heptitol (15c), and 5,7-di-O-benzyl-4,6-O-isopropylidene-D-glycero-D-allo-heptitol (15c) from the compounds (E-14a and Z-14a), respectively.

The step (xii) is for oxidation of the double bond of each of the compounds (E-14a and Z-14a). As an oxidizing agent, there may be used, for example, osmium tetraoxide, potassium permanganese, etc., and osmium tetraoxide is preferred. As a base, there may be used, for example, N-methylmorpholine, N-oxide (NMO) and sodium oxide, although N-methylmorpholine N-oxide (NMO) is preferred. A solvent may include, for example, acetone-water, dioxane-water, TMF-water, etc., although acetone-water and dioxane-water are preferred. The reaction temperature may be reflux or in the range of 30 to 55° C., and reflux temperature is preferred. The reaction time may be in the range of 1 to 5 hours and preferably 2 to 3 hours.

A description will be made hereinbelow regarding the steps (xiii) and (xiv) for the synthesis of 1,3-di-O-benzyl-2,4-O-isopropylidene-5,6,7-tri-O-methoxymethyl-D-glycero-L-allo-heptitol (16a), 5,7-di-O-benzyl-4,6-O-isopropylidene-1,2,3-tri-O-methoxy-methyl-D-glycero-D-gluco-heptitol (16b), 5,7-di-O-benzyl-4,6-O-isopropylidene-1,2,3-tri-O-methoxymethyl-D-glycero-D-manno-heptitol (16c), and 5,7-di-O-benzyl-4,6-O-isopropylidene-1,2,3-tri-O-methoxymethyl-D-glycero-D-allo-heptitol (16d) from the compounds (15a, 15b, 15c and 15d), respectively.

The step (xiii) is to protect the three hydroxy groups of each of the compounds (15a, 15b, 15c and 15d) with methoxymethyl chloride. As a base, there may be used, for example, diisopropylethylamine, diisopropylmethylamine, triethylamine, tripropylamine and pyridine, although diisopropylethylamine, diisopropylmethylamine and triethylamine, etc. are preferred. A solvent may include, for example, an amide-type solvent such as dimethylformamide (DMF), dimethyacetamide, etc., although the ethereal solvent such as tetrahydrofuran, 1,4-dioxane, etc., although the amide-type solvent such as dimethylformamide (DMF), dimethyacetamide, etc. is preferred. The reaction temperature may range from room temperature to approximately 70° C., preferably from approximately 50 to 70° C. The reaction time may be in the range of 0.1 to 3 hours and preferably approximately 1 hour.

The step (xiv) is involved in a process for the synthesis of 2,4-O-isopropylidene-5,6,7-tri-O-methoxymethyl-D-glycero-L-allo-heptitol (17a), 4,6-O-isopropylidene-1,2,3-tri-O-methoxymethyl-D-glycero-D-gluco-heptitol (17b), 4,6-O-isopropylidene-1,2,3-tri-O-methoxymethyl-D-glycero-D-manno-heptitol (17c) and 4,6-O-isopropylidene-1,2,3-tri-O-methoxymethyl-D-glycero-D-allo-heptitol (17d) from the compounds (16a, 16b, 16c and 16d), respectively.

Specifically, the step (xiv) is for the deprotection reaction of the benzyl group of each of the compounds (16a, 16b, 16c and 16d). The deprotection reaction may be carried out by using H₂/Pd—C (palladium carbon), sodium hydrogen carbonate, Na/NH₃ and tetramethysilyl iodide, etc., although H₂/Pd—C (palladium carbon) and sodium hydrogen carbonate are preferred. A solvent may include, for example, an ethereal solvent such as 1,4-dioxane, dimethoxyethane, tetrahydrofuran, etc., although 1,4-dioxane and dimethoxyethane are preferred. The reaction temperature may be in the range of room temperature to approximately 70° C., preferably from approximately 50 to 60° C.

Then, a description will be made hereinafter regarding the step (xv) for the synthesis of 2,4-O-isopropylidene-5,6,7-tri-O-methoxymethyl-D-glycero-L-allo-heptitol 1,3-cyclosulfate (2a), 2,4-O-isopropylidene-5,6,7-tri-O-methoxymethyl-D-glycero-L-allo-heptitol 1,3-cyclosulfate (2a), 4,6-O-isopropylidene-1,2,3-tri-O-methoxymethyl-D-glycero-D-gluco-heptitol 5,7-cyclosulfate (2b), 4,6-O-isopropylidene-1,2,3-tri-O-methoxymethyl-D-glycero-D-manno-heptitol 5,7-cyclosulfate (2c) and 4,6-O-isopropylidene-1,2,3-tri-O-methoxymethyl-D-glycero-D-allo-heptitol 5,7-cyclosulfate (2d) from the compounds (17a, 17b, 17c and 17d), respectively.

The step (xv) relates to the cyclic sulfite esterification of the compounds (17a, 17b, 17c and 17d). As a reagent for the cyclic sulfite esterification, there may be used, for example, thionyl chloride, thionyl bromide, thionyl iodide, etc., and thionyl chloride and thionyl bromide are preferred. A base may include, for example, triethylamine, trimethylamine, pyridine, imidazole, etc., and triethylamine, trimethylamine and pyridine are preferred. As a solvent, there may be used, for example, a chloromethane such as dichloromethane, chloroform, carbon tetrachloride, etc., and a chloroethane such as dichloroethane, trichloroethane, tetrachlroethane, pentachloroethane, hexachloroethane, etc., although the chloromethane such as dichloromethane, chloroform, carbon tetrachloride, etc. is preferred. The reaction temperature may be in the range of from approximately −20° C. to 20° C., preferably from approximately −10 to 10° C. The reaction time may range from 20 minutes to 3 hours, preferably about 30 minutes.

The step (xvi) is involved in the oxidation of the cyclic sulfite ester of each of the compounds (17a, 17b, 17c and 17d) obtained by the step (xv) with sodium periodate or ruthenium chloride to give the objective cyclic sulfate esters (2a, 2b, 2c and 2d), respectively. For reaction conditions, there may be used, for example, sodium periodate, ruthenium n-hydrate, sodium hydrogen carbonate, etc. As a solvent, there may be used, for example, a mixed solvent such as carbon tetrachloride:acetonitrile:water (1:1:1). The reaction temperature may range from −10 to 40° C., preferably from −0° C. to room temperature.

Examples of the preferred conditions for each step of the above synthesis routes may be illustrated hereinafter:

(Step i) acetone, conc. Sulfuric acid, anhydrous copper(II)sulfate, room temperature, 12 hours;

(Step ii) 0.1% hydrochloric acid, room temperature, 1.5 hours;

(Step iii) TBSCl, imidazole, DMF, 0° C., 1 hour;

(Step iv) (COCl)₂, DMSO, CH₂Cl₂, −60° C., 1.5 hours followed by NEt₃;

(Step v) NaBH₄, EtOH, H₂, −20° C., 2.5 hours;

(Step vi) 0.2% aq. hydrochloric acid, THF, room temperature;

(Step vii) BnBLNaH, DMF, 0° C.;

(Step viii) 0.5% dilute sulfuric acid, dioxane, reflux temperature;

(Step ix) Ph₃P═CHCO₂^{t}Bu, CH₂Cl₂, reflux temperature;

(Step x) (CH₃)₂C(CH₃)₂, ρ-TsOH, acetone;

(Step xi) DIBAL, THF, −60° C. to room temperature;

(Step xii) OsO₄, NMO, acetone, H₂O, reflux temperature;

(Step xiii) MOMCl, ^{t}PrNEt, DMF, 60° C.;

(Step xiv) H₂, Pd—C, NaHCO₃, 1,4-dioxane, 60° C.;

(Step xv) SOCl₂, NEt₃, CH₂Cl₂, 0° C.; and

(Step xvi) NaIO₄, RuCl₃, n-H₂O, NaHCO₃, CH₃CN, CCl₄, H₂O, 0° C. to room temperature.

The hydroxy group-protected heptitol cyclosulfates (2e-1), (2e-2) as well as (2f-1) and (2f-2) to be used for the present invention, can be prepared, for example, in accordance with the chemical scheme (5) as described below:

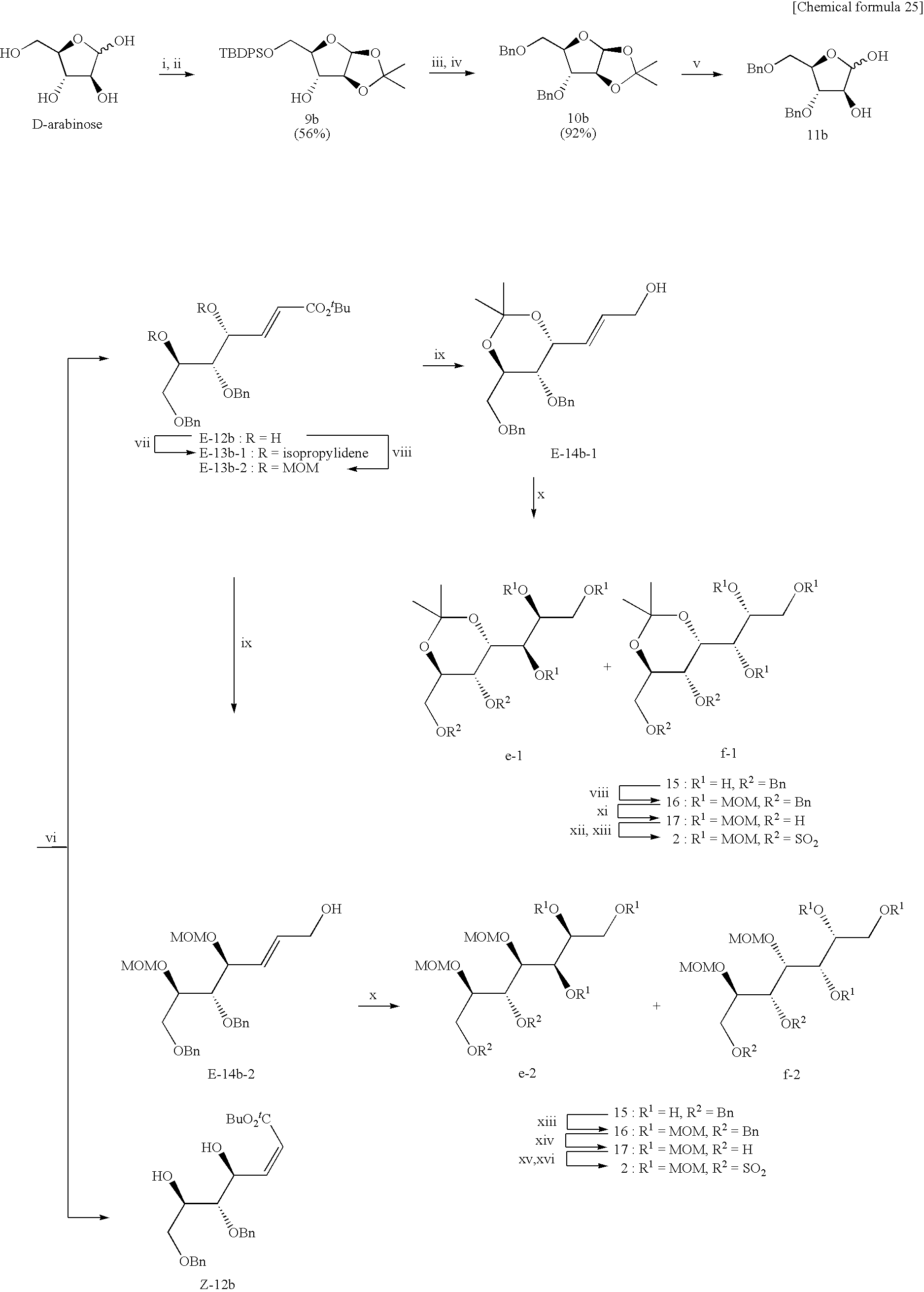

(wherein Bn is benzyl group, TBS is tert.-butyldimethylsilyl group, and MOM is methoxymethyl group).

These hydroxy group-protected heptitol cyclosulfates (2e-1), (2e-2), (2f-1) and (2f-2) can be prepared by using the reaction reagents, reaction conditions, etc. in substantially the same manner as in each step of the above reaction scheme (5).

Examples of the preferred conditions in each step of the above reaction scheme (5) are indicated as follows:

(Step i) TBDPSCl, imidazole, DMF, 0° C. to room temperature;

(Step ii) acetone, conc. sulfuric acid, anhydrous copper(II)sulfate, room temperature, 1 hour;

(Step iii) TBAF, THF-H₂O, 50° C., 3 hours;

(Step iv) BnBr, NaH, DMF, 0° C.;

(Step v) 1% dilute sulfuric acid, dioxane, reflux temperature;

(Step vi) Ph₃P═CHCO₂^{t}Bu, CH₂Cl₂, room temperature;

(Step vii) (CH₃)₂C(CH₃)₂, p-TsOH, acetone;

(Step viii) MOMCl, ⁱPr₂NEt, DMF, 60° C.;

(Step ix) DIBAL, THF, −60° C. to room temperature;

(Step x) OsO₄, NMO, acetone, H₂O, reflux temperature;

(Step xi) H₂, Pd—C, NaHCO₃, 1,4-dioxane, 60° C.;

(Step xii) SOCl₂, NEt₃, CH₂Cl₂, 0° C.; and

(Step xiii) NaIO₄, RuCl₃-n-H₂O, NaHCO₃, CH₃CN, CCl₄, H₂O, 0° C. to room temperature.

Among the hydroxy group-protected heptitol cyclosulfates (2) to be used for the present invention, the hydroxy group-protected heptitol cyclosulfates (2g) and (2h) can be prepared, for example, in accordance with the reaction scheme (6) as described below:

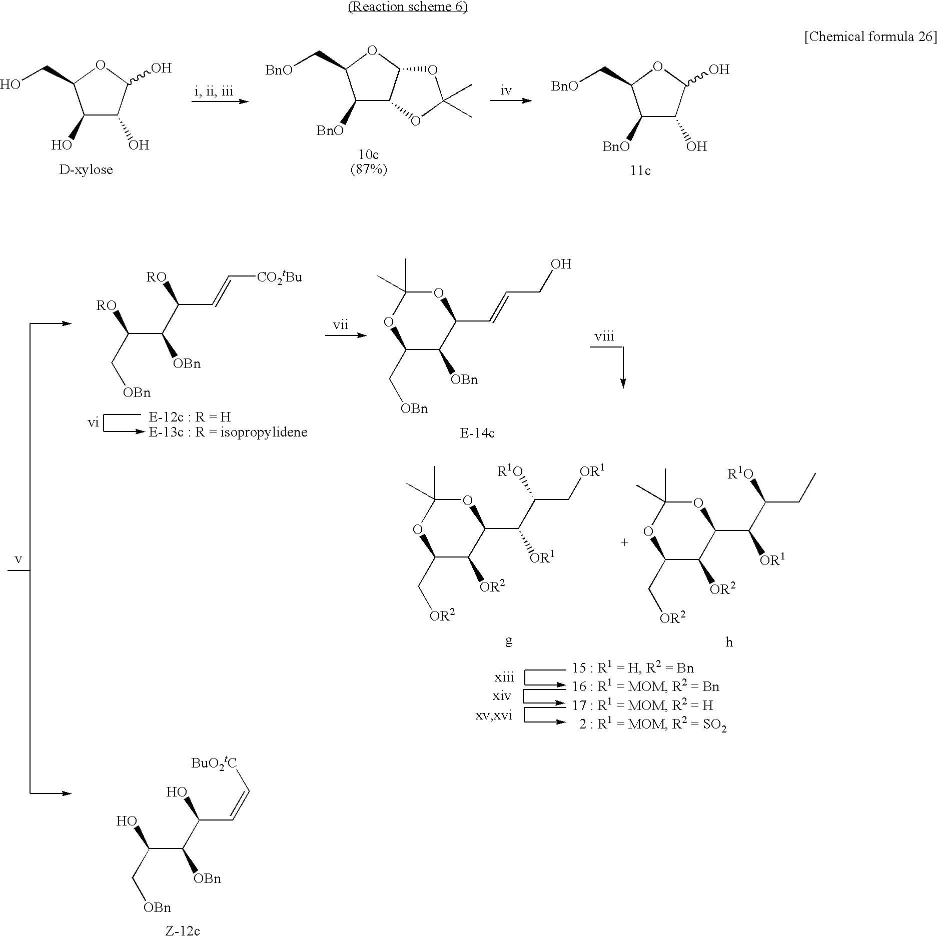

(wherein Bn is benzyl group, TBS is tert.-butyldimethylsilyl group, and MOM is methoxymethyl group).

These heptitol cyclosulfates (2g) and (2h) can be prepared by using the reaction reagents, reaction conditions, etc. in substantially the same manner as in each step of the above reaction scheme (6).

Examples of the preferred conditions in each step of the above reaction scheme (5) are indicated as follows:

(Step i) acetone, conc. sulfuric acid, anhydrous copper(II)sulfate, room temperature, 12 hours;

(Step ii) 0.1% hydrochloric acid, room temperature, 1.5 hours;

(Step iii) BnBr, NaH, DMF, 0° C.;

(Step iv) 1% dilute sulfuric acid, dioxane, reflux temperature;

(Step v) Ph₃P═CHCO₂^{t}Bu, CH₂Cl₂, room temperature;

(Step vi) (CH₃)₂C(CH₃)₂, p-TsOH, acetone;

(Step vii) DIBAL, THF, −60° C. to room temperature;

(Step viii) OsO₄, NMO, acetone, H₂O, reflux temperature;

(Step ix) MOMCl, ⁱPr₂NEt, DMF, 60° C.;

(Step x) H₂, Pd—C, NaHCO₃, 1,4-dioxane, 60° C.;

(Step xi) SOCl₂, NEt₃, CH₂Cl₂, 0° C.; and

(Step xii) NaIO₄, RuCl₃-n-H₂O, NaHCO₃, CH₃CN, CCl₄, H₂O, 0° C. to room temperature.

Furthermore, the present invention can provide a glycosidase inhibitor containing the above cyclic sulfonium salts (1) and/or (6) as well as an anti-diabetic agent or an anti-diabetic food containing the glycosidase inhibitor.

The cyclic sulfonium salts according to the present invention can be formulated singly or in combination with a pharmacologically acceptable carrier into preparations as a glycosidase inhibitor according to conventional preparation techniques. The glycosidase inhibitor according to the present invention can be applied particularly as an anti-diabetic agent. These preparations may be administered orally or parenterally to a mammalian animal such as humans, apes, and pets, e.g., dogs, cats, etc.

The amounts of the cyclic sulfonium salt in the preparations may be in the range of 1 to 90% by weight, preferably from 5 to 80% by weight, although they may vary with the kind of the cyclic sulfonium salts, preparations, etc.

The preparations of the present invention may include, for example, solid preparations including, e.g., tablets such as sublingual tablets, sugar-coated tablets, film coating tablets, two-layer tablets, multiplayer tablets, etc., capsules such as soft capsules, microcapsules, etc., granules, powders, troches, external preparations such as ointments, etc., suppositories, and liquid preparations including, e.g., oral preparations such as syrups, emulsions, suspensions, etc., injectable preparations such as subcutaneous, intravenous, intramuscular, intraperitoneal injections, intravenous drip injections, eye drops, inhalants, etc. The preparations may be administered safely through oral or parenteral routes.

As the pharmacologically acceptable carriers to be used for the preparations of the present invention, there may be used, for example, a variety of organic or inorganic carriers as used in the art conventionally as preparation materials. The carriers for use with solid preparations may include, for example, excipients, binders, lubricants, disintegrators, etc., and the carriers for use with liquid preparations may include, for example, solvents, solubility aids, suspending agents, isotonizing agents, buffers, painless agents, etc. As needed, there may be used, for example, additives such as antiseptics, antioxidants, coloring agents, sweeteners, etc.

As excipients to be used for the solid preparations of the present invention, there may be used, for example, glucose, lactose, sucrose, D-mannitol, D-sorbitol, starch, dextrin, hydroxypropylcellulose, sodium carboxymethylcellulose, gum arabic, pullulan, kaolin, microcrystalline cellulose, silicic acid, potassium phosphate, cacao butter, hydrogenated vegetable oils, etc. The binders may include, for example, sucrose, trehalose, dextrin, starch, gelatin, gum arabic, methylcellulose, carboxylmethylcellulose, sodium carboxylcellulose, microcrystalline cellulose, pullulan, hydroxypropylcellulose, hydroxypropylmethylcellulose, polyvinyl pyrrolidone, tragacanth powder, etc. The lubricants may include, for example, magnesium stearate, calcium stearate, talc, colloidal silica, etc. The disintegrators may include, for example, sodium carboxymethylcellulose, calcium carboxymethylcellulose, low-substituted hydroxypropylcellulose, dry starch, sodium alginate, agar powder, sodium hydrogen carbonate, calcium carbonate, etc.

As the solvents to be used for liquid preparations of the present invention, there may be used, for example, injectable water, physiolocal saline, Ringer's solution, alcohol, propylene glycol, polyethylene glycol, sesame oil, corn oil, olive oil, cottonseed oil, etc. The solubility aids may include, for example, polyethylene glycol, propylene glycol, D-mannitol, trehalose, benzylbenzoate, ethanol, Tris-aminomethane, cholesterol, triethanolamine, sodium carbonate, sodium citrate, sodium salicylate, sodium acetate, etc. The suspending agents may include, for example, surfactants such as stearyl triethanolamine, sodium lauryl sulfate, lauryl aminopropionate, lecithin, benzalkonium chloride, benzethonium chloride, glycerin monostearate, etc.; hydrophilic polymers such as polyvinyl alcohol, polyvinyl pyrrolidone, sodium carboxymethylcellulose, methyl cellulose, hydroxymethylcellulose, hydroxyethylcellulose, hydroxypropylcellulose, etc., polysorbates, polyoxyethylene hardened castor oil, etc. The isotonizing agents may include, for example, sodium chloride, glycerin, D-mannitol, D-sorbitol, glucose, etc. The buffers may include, for example, phosphates, acetates, carbonates, citrates, etc. The painless agents may include, for example, benzylalcohol, etc.

The antiseptics for use with the formulation of cyclic sulfonium salt preparations of the present invention to be added as needed may include, for example, p-oxybenzoates, chlorobutanol, benzyl alcohol, phenethyl alcohol, dehydroacetic acid, sorbic acid, etc. The anti-oxidants may include, for example, sulfites, ascorbates, etc. The coloring agents may include, for example, eible pigments such as edible red pigments, edible yellow pigments, edible blue pigments, etc., and natural pigments such as β-carotin, chlorophyll, Indian red, yellow iron(III)oxide, etc. The sweetners may include, for example, saccharin sodium, glycyrrhizinate dipotassium, aspartylphenylalanine, Stevia rebaudiana, etc.

The compounds and the medicines according to the present invention may be administered in various doses depending upon subjects, administration routes, diseases, symptoms, etc. When they are administered orally to adult patients with diabetes mellitus, for example, the compound of the present invention as an effective ingredient may be administered at a dose of normally approximately 0.01 to 100 mg per kg of body weight, preferably 0.05 to 30 mg per kg of body weight, more preferably 0.1 to 10 mg per kg of body weight. The dose may be preferably administered once to three times per day.

The cyclic sulfonium salt preparations of the present invention may be applied, for example, as an anti-diabetic agent for prevention or treatment of diabetes and diabetic complications, hyperlipidemia, arteriosclerosis, and so on. Diabetes may include, for example, type 1 diabetes mellitus, type 2 diabetes mellitus, gestational diabetes, etc. As the diabetic complications, there may be mentioned, for example, neurologic disorders, nephrosis, retinopathy, cataract, bone loss, diabetic hyperosmolar coma, infections such as respiratory infections, urinary tract infections, infections of digestive organs, infections of dermal tissues, infections of the lower limbs, etc., angiopathy such as diabetic gangrene, diabetic macroangiopathy, cerebrovascular infarction, peripheral angiopathy, etc. The hyperlipidemia may include, for example, hypertriglyceridemia, hypercholesterolemia, hypolipoproteinemia, postprandial hyperlipidemia, etc.

The cyclic sulfonium salt preparations according to the present invention may be used in combination with a combined medicine such as an antidiabetic agent, an agent for treating diabetic complications, an anti-hyperlipidemic agent, an antihypertensive agent, a diuretic, an antithrombic agent, a chemotherapeutic agent, an immunotherapeutic agent, an anti-obesity agent or the like.

The amount of such a combined medice may be selected in an appropriate manner on the basis of the dose to be applied clinically as a standard. The ratio of formulation of the combined medicine may be selected appropriately depending upon the subject, administration route, disease, symptoms, and so on. When the subject is a human, for example, the combined medicine may be applied in a concentration ranging from 0.01 to 100 parts by weight with respect to the weight of the cyclic sulfonium salt as an active ingredient of the preparation of the present invention.

As the antidiabetic agent to be used as the combined medicine, there may be used, for example, an animal-derived insulin preparation extracted from the kidneys of cattles or swines, a genetical recombinant insulin preparation such as genetical recombinant human insulin preparation, etc., insulin zinc, insulin zinc protamine, pioglitazone, rosigiitazone and the salt, e.g., hydrochloride, maleinate, etc., an agent for improving insulin resistance such as reglixane, netoglitazone, etc., α-glycosidase inhibitor such as voglibose, acarbose, miglitol, emiglitate, etc., a biguanide such as phenformin, metformin, buformin or a salt thereof, e.g., hydrochloride, fumarate, tartrate, etc.

The agent for treating diabetic complications may include, for example, an aldose reductase inhibitor such as tolrestat, epalrestat, zenarestat, zopolrestat, minalrestat, fidarestat, etc., a brain-derived neurotropic factor or an agent for increasing same, such as NGF, NT-3, BDNF, etc., an AGE inhibitor such as pimagedine, pyratoxatin, N-phenacylthiazolium, etc., and a cerebral vasodilatator such as tiapride, mexiletine, etc.

The antilipemic agent may include, for example, a statin compound as a cholesterol biosynthesis inhibitor, such as cerivastatin, pravastatin, simvastatin, lovastatin, atorvastatin, fluvastatin, itavastatin, rosuvastatin, pitavastatin or a sodium salt thereof; a fibrate-type compound such as bezafibrate, clofibrate, simfibrate, clinofibrate, etc.; an ACA inhibitor such as avasimibe, eflucimibe, etc., probucol- or nicotinic acid-type agent such as nicomol, niceritrol, etc.

The antihypertensive agent may include, for example, an angiotensin converting hypotensive agent such as captopril, enalapril, delapril, etc., an angiotensin II antagonist such as candesartan, cilexetil, losartan, eprosartan, valsartan, telmisartan, irbesartan, tasosartan, etc., a calcium antagonist such as manidipine, nifedipine, amlodipine, efonidipine, nicardipine, etc., a potassium channel opener such as levcromakalim, etc., clonidine, and so on.

The diuretic may include, for example, a xanthatine derivative such as sodium salicylate and theobromine, calcium salicylate and theobromine, etc., a thiazide preparation such as ethiazide, cyclopenthiazide, trichlormethiazide, hydrochlorothiazide, etc., an anti-aldosterone preparation such as spironolatone, triamterene, etc., a carbonic anhydrase inhibitor such as acetazolamide, etc., and a chlorobenzene sulfonamide-type preparation such as chlorthalidone, etc.

The antithrombic agent may include, for example, a heparin such as heparin sodium, heparin calcium, etc., a warfarin such as warfarin potassium, etc., an anti-thrombic agent such as angatroban, etc., a thrombolytic agent such as urokinase, tisokinase, etc., and a platelet aggregation inhibitor such as ticlopidine hydrochloride, etc.

As the chemotherapeutic agent, there may be used, for example, an antimetabolite such as cyclophosphamide, ifosfamide, etc., an alkylating agent such as methotrexate, 5-fluorouracil, etc., an anti-cancer antibiotic such as mitomycin, adriamycin, etc., a plant-derived anticancer agent such as vincristine, vindesine, taxol, etc., a platinum preparation such as cisplatin, carboplatin, etc., etopoxide, and so on. The immunotherapeutic agent may include, for example, an immunomodulator such as lentinan, sizofiran, krestin, etc., a microorganism or bacteria component such as a muramyl dipeptide derivative, picibanil, etc., a cytokine such as an interferon, e.g., interferon, IL-1, IL-2, IL-12, etc., and a colony-stimulating factor such as a granulocyte colony-stimulating factor, erythropoietin, etc.

The anti-obesity agent may include, for example, a central anti-obesity agent such as dexfenfluramine, fenfluramine, phentermine, sibutramine, amfepramone, dexamfetamine, mazindol, etc., a peptide-type appetite suppresser such as leptin, CNTF (ciliary neurotrophic factor), etc., and a cholecystokinin antagonist such as lintitript, etc.

As the medicine to be used in combination therewith, there may preferably be used, for example, an insulin preparation, an insulin resistance improving agent, an α-glycosidase inhibitor, and so on.

The anti-diabetic food according to the present invention may be prepared by mixing the glycosidase inhibitor of the present invention with various components of food. A form of the food is not limited to any particular one, and it may assume any form including, for example, a solid food, a semi-fluid food such as creams or jams, a gel-like food, beverages, and so on.

When the anti-diabetic food of the present invention is taken in the form of beverages, it may contain, in addition to the cyclic sulfonium salt as the active component, any additive such as saccharide, electrolyte, nutrients, vitamins, essence, colorant, flavor, artificial sweetener as so on, as needed. The saccharide may include, for example, glucose, sucrose, etc. The electrolyte may include, for example, sodium ion, potassium ion, chlorine ion, magnesium ion, phosphorus, organic acid, etc. Among these ions, sodium ion may be supplied from sodium chloride, sodium sulfate, sodium lactate, etc.; potassium ion being from potassium chloride, potassium phosphate, etc.; and magnesium ion being from magnesium chloride, magnesium sulfate, etc. Phosphorus may be supplied, for example, from an alkali metal or alkali earth metal phosphate such as sodium phosphate, potassium phosphate, etc. The organic acid may include, for example, lactic acid, sodium lactate, citric acid, sodium citrate, amino acids, alginic acid, gluconic acid, and so on.

The vitamins may include, for example, water-soluble or lipid-soluble vitamins, retinol palmitate, tocopherol, thiamine, riboflavin, sodium ascorbate, cholecalciferol, nicotinamide, calcium pantothenate, folic acid, biotin, and so on. As the colorants, flavoring materials and artificial sweeteners, there may be used, for example, any one that is used conventionally as food materials. These additives may be used singly or in combination with two or more.

When the food of the present invention is taken in a form of jelly, there may be added thereto, in addition to the above components, agar, gelatin, carrageenan, Jerangam, xanthan gum, pectin, sodium alginate, potassium alginate, and one or more of other viscosity-increasing polysaccharides to be used in conventional manner. The ratio of formulation of a gelling agent may be in an amount lower than approximately 2 parts by weight with respect to 100 parts by weight of a jelly.

The method for preparing the food of the present invention is not limited to a particular one, and a total amount of the food containing the cyclic sulfonium salt may be mixed simultaneously or each of the components of the food may be mixed separately.

### Examples

The cyclic sulfonium salt according to the present invention will be described in more detail, but it is to be understood that the present invention is not interpreted whatsoever as being limited to the examples as will be described below and that the following examples are described solely for the purpose of illustrating the present invention in more specific manner.

### Example 1

A mixture of 3,5-di-O-benzyl-1,2-O-isopropylidene-α-D-ribofuranose (10a) (22.9 g, 61.9 mmol), synthesized in the yield of 75% through seven steps from D-xylose, 1,4-dixane (170 ml) and 0.5% sulfuric acid (510 ml) was heated under reflux for 3 hours to give 3,5-di-O-benzyl-α- and -β-D-ribofuranose (11a) (20.5 g). This compound (19.8 g) was then heated under reflux for 1 hour together with tert.-butoxyethylene phenylphosphorane (29.6 g, 78.8 mmol) in dichloromethane (60 ml) to give tert.-butyl(E)-5,7-di-O-benzyl-2,3-dideoxy-D-ribo-hepto-2-enoate (E-12a) (18.7 g; yield, 73%) and tert.-butyl(Z)-5,7-di-O-benzyl-2,3-dideoxy-D-ribo-hepto-2-enoate (Z-12a) (3.8 g: yield, 15%).

The resulting compounds (E-12a) and (Z-12a) were each measured for melting point, specific rotatory power and infrared absorption spectrum. Their results are indicated as below:

TABLE 1
E-12: Colorless needles. Mp. 58-59° C. [α]_{D}²⁴ −5.74 (c = 1.30,
CHCl₃). IR (CHCl₃): 3460, 1705, 1655, 1454, 1393, 1369, 1315, 1226,
1215, 1157, 1088 cm⁻¹.
Z-12: Colorless needles. Mp. 61-62° C. [α]_{D}²⁴ +2.73 (c = 1.20,
CHCl₃). IR (CHCl₃): 3430, 1697, 1651, 1454, 1416, 1369, 1227, 1207,
1157, 1092 cm⁻¹.

The results of measurement for ¹H-NMR of the resulting compounds (E-12a) and (Z-12a) are indicated as below:

TABLE 2
E-12a: ¹H NMR (CDCl₃) (chemical shift): 1.48 (9H, s, (CH₃)₃C), 2.69 (1H, br d, J = 5.5 Hz,
OH), 3.15 (1H, br d, J = 4.0 Hz, OH), 3.53 (1H, dd, J = 7.2, 5.4, Hz, H-5), 3.60 (1H,
dd, J = 9.8, 5.5 Hz, H-7a), 3.67 (1H, dd, J = 9.8, 3.7 Hz, H-7b), 3.91 (1H, dddd, J = 7.2,
5.5, 5.5, 3.7 Hz, H-6), 4.51 (2H, br d, J = ca. 11.8 Hz, PhCH₂), 4.55 (1H, dddd, J = 5.4,
5.2, 4.0, 1.7 Hz, H-4), 4.56 (1H, d, J = 11.8 Hz, PhCH₂), 4.62 (1H, d, J = 11.2 Hz, PhCH₂),
6.06 (1H, dd, J = 15.8, 1.7 Hz, H-2), 6.99 (1H, dd, J = 15.8, 5.2, Hz, H-3), 7.23-7.38 (10H,
m, arom.).
Z-12a: ¹H NMR (CDCl₃) (chemical shift): 1.48 (9H, s, (CH₃)₃C), 3.23 (1H, d, J = 5.0 Hz,
OH), 3.62 (1H, dd, J = 9.8, 5.5 Hz, H-7a), 3.69 (1H, dd, J = 9.8, 3.0 Hz, H-7b), 3.71 (1H,
d, J = 5.1 Hz, OH), 3.77 (1H, dd, J = 7.7, 4.0 Hz, H-5), 3.85 (1H, dddd, J = 7.7, 5.5, 5.0,
3.0 Hz, H-6), 4.52/4.57 (each 1H, d, J = 11.8 Hz, PhCH₂), 4.64/4.76 (each 1H, d, J = 11.3 Hz,
PhCH₂), 5.26 (1H, dddd, J = 7.2, 5.1, 4.0, 1.5 Hz, H-4), 5.83 (1H, dd, J = 11.9, 1.5 Hz,
H-2), 6.34 (1H, dd, J = 11.9, 7.2 Hz, H-3), 7.25-7.36 (10H, m, arom.).

The results of measurement for ¹³C-NMR of the resulting compounds (E-12a) and (Z-12a) are indicated as below:

TABLE 3
E-12a: ¹³C NMR (CDCl₃) (chemical shift): 28.1 [(CH₃)₃C],
70.6 (C-7), 71.7 (C-6), 72.3 (C-4), 73.5/74.1 (PhCH₂) 80.4 [(CH₃)₃C],
81.4 (C-5), 123.6 (C-2), 127.9/127.99/128.04/128.1/128.5 (d, arom.),
137.46/137.52 (s, arom.), 145.1 (C-3), 165.6 (C-1).
Z-12a: ¹³C NMR (CDCl₃) (chemical shift): 28.1 [(CH₃)₃C], 69.0 (C-4),
70.7 (C-6), 70.9 (C-7), 73.4/73.7 (PhCH₂), 81.5 [(CH₃)₃C], 81.7 (C-5),
122.9 (C-2), 147.4 (C-3), 127.7/127.76/127.84/128.1/128.4 (d, arom.),
138.0/138.2 (s, arom.), 166.7 (C-1).

The results of measurement for mass analysis FAB (Fast Atom Bombardment)-MS and HR-FAB-MS of the resulting compounds (E-12a) and (Z-12a) are indicated as below:

TABLE 4

E-12: FABMS m/z: 429 [M + H]⁺ (pos.), FABHRMS m/z: 429.2293
(C₂₅H₃₃O₆ requires 429.2277).
Z-12: FABMS m/z: 429 [M + H]⁺ (pos.), FABHRMS m/z: 429.2302
(C₂₅H₃₃O₆ requires 429.2277).

### Example 2

A mixture of the compound (E-12a) (12 g, 28 mmol) obtained in Example 1, 2,2-dimethoxypropane (34.3 ml, 280 mmol), p-toluenesulfonic acid (24 mg) and acetone (120 ml) was stirred at room temperature for 1.5 hours to yield a colorless solid in the amount of 14.3 g. A small amount of this solid was recrystallized from n-hexane to yield (E)-5,7-di-O-benzyl-2,3-dideoxy-4,6-O-isopropylidene-D-ribo-hepto-2-enoate (E-13a) as a sample for analysis.

On the other hand, the compound (Z-12a) (1.7 g, 4.0 mmol) was treated in substantially the same manner as above to give a colorless solid in the amount of 1.87 g. A small amount of this solid was recrystallized from n-hexane to yield (Z)-5,7-di-O-benzyl-2,3-dideoxy-4,6-O-isopropylidene-D-ribo-hepto-2-enoate (Z-13a) as a sample for analysis.

The results of measurement for melting points, specific rotatory power and infrared absorption spectrum of the resulting compounds (E-13a) and (Z-13a) are indicated as below:

TABLE 5

E-13a: Colorless needles. Mp. 73-74° C. [α]_{D}²⁴ −29.2 (c = 1.01,
CHCl₃). IR (CHCl₃): 1709, 1654, 1454, 1369, 1312, 1211,
1153, 1096 cm⁻¹.
Z-13a: Colorless needles. Mp. 58-59° C. [α]_{D}²⁴ +96.6 (c = 4.60,
CHCl₃). IR (CHCl₃): 1717, 1651, 1454, 1369, 1207,
1157, 1096 cm⁻¹.

The results of measurement for ¹H-NMR of the resulting compounds (E-13a) and (Z-13a) are indicated as below:

TABLE 6
E-13a: ¹H NMR (CDCl₃) (chemical shift): 1.48/1.494 (each 3H, s, (CH₃)₂C), 1.491 (9H, s,
(CH₃)₃C), 3.30 (1H, dd, J = 9.7, 9.7, H-5), 3.63 (1H, dd, J = 10.9, 1.9 Hz, H-7a), 3.72 (1H,
dd, J = 10.9, 4.3 Hz, H-7b), 3.91 (1H, ddd, J = 9.7, 4.3, 2.0 Hz, H-6), 4.34 (1H, ddd, J = 9.7,
5.1, 1.5 Hz, H-4), 4.39/4.49 (each 1H, d, J = 10.6 Hz, PhCH₂), 4.55/4.66 (each 1H, d,
J = 12.2 Hz, PhCH₂), 6.09 (1H, dd, J = 15.6, 1.5 Hz, H-2), 6.97 (1H, dd, J = 15.6, 5.1,
H-3), 7.15-7.37 (10H, m, arom.).
Z-13a: ¹H NMR (CDCl₃) (chemical shift): 1.43 (9H, s, (CH₃)₃C), 1.46/1.57 (each 3H, s,
(CH₃)₂C), 3.32 (1H, br dd, J = 9.8, 9.5 Hz, H-5), 3.62 (1H, dd, J = 10.9, 2.0 Hz, H-7a),
3.71 (1H, dd, J = 10.9, 4.3 Hz, H-7b), 3.96 (1H, ddd, J = 9.8, 4.3, 2.0 Hz, H-6),
4.35/4.47 (each 1H, d, J = 10.6 Hz, PhCH₂), 4.55/4.65 (each 1H, d, J = 12.3 Hz, PhCH₂),
5.61 (1H, dd, J = 9.5, 8.9 Hz, H-4), 5.86 (1H, d, J = 11.5 Hz, H-2), 5.98 (1H, dd, J = 11.5,
8.9 Hz, H-3), 7.11-7.38 (10H, m, arom.).

The resulting compounds (E-13a) and (Z-13a) were each measured for ¹³C-NMR, and the results are indicated as below:

TABLE 7
E-13a: ¹³C NMR (CDCl₃) (chemical shift): 19.2/29.2 [(CH₃)₂C],
28.1 [(CH₃)₃C], 69.2 (C-7), 72.3 (C-4), 73.2 (C-6), 73.5/74.8 (PhCH₂),
74.5 (C-5), 80.4 [(CH₃)₃C], 98.9 [(CH₃)₂C], 124.2 (C-2), 127.7/127.9/
128.1/128.2/128.4/128.5 (d arom.), 137.2/138.1 (s arom.),
143.1 (C-3), 165.5 (C-1).
Z-13a: ¹³C NMR (CDCl₃) (chemical shift): 19.6/29.4 [(CH₃)₂C],
28.1 [(CH₃)₃C], 68.2 (C-4), 69.4 (C-7), 72.8 (C-6), 73.5/74.2 (PhCH₂),
73.9 (C-5), 80.8 [(CH₃)₃C], 98.8 [(CH₃)₂C], 125.6 (C-2), 127.6/127.7/
127.9/128.2/128.3 (d arom.), 137.7/138.2 (s arom.), 142.7 (C-3), 164.7
(C-1).

The resulting compounds (E-13a) and (Z-13a) were each measured for mass analysis FAB (Fast Atom Bombardment)-MS and HR-FAB-MS, and the results are indicated as below:

TABLE 8

E-13: FABMS m/z: 469 [M + H]⁺ (pos.), FABHRMS m/z:
469.2571 (C₂₈H₃₇O₆ requires 469.2590).
Z-13: FABMS m/z: 469 [M + H]⁺ (pos.), FABHRMS m/z:
469.2617 (C₂₈H₃₇O₆ requires 469.2590).

### Example 3

To a mixture of the crude compound (E-13a) (14.2 g), obtained by Example 2, and tetrahydrofuran (190 ml) was added a 1M toluene solution (64 ml, 64 mmol) of diisobutylaluminum hydride (DIBAL) at −78° C., and the resulting mixture was stirred at room temperature for 6 hours to yield (E)-5,7-di-O-benzyl-2,3-dideoxy-4,6-O-isopropylidene-D-ribo-hepto-2-enitol (E-14a) (10.3 g; yield, 93% from Z-12a).

The crude compound (Z-13a) (1.8 g) was treated in substantially the same manner to yield a colorless solid in the amount of 1.53 g. A small amount of the resulting solid material was recrystallized from a mixture of n-hexane and ethyl acetate to yield (Z)-5,7-di-O-benzyl-2,3-dideoxy-4,6-O-isopropylidene-D-ribo-hepto-2-enitol (Z-14a) as a sample for analysis.

The results of measurement for melting points, specific rotatory power and infrared absorption spectrum of the resulting compounds (E-14a) and (Z-14a) are indicated as below:

TABLE 9

E-14a: Colorless needles. Mp. 92-93° C. [α]_{D}²⁴ +9.1
(c = 1.22, CHCl₃). IR (nujol): 3479, 1651, 1203, 1169, 1111, 1096,
1054, 1029 cm⁻¹.
Z-14a: Colorless needles. Mp. 75-76° C. [α]_{D}²⁴ +76.5
(c = 2.37, CHCl₃). IR (CHCl₃): 3472, 1650, 1219, 1165,
1096, 1030 cm⁻¹.

The results of measurement for ¹H-NMR of the resulting compounds (E-14a) and (Z-14a) are indicated as below:

TABLE 10
E-14a: ¹H NMR (CDCl₃) (chemical shift): 1.35 (1H, br t-like, J = ca. 4.3 Hz, OH),
1.47/1.51 (each 3H, s, (CH₃)₂C), 3.30 (1H, dd, J = 8.0, 8.0 Hz, H-5), 3.65 (1H, dd, J = 9.1,
1.7 Hz, H-7a), 3.72 (1H, dd, J = 9.1, 3.6 Hz, H-7b), 3.89 (1H, ddd, J = 8.0, 3.6, 1.7 Hz,
H-6), 4.12 (2H, br t-like, J = ca. 4.3 Hz, H-1a and H-1b), 4.21, (1H, br dd, J = 8.0, 5.8 Hz,
H-4), 4.40/4.48 (each 1H, d, J = 10.8 Hz, PhCH₂), 4.56/4.67 (each 1H, d, J = 12.3 Hz,
PhCH₂), 5.72 (1H, ddt, J = 12.9, 5.8, 1.3 Hz, H-3), 6.00 (1H, dtd, J = 12.9, 4.3, 0.7 Hz,
H-2), 7.14-7.39 (10H, m, arom.).
Z-14a: ¹H NMR (CDCl₃) (chemical shift): 1.47/1.53 (each 3H, s, (CH₃)₂C), 1.99 (1H, br
t-like, J = ca.6.4 Hz, OH), 3.34 (1H, dd, J = 9.7, 9.7z, H-5), 3.64 (1H, dd, J = 11.0, 2.0 Hz,
H-7a), 3.73 (1H, dd, J = 11.0, 4.3 Hz, H-7b), 3.91 (1H, ddd, J = 9.7, 4.3, 2.0 Hz, H-6),
4.15 (1H, dddd, J = 13.0, 6.4, 6.4, 1.4 Hz, H-1a), 4.18 (1H, dddd, J = 13.0, 6.4, 6.4, 1.4 Hz,
H-1b), 4.44/4.50 (each 1H, d, J = 10.7 Hz, PhCH₂), 4.57/4.67 (each 1H, d, J = 12.2 Hz,
PhCH₂), 4.63 (1H, ddd, J = 9.7, 8.3, 0.9 Hz, H-4), 5.57 (1H, ddt, J = 11.2, 8.3, 1.4 Hz,
H-3), 5.89 (1H, dtd, J = 11.2, 6.4, 0.9 Hz, H-2), 7.13-7.38 (10H, m, arom).

The results of measurement for ¹³C-NMR of the resulting compounds (E-14a) and (Z-14a) are indicated as below:

TABLE 11
E-14a: ¹³C NMR (CDCl₃) (chemical shift): 19.4/29.4 [(CH₃)₂C],
62.9 (C-1), 69.3 (C-7), 73.0 (C-6), 73.5/74.4 (PhCH₂), 73.7 (C-4),
74.4 (C-5), 98.7 [(CH₃)₂C], 127.6/127.9/128.0/128.2/
128.3/128.4 (d. arom.), 128.8 (C-3), 133.3 (C-2), 137.7/138.2 (s, arom.).
Z-14a: ¹³C NMR (CDCl₃) (chemical shift): 19.4/29.4 [(CH₃)₂C],
59.2 (C-1), 69.3 (C-7), 69.4 (C-4), 73.1 (C-6), 73.5/74.7 (PhCH₂),
74.3 (C-5), 98.8 [(CH₃)₂C],
127.7/127.9/128.1/128.2/128.4/128.5 (d arom.), 129.9 (C-3), 133.5 (C-2),
137.2/138.1 (s arom.).

The results of measurement for mass analysis FAB (Fast Atom Bombardment)-MS and HR-FAB-MS of the resulting compounds (E-14a) and (Z-14a) are indicated as below:

TABLE 12
E-14: FABMS m/z: 399 [M + H]⁺ (pos.), FABHRMS m/z: 399.2180
(C₂₄H₃₁O₅ requires 399.2171).
Z-14: FABMS m/z: 399 [M + H]⁺ (pos.), FABHRMS m/z: 399.2184
(C₂₄H₃₁O₅ requires 399.2171).

### Example 4

A mixture of the compound (E-14a) (6.2 g, 15.6 mmol), obtained by Example 3, 0.045 M osmium tetraoxide aqueous solution (17.2 ml, 0.78 mmol), N-methylmorpholine N-oxide (3.65 g, 31.2 mmol), acetone (55 ml) and water (5 ml) was heated under reflux for 2.5 hours to yield an oily material in the amount of 6.9 g. A small amount of the resulting mixture was separated by column chromatography yielding 1,3-di-O-benzyl-2,4-O-isopropylidene-D-glycero-L-allo-heptitol (15a) and 5,7-di-O-benzyl-4,6-O-isopropylidene-D-glycero-D-gluco-heptitol (15b) as samples for analysis.

The crude compound (Z-14a) (1.5 g) was treated in substantially the same manner as above to give an oily material in the amount of 1.68 g. A small amount of the resulting mixture was separated by column chromatography to yield 5,7-di-O-benzyl-4,6-O-isopropylidene-D-glycero-D-manno-heptitol (15c) and 5,7-di-O-benzyl-4,6-O-isopropylidene-D-glycero-D-allo-heptitol (15d) as samples for analysis.

The results of measurement for specific rotatory power and infrared absorption spectrum of the compounds (15a), (15b), (15c) and (15d) are indicated as below:

TABLE 13
15a: Colorless oil. [α]_{D}²⁴ −2.1 (c = 1.13, CHCl₃). IR (neat): 3418,
1454, 1384, 1265, 1203, 1169, 1107, 1030 cm⁻¹.
15b: Colorless plates. Mp. 121-122° C. [α]_{D}²⁴ +11.7 (c = 1.09, CHCl₃).
IR (CHCl₃): 3526, 1451, 1384, 1215, 1204, 1165, 1092, 1042 cm⁻¹.
15c: Colorless prisms. Mp. 91-92° C. [α]_{D}²⁴ +8.5 (c = 2.46, CHCl₃).
IR (CHCl₃): 3479, 1520, 1423, 1223, 1092, 1045 cm⁻¹.
15d: Colorless needles. Mp. 82-83° C. [α]_{D}²⁴ +4.2 (c = 1.38, CHCl₃).
IR (CHCl₃): 3533, 1520, 1454, 1223, 1204, 1092 cm⁻¹.

The results of measurement for ¹H-NMR of the resulting compounds (15a), (15b), (15c) and (15d) are indicated as below:

TABLE 14
15a: ¹H NMR (CDCl₃) (chemical shift): 1.45/1.49 (each, 3H, s, (CH₃)₂C), 2.25 (1H, dd,
J = 7.4, 4.8 Hz, OH), 3.11 (1H, dd, J = 3.6 Hz, OH), 3.19 (1H, d, J = 6.5 Hz, OH),
3.53 (1H, ddd, J = 11.7, 7.4, 4.5 Hz, H-7a), 3.59 (1H, dd, J = 9.8, 9.2 Hz, H-3), 3.61 (1H, ddd,
J = 11.7, 4.8, 4.5 Hz, H-7b), 3.66 (1H, dd, J = 11.2, 2.1 Hz, H-1a), 3.75 (1H, ddd, J = 6.5,
3.8, 1.6 Hz, H-5), 3.79 (1H, dd, J = 11.2, 3.6 Hz, H-1b), 3.86 (1H, ddd, J = 4.5, 4.5,
1.6 Hz, H-6), 3.88 (1H, ddd, J = 9.2, 3.6, 2.1 Hz, H-2), 3.99 (1H, dd, J = 9.8, 3.8 Hz,
H-4), 4.45/4.52 (each 1H, d, J = 11.0 Hz, PhCH₂), 4.58/4.71 (each 1H, d, J = 12.0 Hz,
PhCH₂), 7.16-7.39 (10H, m, arom.).
15b: ¹H NMR (CDCl₃) (chemical shift): 1.46/1.50 (each, 3H, s, (CH₃)₂C), 2.24 (1H, t, J = 6.0 Hz,
OH), 2.67 (1H, dd, J = 9.5 Hz, OH), 3.13 (1H, d, J = 1.2 Hz, OH), 3.63 (1H, dd,
J = 10.9, 2.0 Hz, H-7a). 3.68 (1H, dd, J = 10.9, 4.6 Hz, H-7b), 3.70 (2H, dd-like, J = ca.
6.0, 5.4 Hz, H-1a and H-1b), 3.76 (1H, dd, J = 9.7, 9.7 Hz, H-5), 3.80-3.88 [3H, m, H-2,
H-3, including one-proton double multiplets due to H-4 at [symbol] 3.83 (J = 9.7 Hz)],
3.90 (1H, ddd, J = 9.7, 4.6, 2.0 Hz, H-6), 4.49/4.62 (each 1H, d, J = 10.9 Hz, PhCH₂),
4.56/4.64 (each 1H, d, J = 12.1 Hz, PhCH₂), 7.17-7.39 (10H, m, arom.).
15c: ¹H NMR (CDCl₃) (chemical shift): 1.45/1.51 (each 3H, s, (CH₃)₂C), 2.04 (1H, br s,
OH), 2.37 (1H, br s, OH), 2.48 (1H, br s, OH), 3.64 (1H, dd, J = 11.0, 2.2 Hz, H-7a),
3.70 (1H, dd, J = 11.0, 4.5 Hz, H-7b), 3.71 (1H, dd, J = 9.8, 9.8 Hz, H-5), 3.72-3.76 (3H,
m, H-3, H-2, H-1a), 3.79 (1H, dm, J = ca. 10.5 Hz, H-1b), 3.92 (1H, ddd, J = 9.8, 4.5,
2.2 Hz Hz, H-6), 4.01 (1H, d, J = 9.8 Hz, H-4), 4.49/4.59 (each 1H, d, J = 11.0 Hz,
PhCH₂), 4.56/4.64 (each 1H, d, J = 12.2 Hz, PhCH₂), 7.17-7.38 (10H, m, arom).
15d: ¹H NMR (CDCl₃) (chemical shift): 1.45/1.50 (each 3H, s, (CH₃)₂C), 2.31 (1H, br s,
OH), 2.79 (1H, br d, J = 2.2 Hz, OH), 2.94 (1H, br d, J = 3.7 Hz, OH), 3.68 (1H, dd, J = 11.2,
2.2 Hz, H-7a), 3.71 (1H, br dm, J = ca. 10.5 Hz, H-1a), 3.73-3.78 (1H, m, H-2),
3.78-3.84 [4H, m, H-1b, H-3, including two one-proton doublet of doublets due to H-7b
and H-5 at [symbol] 3.79 (J = 11.2, 3.7 Hz) and [symbol] 3.81 (J = 9.6, 9.6 Hz), respectively],
3.89 (1H, ddd, J = 9.6, 3.7, 2.2 Hz, H-6), 3.93 (1H, dd, J = 9.6, 4.4 Hz, H-4), 4.56/4.59 (each
1H, d, J = 10.8 Hz, PhCH₂), 4.57/4.70 (each 1H, d, J = 12.2 Hz, PhCH₂), 7.16-7.39 (10H,
m, arom).

The results of measurement for ¹³C-NMR of the resulting compounds (15a), (15b), (15c) and (15d) are indicated as below:

TABLE 15
15a: ¹³C NMR (CDCl₃) (chemical shift): 19.0/29.3 [(CH₃)₂C], 64.7 (C-7), 69.1 (C-1),
69.9 (C-6), 71.3 (C-5), 71.7 (C-3), 73.3 (C-2), 73.7/73.9 (PhCH₂), 75.6 (C-4),
99.2 [(CH₃)₂C], 127.8/128.1/128.2/128.3/128.4/128.6 (d, arom.), 137.0/137.8 (s, arom).
15b: ¹³C NMR (CDCl₃) (chemical shift): 19.4/29.3 [(CH₃)₂C], 64.0 (C-1), 68.6 (C-2),
69.3 (C-7), 69.6 (C-5), 73.1 (C-6), 73.4 (C-3), 73.5/74.6 (PhCH₂), 75.7 (C-4),
99.0 [(CH₃)₂C], 127.7/127.97/128.03/128.4/128.5 (d, arom.), 137.6/138.0 (s, arom.).
15c: ¹³C NMR (CDCl₃) (chemical shift): 19.5/29.2 [(CH₃)₂C], 64.2 (C-1), 69.27 (C-2),
69.33 (C-7), 69.7 (C-5), 72.1 (C-4), 72.2 (C-3), 73.2 (C-6), 73.5/74.6 (PhCH₂),
98.9 [(CH₃)₂C], 127.7/127.96/128.00/128.4/128.5 (d arom.), 137.6/138.0 (s arom.).
15d: ¹³C NMR (CDCl₃) (chemical shift): 19.3/29.2 [(CH₃)₂C], 64.2 (C-1), 69.3 (C-7),
71.8 (C-2), 72.3 (C-5), 73.3 (C-3 and C-4), 73.7/74.2 (PhCH₂), 73.9 (C-6),
98.9 [(CH₃)₂C], 127.8/128.06/128.13/128.3/128.4/128.7 (d arom.), 136.8/137.8 (s arom.).

The results of measurement for mass analysis FAB (Fast Atom Bombardment)-MS and HR-FAB-MS) of the resulting compounds (15a), (15b), (15c) and (15d) are indicated as below:

TABLE 16
15a: FABMS m/z: 433 [M + H]⁺ (pos.), FABHRMS m/z: 433.2213
(C₂₄H₃₃O₇ requires 433.2226).
15b: FABMS m/z: 433 [M + H]⁺ (pos.), FABHRMS m/z: 433.2239
(C₂₄H₃₃O₇ requires 433.2226).
15c: FABMS m/z: 433 [M + H]⁺ (pos.), FABHRMS m/z: 433.2239
(C₂₄H₃₃O₇ requires 433.2226).
15d: FABMS m/z: 433 [M + H]⁺ (pos.), FABHRMS m/z: 433.2200
(C₂₄H₃₃O₇ requires 433.2226).

### Example 5

A mixture (6.9 g) of the compounds (15a) and (15b), obtained by Example 4, was reacted with methoxymethyl chloride (MOMCl, 14.6 ml, 192 mmol) in diisobutylethylamine (55.6 ml, 319 mmol) and dimethylformamide (200 ml) at 60° C. for 1 hour to yield 1,3-di-O-benzyl-2,4-O-isopropylidene-5,6,7-tri-O-methoxymethyl-D-glycero-L-allo-heptitol (16a) (6.0 g; yield, 68% from E-14a) and 5,7-di-O-benzyl-2,4-O-isopropylidene-5,6,7-tri-O-methoxymethyl-D-glycero-D-gluco-heptitol (16b) (2.0 g; yield, 23% from E-14a).

A mixture (925 mg) of the resulting compounds (15a) and (15b) was treated in accordance with the above processes to yield 5,7-di-O-benzyl-4,6-O-isopropylidene-1,2,3-tri-O-methoxymethyl-D-glycero-D-manno-heptitol (16c) (527 mg; yield, 45% from Z-12a and 5,7-di-O-benzyl-4,6-O-isopropylidene-1,2,3-tri-O-methoxymethyl-D-glycero-D-allo-heptitol (16d) (489 mg; yield, 42% from Z-12a).

The results of measurement for boiling points, specific rotatory powers and infrared absorption spectra of the compounds (16a), (16b), (16c) and (16d) are indicated respectively as below:

TABLE 17
16a: Colorless oil. Bp. 239-243° C./0.004 mHg. [α]_{D}²⁴ +37.9
(c = 1.90, CHCl₃). IR (neat): 1454, 1381, 1258, 1207, 1150, 1026 cm⁻¹.
16b: Colorless oil. Bp. 245-248° C./0.005 mHg. [α]_{D}²⁴ −3.0
(c = 1.53, CHCl₃). IR (neat): 1454, 1381, 1257, 1204, 1150, 1110,
1034 cm⁻¹.
16c: Colorless oil. [α]_{D}²⁴ +4.45 (c = 1.37, CHCl₃).
IR (neat): 1458, 1381, 1258, 1204, 1151, 1108, 1034 cm⁻¹.
16d: Colorless oil. [α]_{D}²⁴ +14.1 (c = 1.40, CHCl₃).
IR (neat): 1454, 1381, 1258, 1207, 1150, 1107, 1034 cm⁻¹.

The results of measurement for ¹H-NMR of the resulting compounds (16a), (16b), (16c) and (16d) are indicated as below:

TABLE 18
16a: ¹H NMR (CDCl₃) (chemical shift): 1.45/1.48 (each 3H, s, (CH₃)₂C), 3.32/3.39/
3.44 (each 3H, s, OCH₂OCH₃), 3.66 (1H, dd, J = 11.0, 2.2 Hz, H-1a), 3.720 (1H, dd, J = 9.5,
9.5 Hz, H-3), 3.722 (1H, dd, J = 10.8, 6.5 Hz, H-7a), 3.73 (1H, dd, J = 11.0, 4.5 Hz,
H-1b), 3.77 (1H, dd, J = 10.8, 4.0 Hz, H-7b), 3.88 (1H, ddd, J = 9.5, 4.5, 2.2 Hz, H-2),
3.98 (1H, ddd, J = 7.2, 6.5, 4.0 Hz, H-6), 4.01 (1H, dd, J = 9.5, 1.0 Hz, H-4), 4.07 (1H,
dd, J = 7.2, 1.0 Hz, H-5), 4.44/4.67 (each 1H, d, J = 10.8 Hz, PhCH₂), 4.57/4.66 (each
1H, d, J = 12.2 Hz, PhCH₂), 4.59/4.60 (each 1H, d, J = 6.4 Hz, OCH₂OCH₃),
4.73/4.828 (each 1H, d, J = 6.7 Hz, OCH₂OCH₃), 4.812/4.826 (each 1H, d, J = 7.0 Hz, OCH₂OCH₃),
7.17-7.38 (10H, m, arom.).
16b: ¹H NMR (CDCl₃) (chemical shift): 1.47/1.48 (each 3H, s, (CH₃)₂C),
3.33/3.34/3.41 (each 3H, s, OCH₂OCH₃), 3.64 (1H, dd, J = 11.2, 5.8 Hz, H-1a), 3.68 (1H,
dd, J = 10.9, 2.0 Hz, H-7a), 3.74 (1H, dd, J = 9.7, 9.7 Hz, H-5), 3.77 (1H, dd, J = 10.9,
4.3 Hz, H-7b), 3.81 (1H, dd, J = 11.2, 2.1 Hz, H-1b), 3.90 (1H, ddd, J = 9.7, 4.3, 2.0 Hz,
H-6), 3.96 (1H, dd, J = 9.7, 0.9 Hz, H-4), 4.02 (1H, ddd, J = 6.9, 5.8, 2.1 Hz, H-2),
4.08 (1H, dd, J = 6.9, 0.9 Hz, H-3), 4.49/4.71 (each 1H, d, J = 10.8 Hz, PhCH₂),
4.56/4.68 (each 1H, d, J = 12.2 Hz, PhCH₂), 4.62/4.64 (each 1H, d, J = 6.4 Hz, OCH₂OCH₃),
4.730/4.89 (each 1H, d, J = 6.6 Hz, OCH₂OCH₃), 4.732/4.77 (each 1H, d, J = 6.6 Hz,
OCH₂OCH₃), 7.17-7.38 (10H, m, arom.).
16c: ¹H NMR (CDCl₃) (chemical shift): 1.48 (6H, s, (CH₃)₂C), 3.36/3.39/3.40 (each 3H,
s, OCH₂OCH₃), 3.68 (1H, dd, J = 11.0, 2.0 Hz, H-7a), 3.73 (1H, dd, J = 11.2, 4.5 Hz,
H-1a), 3.76 (1H, dd, J = 11.0, 4.3 Hz, H-7b), 3.77 (1H, dd, J = 9.7, 9.7 Hz, H-5),
3.86 (1H, ddd, J = 6.9, 4.5, 2.5 Hz, H-2), 3.91 (1H, ddd, J = 9.7, 4.3, 2.0 Hz, H-6), 3.92 (1H,
dd, J = 11.2, 2.4 Hz, H-1b), 3.97 (1H, dd, J = 9.7, 1.2 Hz, H-4), 4.11 (1H, dd, J = 6.9,
1.2 Hz, H-3), 4.50/4.76 (each 1H, d, J = 11.2 Hz, PhCH₂), 4.56/4.67 (each 1H, d, J = 12.2,
PhCH₂), 4.65/4.66 (each 1H, d, J = 6.4 Hz, OCH₂OCH₃), 4.71/4.72 (each 1H, d, J = 6.7 Hz,
OCH₂OCH₃), 4.74/4.91 (each 1H, d, J = 6.7 Hz, OCH₂OCH₃), 7.21-7.38 (10H,
m, arom).
16d: ¹H NMR (CDCl₃) (chemical shift): 1.46/1.50 (each 3H, s, (CH₃)₂C),
3.352/3.354/3.41 (each 3H, s, OCH₂OCH₃), 3.63 (1H, dd, J = 10.9, 2.0 Hz, H-7a),
3.70 (1H, dd, J = 10.9, 4.6 Hz, H-7b), 3.72 (1H, dd, J = 10.9, 4.9 Hz, H-1a), 3.75 (1H, dd, J = 9.8,
9.8 Hz, H-5), 3.87 (1H, ddd, J = 9.8, 4.6, 2.0, Hz, H-6), 3.94 (1H, dd, J = 10.9, 2.3 Hz,
H-1b), 4.01 (1H, ddd, J = 7.2, 4.9, 2.3 Hz, H-2), 4.10 (1H, dd, J = 7.2, 1.2 Hz, H-3),
4.12 (1H, dd, J = 9.8, 1.2 Hz, H-4), 4.46/4.68 (each 1H, d, J = 10.9 Hz, PhCH₂),
4.55/4.64 (each 1H, d, J = 12.2 Hz, PhCH₂), 4.64/4.66 (each 1H, d, J = 6.3 Hz,
OCH₂OCH₃), 4.72/4.755 (each 1H, d, J = 6.6 Hz, OCH₂OCH₃), 4.764/4.79 (each 1H, d, J = 6.3 Hz,
OCH₂OCH₃), 7.20-7.38 (10H, m, arom).

The results of measurement for ¹³C-NMR of the resulting compounds (16a), (16b), (16c) and (16d) are indicated as below:

TABLE 19
16a: ¹³C NMR (CDCl₃) (chemical shift): 19.0/29.4 [(CH₃)₂C], 55.4/55.6/
56.0 (OCH₂OCH₃), 67.9 (C-7), 69.6 (C-1), 70.7 (C-3), 73.47 (C-2),
73.52/73.8 (PhCH₂), 74.0 (C-4), 76.6 (C-6), 77.3 (C-5), 96.7/97.1/97.4
(OCH₂OCH₃), 98.7 [(CH₃)₂C],
127.6/127.7/127.9/128.3/128.4 (d, arom.), 137.8/138.2 (s, arom.).
16b: ¹³C NMR (CDCl₃) (chemical shift): 19.0/29.5 [(CH₃)₂C], 55.4/55.7/
56.0 (OCH₂OCH₃), 68.2 (C-1), 69.86 (C-5), 69.94 (C-7), 71.2 (C-4),
73.4/73.6 (PhCH₂), 73.7 (C-6), 76.6 (C-3), 78.3 (C-2), 96.7/97.4/98.3
(OCH₂OCH₃), 98.5 [(CH₃)₂C], 127.5/127.6/127.7/127.9/128.26/
128.29 (d, arom.), 138.2/138.3 (s, arom.).
16c: ¹³C NMR (CDCl₃) (chemical shift): 19.1/29.5 [(CH₃)₂C], 55.3/55.7/
55.9 (OCH₂OCH₃), 67.8 (C-1), 69.96 (C-7), 70.02 (C-5), 71.9 (C-4),
73.5/73.6 (PhCH₂), 73.6 (C-6), 76.6 (C-3), 77.3 (C-2), 96.8/97.1/98.1
(OCH₂OCH₃), 98.5 [(CH₃)₂C], 127.56/127.62/127.9/128.27/
128.30 (d arom.), 138.3/138.4 (s arom.).
16d: ¹³C NMR (CDCl₃) (chemical shift): 19.0/29.4 [(CH₃)₂C], 55.3/55.9/
56.0 (OCH₂OCH₃), 68.2 (C-1), 69.6 (C-7), 71.2 (C-5), 73.4/74.1
(PhCH₂), 73.6 (C-6), 74.5 (C-4), 76.1 (C-3), 76.7 (C-2), 96.8/97.0/97.1
(OCH₂OCH₃), 98.8 [(CH₃)₂C], 127.6/127.7/127.8/128.30/
128.34 (d arom.), 138.0/138.3 (s arom.).

The results of measurement for mass analysis FAB (Fast Atom

Bombardment)-MS and HR-FAB-MS of the resulting compounds (16a), (16b), (16c) and (16d) are indicated as below:

TABLE 20
16a: FABMS m/z: 565 [M + H]⁺ (pos.), FABHRMS m/z: 565.2983
(C₃₀H₄₅O₁₀ requires 565.3012).
16b: FABMS m/z: 565 [M + H]⁺ (pos.), FABHRMS m/z: 565.3013
(C₃₀H₄₅O₁₀ requires 565.3012).
16c: FABMS m/z: 565 [M + H]⁺ (pos.), FABHRMS m/z: 565.3002
(C₃₀H₄₅O₁₀ requires 565.3013).
16d: FABMS m/z: 565 [M + H]⁺ (pos.), FABHRMS m/z: 565.3026
(C₃₀H₄₅O₁₀ requires 565.3013).

### Example 6

The compound (16a) (2.85 g, 5.05 mmol), obtained by Example 5, was contact-reduced with palladium carbon in the presence of sodium hydrogen carbonate (400 mg) in 1,4-dioxane (45 ml) to yield 2,4-O-isopropylidene-5,6,7-tri-O-methoxymethyl-D-glycero-L-allo-heptitol (17a) (1.87 g; yield, 96%)

The above processes were followed by using the compounds (16b) (864 mg, 1.53 mmol), (16c) (287 mg, 0.51 mmol) and (16d) (275 mg, 0.49 mmol), respectively, to give 4,6-O-isopropylidene-1,2,3-tri-O-methoxymethyl-D-glycero-D-gluco-heptitol (17b) (567 mg; yield, 96%), 4,6-O-isopropylidene-1,2,3-tri-O-methoxymethyl-D-glycero-D-manno-heptitol (17c) (179 mg; yield, 92%) and 4,6-O-isopropylidene-1,2,3-tri-O-methoxymethyl-D-glycero-D-allo-heptitol (17d) (170 mg; yield, 91%).

The results of measurement for boiling points, specific rotatory powers and infrared absorption spectra of the resulting compounds (17a), (17b), (17c) and (17d) are indicated respectively as below:

TABLE 21
17a: Colorless oil. Bp. 176-179° C./0.004 mmHg. [α]_{D}²⁴ +37.6
(c = 2.89, CHCl₃). IR (neat): 3418, 1454, 1384, 1265, 1207, 1151, 1108,
1034 cm⁻¹.
17b: Colorless plates. Mp. 64.5-65° C. Bp. 173-175° C./0.005 mmHg.
[α]_{D}²⁴ −59.2 (c = 1.27, CHCl₃). IR (neat): 3422,
1454, 1384, 1261, 1204, 1152, 1109, 1034 cm⁻¹.
17c: Colorless oil. [α]_{D}²⁴ −9.1 (c = 3.16, CHCl₃).
IR (neat): 3420, 1458, 1384, 1261, 1204, 1153, 1108, 1030 cm⁻¹.
17d: Colorless oil. [α]_{D}²⁴ +22.4 (c = 3.30, CHCl₃).
IR (neat): 3421, 1458, 1385, 1261, 1207, 1151, 1108, 1034 cm⁻¹.

The results of measurement for ¹H-NMR of the resulting compounds (17a), (17b), (17c) and (17d) are indicated respectively as below:

TABLE 22
17a: ¹H NMR (CDCl₃) (chemical shift): 1.39/1.50 (each 3H, s, (CH₃)₂C), 2.17 (1H,
dd-like J = ca. 7.0, 6.0 Hz, OH), 3.38/3.41/3.44 (each 3H, s, OCH₂OCH₃), 3.66 (1H, ddd,
J = 8.6, 8.6, 2.3 Hz, H-3), 3.68 (1H, dd, J = 10.3, 7.2 Hz, H-7a), 3.72-3.79 [3H, m, H-1a
H-2, including one-proton doublet of doublets due to H-7b at [symbol] 3.76 (J = 10.3, 5.7 Hz)],
3.82 (1H, d, J = 2.3 Hz, OH), 3.83-3.88 (1H, m, H-1b), 3.89 (1H, dd, J = 8.6, 6.0 Hz,
H-4), 3.91 (1H, dd, J = 6.0, 2.6 Hz, H-5), 3.97 (1H, ddd, J = 7.2, 5.7, 2.6 Hz, H-6),
4.65 (2H, s-like, OCH₂OCH₃), 4.74/4.76 (each 1H, d, J = 6.7 Hz, OCH₂OCH₃), 4.78/4.85 (each
1H, d, J = 6.0 Hz, OCH₂OCH₃).
17b: ¹H NMR (CDCl₃) (chemical shift): 1.42/1.49 (each 3H, s, (CH₃)₂C), 2.14 (1H,
dd-like J = ca. 7.8, 4.6 Hz, OH), 3.41/3.42/3.46 (each 3H, s, OCH₂OCH₃), 3.63 (1H, dd,
J = 11.2, 3.2 Hz, H-1a), 3.72-3.82 [5H, m, H-5, H-6, H-7a, and OH, including
one-proton doublet of doublets due to H-1b at [symbol] 3.75 (J = 11.2, 2.3 Hz)],
3.82-3.86 (2H, m, H-4, H-7b), 3.97 (1H, ddd, J = 8.3, 3.2, 2.3 Hz, H-2), 4.06 (1H, dd, J = 8.3, 2.9 Hz,
H-3), 4.66/4.67 (each 1H, d, J = 6.6 Hz, OCH₂OCH₃), 4.76 (2H, br d, J = ca. 6.8 Hz
OCH₂OCH₃), 4.48 (1H, d, J = 6.9 Hz, OCH₂OCH₃), 4.87 (1H, d, J = 6.3 Hz OCH₂OCH₃).
17c: ¹H NMR (CDCl₃) (chemical shift): 1.43/1.48 (each 3H, s, (CH₃)₂C), 2.16 (1H, br
dd-like J = ca 7.2, 4.0 Hz, OH), 3.39/3.42/3.49 (each 3H, s, OCH₂OCH₃), 3.55 (1H, d, J = 4.8 Hz,
OH), 3.71 (1H, dd, J = 11.0, 3.1 Hz, H-1a), 3.72 (1H, ddd, J = 9.6, 9.6, 4.8 Hz,
H-5), 3.76-3.87 [4H, m, H-6, H-7a, H-7b, including one-proton doublet of doublet of
doublets due to H-2 at [symbol] 3.85 (J = 8.4, 3.1, 2.4 Hz)], 3.90 (1H, dd, J = 11.0, 2.4 Hz,
H-1b), 3.95 (1H, dd, J = 9.6, 2.4 Hz, H-4), 4.07 (1H, dd, J = 8.4, 2.4 Hz, H-3),
4.68/4.700 (1H, d, J = 6.5 Hz, OCH₂OCH₃), 4.69/4.74 (1H, d, J = 6.5 Hz, OCH₂OCH₃),
4.702/4.85 (1H, d, J = 6.5 Hz, OCH₂OCH₃).
17d: ¹H NMR (CDCl₃) (chemical shift): 1.39/1.49 (each 3H, s, C(CH₃)₂), 2.19 (1H, br
t-like, J = ca 5.3 Hz, OH), 3.37/3.42/3.43 (each 3H, s, OCH₂OCH₃), 3.648 (1H, ddd, J = 9.1,
9.1, 2.1 Hz, H-5), 3.650 (1H, dd, J = 10.5, 6.9 Hz, H-1a), 3.71-3.78 [3H, m, H-6,
H-7a including one-proton doublet of doublets due to H-1b at [symbol] 3.73 (J = 10.5, 4.7 Hz)],
3.85 (1H, ddd, J = 7.2, 5.3, 5.3 Hz, H-7b), 3.88 (1H, dd, J = 9.1, 4.4 Hz, H-4),
3.98 (1H, dd, J = 4.4, 3.1 Hz, H-3), 4.112 (1H, ddd, J = 6.9, 4.7, 3.1 Hz, H-2), 4.114 (1H, d,
J = 2.1, OH), 4.63/4.64 (each 1H, d, J = 6.7, OCH₂OCH₃), 4.77/4.80 (each 1H, d, J = 6.7,
OCH₂OCH₃), 4.78/4.83 (each 1H, d, J = 6.4 Hz, OCH₂OCH₃).

The results of measurement for ¹³C-NMR of the resulting compounds (17a), (17b), (17c) and (17d) are indicated respectively as below:

TABLE 23
17a: ¹³C NMR (CDCl₃) (chemical shift): 19.4/29.3 [(CH₃)₂C], 55.6/56.0/
56.4 (OCH₂OCH₃), 63.2 (C-1), 66.2 (C-3), 67.3 (C-7), 72.0 (C-4),
72.9 (C-2), 76.5 (C-6), 81.0 (C-5), 97.0/97.6/98.67 (OCH₂OCH₃), 98.70
[(CH₃)₂C].
17b: ¹³C NMR (CDCl₃) (chemical shift): 19.2/29.2 [(CH₃)₂C], 55.6/55.7/
56.2 (OCH₂OCH₃), 63.1 (C-7), 63.3 (C-5), 67.1 (C-1), 73.0 (C-4), 73.1
(C-6), 77.3 (C-3), 77.5 (C-2), 96.9/97.0/99.5 (OCH₂OCH₃), 99.2
[(CH₃)₂C].
17c: ¹³C NMR (CDCl₃) (chemical shift): 19.4/29.4 [(CH₃)₂C], 55.6/55.7/
56.8 (OCH₂OCH₃), 63.0 (C-7), 63.4 (C-5), 67.5 (C-1), 72.5 (C-4), 73.1
(C-6), 76.17 (C-3), 76.22 (C-2), 97.0/97.6/99.0 (OCH₂OCH₃), 99.1
[(CH₃)₂C].
17d: ¹³C NMR (CDCl₃) (chemical shift): 19.3/29.2 [(CH₃)₂C], 55.3/55.8/
56.1 (OCH₂OCH₃), 63.4 (C-7), 64.6 (C-5), 67.5 (C-1), 72.2 (C-4), 73.0
(C-6), 76.3 (C-2), 79.3 (C-3), 96.6/96.87/96.93 (OCH₂OCH₃), 98.6
[(CH₃)₂C].

The results of mass analysis FAB (Fast Atom Bombardment)-MS and HR-FAB-MS for the resulting compounds (17a), (17b), (17c) and (17d) are indicated respectively as below:

TABLE 24
17a: FABMS m/z: 385 [M + H]⁺ (pos.), FABHRMS m/z: 385.2072
(C₁₆H₃₃O₁₀ require 385.2074).
17b: FABMS m/z: 385 [M + H]⁺ (pos.), FABHRMS m/z: 385.2085
(C₁₆H₃₃O₁₀ require 385.2074).
17c: FABMS m/z: 385 [M + H]⁺ (pos.), FABHRMS m/z: 385.2097
(C₁₆H₃₃O₁₀ require 385.2074).
17d: FABMS m/z: 385 [M + H]⁺ (pos.), FABHRMS m/z: 385.2090
(C₁₆H₃₃O₁₀ require 385.2074).

### Example 7

The compound (17a) (1.0 g, 2.6 mmol), obtained by Example 6, was treated with thionyl chloride (250 μ1, 3.4 mmol) in triethylamine (0.9 ml, 6.5 mmol) and dichloromethane solution (20 ml) while stirring at 0° C. for 30 minutes, thereby yielding an oily material in the amount of 1.3 g. The resulting oily material was then oxidized with sodium periodate (1.67 g, 7.8 mmol) and ruthenium chloride n-hydrate (100 mg) in the presence of sodium hydrogen carbonate (800 mg, 9.5 mmol) in a mixed solution of carbon tetrachloride (20 ml), acetonitrile (20 ml) and water (20 ml) to yield 2,4-O-isopropylidene-5,6,7-tri-O-methoxymethyl-D-glycero-L-allo-heptitol 1,3-cyclosulfate (2a) (593 mg; yield, 51%).

The above processes were followed by using the compound (17b) (539 mg, 1.4 mmol), the compound (17c) (154 mg, 0.4 mmol) and the compound (17d) (148 mg, 0.39 mmol), respectively, to give 4,6-O-isopropylidene-1,2,3-tri-O-methoxymethyl-D-glycero-D-gluco-heptitol 5,7-cyclosulfate (2b) (356 mg; yield, 57%), 4,6-O-isopropylidene-1,2,3-tri-O-methoxymethyl-D-glycero-D-manno-heptitol 5,7-cyclosulfate (2c) (134 mg; yield, 78%) and 4,6-O-isopropylidene-1,2,3-tri-O-methoxymethyl-D-glycero-D-allo-heptitol 5,7-cyclosulfate (2d) (74 mg; yield, 47%), respectively.

The results of measurement for specific rotatory power and infrared absorption spectra of the resulting compounds (2a), (2b), (2c) and (2d) are indicated respectively as below:

TABLE 25

2a: Colorless oil. [α]_{D}²² +5.02 (c = 2.57, CHCl₃),
IR (neat): 1454, 1416, 1250, 1203,
1151, 1110, 1026 cm⁻¹.
2b: Colorless prisms. [α]_{D}²⁴ −29.4 (c = 2.50, CHCl₃).
Mp. 79-80° C. IR (CHCl₃): 1416,
1231, 1200, 1150, 1107, 1018 cm⁻¹.
2c: Colorless oil. [α]_{D}²⁴ −9.9 (c = 6.00, CHCl₃).
IR (neat): 1458, 1420, 1253, 1204, 1153,
1112, 1034 cm⁻¹.
2d: Colorless oil. [α]_{D}²⁴ −12.5 (c = 5.68, CHCl₃).
IR (neat): 1458, 1420, 1252, 1204,
1152, 1114, 1026 cm⁻¹.

The results of measurement for ¹H-NMR of the resulting compounds (2a), (2b), (2c) and (2d) are indicated respectively as below:

TABLE 26
2a: ¹H-NMR (CDCl₃) (chemical shift): 1.45/1.56 (each 3H, s, (CH₃)₂C),
3.38/3.41/3.43 (each 3H, s, OCH₂OCH₃), 3.74 (1H, dd, J = 11.0, 5.0 Hz,
H-7a), 3.77 (1H, dd, J = 11.0, 3.4 Hz, H-7b), 3.92-3.77 (2H, m, H-5 and
H-6), 4.22 (1H, ddd, J = 10.3, 9.8, 4.8 Hz, H-2), 4.25 (1H, dd, J = 9.8,
2.7 Hz, H-4), 4.46 (1H, dd, J = 10.3, 4.8 Hz, H-1eq), 4.60 (1H, dd, J =
10.3, 10.3, H-1ax), 4.64/4.66 (each 1H, d, J = 6.7 Hz, OCH₂OCH₃),
4.74/4.77 (each 1H, d, J = 6.7 Hz, OCH₂OCH₃), 4.75/4.78 (each 1H, d,
J = 6.7 Hz, OCH₂OCH₃), 4.87 (1H, dd, J = 9.8, 9.8 Hz, H-3).
2b: ¹H NMR (CDCl₃) (chemical shift): 1.46/1.56 (each 3H s, (CH₃)₂C),
3.38/3.40/3.46 (each 3H, s, OCH₂OCH₃), 3.60 (1H, dd, J = 11.5, 6.0 Hz,
H-1a), 3.81 (1H, dd, J = 11.5, 2.6 Hz, H-1b), 3.94 (1H, dd, J = 6.6,
1.7 Hz, H-3), 4.01 (1H, dd, J = 6.6, 6.0, 2.6 Hz, H-2), 4.25 (1H, ddd, J =
10.6, 9.5, 4.9 Hz, H-6), 4.26 (1H, dd, J = 9.5, 1.7 Hz, H-4), 4.47 (1H, dd,
J = 10.6, 4.9 Hz, H-7eq), 4.61 (1H, dd, J = 10.6, 10.6 Hz, H-7ax), 4.63/
4.65 (each 1H, d, J = 6.6 Hz, OCH₂OCH₃), 4.71 (1H, dd, J = 9.5,
9.5 Hz, H-5), 4.74 (2H, d, J = 6.6 Hz, OCH₂OCH₃), 4.78 (1H, d, J =
6.6 Hz, OCH₂OCH₃), 4.81 (1H, d, J = 6.6 Hz, OCH₂OCH₃).
2c: ¹H NMR (CDCl₃) (chemical shift): 1.47/1.55 (each 3H, s, (CH₃)₂C),
3.39/3.42/3.46 (each 3H, s, OCH₂OCH₃), 3.72 (1H, dd, J = 11.2, 3.6 Hz,
H-1a), 3.80 (1H, ddd, J = 7.8, 3.6, 2.4 Hz H-2), 3.91 (1H, dd, J = 11.2,
2.4 Hz, H-1b), 3.97 (1H, dd, J = 7.8, 1.6 Hz H-3), 4.26 (1H, ddd, J =
10.3, 9.8, 4.8 Hz, H-6), 4.29 (1H, dd, J = 9.8, 1.6 Hz, H-4), 4.49 (1H, dd,
J = 10.3, 4.8 Hz H-7eq), 4.62 (1H, dd, J = 10.3, 10.3 Hz, H-7ax), 4.68
(2H, s, OCH₂OCH₃), 4.71/4.73 (each 1H, d, J = 6.7, OCH₂OCH₃),
4.71 (1H, dd, J = 9.8, 9.8 Hz, H-3), 4.72/4.80 (1H, d, J = 6.2 Hz,
OCH₂OCH₃).
2d: ¹H NMR (CDCl₃) (chemical shift): 1.43/1.56 (each 3H, s, (CH₃)₂C),
3.38 (3H, s, OCH₂OCH₃), 3.43 (6H, s, OCH₂OCH₃), 3.69 (1H, dd, J =
10.5, 4.0 Hz, H-1a), 3.85 (1H, ddd, J = 7.7, 4.0, 2.8 Hz, H-2), 3.87 (1H,
ddd, J = 10.5, 2.8 Hz, H-1b), 3.97 (1H, dd, J = 7.7, 2.0 Hz, H-3), 4.24
(1H, ddd, J = 10.5, 9.8, 4.8 Hz, H-6), 4.43 (1H, dd, J = 9.8, 2.0 Hz,
H-4), 4.47 (1H, dd, J = 10.5, 4.8 Hz, H-7eq), 4.60 (1H, dd, J = 10.5,
10.5 Hz, H-7ax), 4.66 (2H, s, OCH₂OCH₃), 4.74/4.78 (each 1H, d, J =
6.5 Hz, OCH₂OCH₃), 4.77 (2H, s, OCH₂OCH₃), 4.83 (1H, dd, J = 9.8,
9.8 Hz, H-5).

The results of measurement for ¹³C-NMR of the resulting compounds (2a), (2b), (2c) and (2d) are indicated respectively as below:

TABLE 27
2a: ¹³C-NMR (CDCl₃) (chemical shift): 19.1/28.7 [(CH₃)₂C], 55.5/55.8/56.2 (OCH₂OCH₃),
64.3 (C-2), 68.0 (C-7), 71.0 (C-4), 73.0 (C-1), 76.5/76.6 (C-5 and C-6), 78.1 (C-3),
96.8/97.1/97.9 (OCH₂OCH₃), 101.2 [(CH₃)₂C].
2b: ¹³C NMR (CDCl₃) (chemical shift): 19.1/28.8 [(CH₃)₂C], 55.4/55.8/56.5 (OCH₂OCH₃),
64.6 (C-6), 68.0 (C-1), 69.6 (C-4), 73.1 (C-7), 73.5 (C-3), 76.5 (C-5), 77.2 (C-2),
96.8/97.3/98.2 (OCH₂OCH₃), 101.3 [(CH₃)₂C].
2c: ¹³C NMR (CDCl₃) (chemical shift): 19.2/28.8 [(CH₃)₂C], 55.5/55.8/56.5 (OCH₂OCH₃),
64.5 (C-6), 67.0 (C-1), 70.0 (C-4), 73.1 (C-7), 73.6 (C-3), 75.9 (C-2), 76.8 (C-5),
96.9/97.2/98.6 (OCH₂OCH₃), 101.4 [(CH₃)₂C].
2d: ¹³C NMR (CDCl₃) (chemical shift): 19.1/28.7 [(CH₃)₂C], 55.4/56.0/56.4 (OCH₂OCH₃),
64.3 (C-6), 66.9 (C-1), 71.1 (C-4), 73.0 (C-7), 75.8 (C-2), 76.1 (C-3), 77.9 (C-5),
96.7/96.9/97.9 (OCH₂OCH₃), 101.2 [(CH₃)₂C].

The results of measurement for mass analysis FAB (Fast Atom Bombardment)-MS and HR-FAB-MS of the resulting compounds (2a), (2b), (2c) and (2d) are indicated respectively as below:

TABLE 28

2a: FABMS m/z: 447 [M + H]⁺ (pos.), FABHRMS m/z:
447.1561 (C₁₆H₃₁O₁₂S₁ requires 447.1537).
2b: FABMS m/z: 447 [M + H]⁺ (pos.), FABHRMS m/z:
447.1545 (C₁₆H₃₁O₁₂S₁ requires 447.1537).
2c: FABMS m/z: 447 [M + H]⁺ (pos.), FABHRMS m/z:
447.1559 (C₁₆H₃₁O₁₂S₁ requires 447.1537).
2d: FABMS m/z: 447 [M + H]⁺ (pos.), FABHRMS m/z:
447.1549 (C₁₆H₃₁O₁₂S₁ requires 447.1537).

### Example 8

3,5-di-O-benzyl-1,2-O-isopropylidene-α-D-arabinofuranose (10b) (16.0 g, 43.2 mmol), obtained in the yield of 51% from D-arabinose through four steps was treated in substantially the same manner as in Example 1, thereby yielding a mixture (16.5 g; yield, 89% from 10b) of tert.-butyl (E)-5,7-di-O-benzyl-2,3-dideoxy-D-arabino-hepto-2-enoate (E-12b) and tert.-butyl(Z)-5,7-di-O-benzyl-2,3-dideoxy-D-arabino-hepto-2-enoate (Z-12b). This mixture was then recrystallized to give the compound (E-12b; 9.8 g, 53%). Further, a mixture (6.7 g; yield, 36%) of the compounds (E-12b) and (Z-12b) was obtained from the mother liquor. The results of measurement for melting points, specific rotatory power infrared absorption spectra, ¹H-NMR spectra, and ¹³C-NMR spectra are indicated respectively as below:

TABLE 29
E-10b: colorless needles (from n-hexane-diethyl ether). Mp 95-96° C. [α]_{D}²² +60.6 (c = 0.7,
CHCl3). IR (nujol): 3337, 1712, 1655, 1377, 1335, 1277, 1145, 1111, 1096 cm⁻¹.
E-10b: ¹H NMR (CDCl₃) (chemical shift): 1.49 (9H, s, (CH₃)₂C), 2.68 (d, J = 5.5 Hz, OH),
3.09 (d, J = 9.2 Hz, OH), 3.57 (dd, J = 9.8, 5.5 Hz, H-7a), 3.60 (dd, J = 8.3, 3.2 Hz, H-5),
3.65 (dd, J = 9.8, 3.5, Hz, H-7b), 3.92 (dddd, J = 8.3, 5.5, 5.5, 3.5 Hz, H-6), 4.488/
4.578 (each, d, J = 11.5, PhCH₂), 4.58 (1H, dddd, J = 9.2, 4.0, 3.2, 2.0 Hz, H-4), 4.492/
4.55 (each, d J = 11.8, PhCH₂), 6.10 (dd, J = 15.5, 2.0 Hz, H-2), 7.02 (dd, J = 15.5, 4.0 Hz,
H-3), 7.20-7.38 (10H, m, arom.).
Z-10b: ¹H NMR (CDCl₃) (chemical shift): 1.46 (9H, s, (CH₃)₂C), 3.14 (1H, d, J = 5.5, OH),
3.61-3.67 (1H, m, H-7a), 3.71 (1H, dd, J = 6.9, 2.9 Hz, H-5), 3.72 (1H, dd, J = 9.8, 3.7 Hz,
H-7b), 4.00 (1H, d, J = 6.6 Hz, OH), 4.05 (1H, dddd, J = 7.1, 6.9, 5.5, 3.7 Hz, H-6),
4.53 (1H, d, J = 11.8 Hz, PhCH₂), 4.55-4.58 (3H, m, PhCH₂), 5.28 (1H, dddd, J = 6.9, 6.6,
2.9, 1.7 Hz, H-4) 5.77 (1H, dd, J = 12.0, 1.7 Hz, H-2), 6.29 (1H, dd, J = 12.0, 6.9 Hz,
H-3), 7.21-7.37 (10H, m, arom.).
E-10b: ¹³C NMR (CDCl₃) (chemical shift) δ: 28.1 [(CH₃)₃C], 70.6 (C-4), 70.66 (C-7),
70.69 (C-6), 73.5/73.8 (PhCH₂), 79.4 (C-5), 81.4 [(CH₃)₃C], 123.5 (C-2), 127.9/128.0/128.1/
128.47/128.52 (d, arom.), 137.38/137.5 (s, arom.), 146.5 (C-3), 165.6 (C-1).
Z-10b: ¹³C NMR (CDCl₃) (chemical shift): 28.1 [(CH₃)₂C], 68.3 (C-4), 70.9 (C-6),
71.1 (C-7), 73.4/73.7 (PhCH₂), 80.3 (C-5), 81.3 [(CH₃)₂C], 122.6 (C-2), 127.7/127.8/127.9/
128.38/128.40 (d, arom.), 137.8/138.0 (s, arom.), 148.6 (C-3), 166.0 (C-1).

### Example 9

The compound (E-12b) (4.86 g, 11.4 mmol), obtained by Example 8, was treated in substantially the same manner as in Example 2, thereby yielding an oily material in the amount of 5.32 g. A small amount of the oily material was then purified by column chromatography to give tert.-butyl (E)-5,7-di-O-benzyl-2,3-dideoxy-4,6-O-isopropylidene-D-arabino-hepto-2-enoate (E-13b-1) as a sample for analysis. The results of measurement for ¹H-NMR spectrum and ¹³C-NMR spectrum of the resulting compound (E-13b-1) are indicated as below:

### Example 30

E-13b-1: ¹H NMR (CDCl₃) (chemical shift): 1.36/L45 (each 3H, s, (CH₃)₂C), 1.48 (9H, s, (CH₃)₃C), 3.51 (1H, dd, J=10.5, 4.5 Hz, H-7a), 3.53 (1H, dd, J=10.5, 4.0 Hz, H-7b) 3.75 (1H, dd, J=7.0, 3.8 Hz, H-5) 3.87 (1H, ddd, J=7.0, 4.5, 4.0 Hz, H-6), 4.26/4.46 (each 1H, d, J=11.2 Hz, PhCH₂) 4.49/4.57 (each 1H, d, J=12.2 Hz, PhCH₂), 4.54 (1H, ddd, J=5.2, 3.8, 1.7 Hz, H-4) 6.09 (1H, dd, J=15.6, 1.7 Hz, H-2) 6.95 (1H, dd, J=15.6, 5.2 Hz, H-3) 7.16-7.37 (10H, m, arom.).

E-13b-1: ¹³C NMR (CDCl₃) (chemical shift): 23.9/24.9 [(CH₃)₂C], 28.1 [(CH₃)₃C], 69.8 (C-7), 71.2 (C-4), 72.2 (C-6), 73.3/73.5 (PhCH_{2l ),} 78.1 (C-5), 80.3 [(CH₃)₃C], 101.3 [(CH₃)₂C], 124.3 (C-2), 127.7/127.85/127.92/128.26/128.31/128.4 (d, arom.), 137.5/138.0 (s, arom.), 141.9 (C-3), 165.4 (C-1).

### Example 10

The compound (E-13b-1) (5.3 g), obtained by Example 9, was treated in substantially the same manner as in Example 3 to give (E)-5,7-di-O-benzyl-2,3-dideoxy-4,6-O-isopropylidene-D-arabino-hepto-2-enitol (E-14b-1) in the amount of 3.8 g. The resulting compound was then treated in substantially the same manner as in Examples 4 and 5 to give 5,7-di-O-benzyl-4,6-O-isopropylidene-1,2,3-tri-O-methoxymethyl-D-glycero-D-galacto-heptitol (16e-1) (2.68 g; yield, 59% from E-10b) and 1,3-di-O-benzyl-2,4-O-isopropylidene-5,6,7-tri-O-methoxymethyl-D-glycero-L-allo-heptitol (16f-1) (0.37 g; yield, 8% from E-10b). The results of measurement for ¹H-NMR and ¹³C-NMR spectra of the compound 16e-1 are indicated respectively as below:

TABLE 31
16e-1: ¹H NMR (CDCl₃) (chemical shift): 1.35/1.45 (each 3H, s, (CH₃)₂C), 3.34/3.37/
3.38 (each 3H, s, OCH₂OCH₃), 3.52 (1H, dd, J = 10.1, 5.3 Hz, H-7a), 3.58 (1H, dd, J = 10.1,
5.9 Hz, H-7b), 3.69 (1H, dd, J = 9.8, 8.0 Hz, H-1a), 3.780 (1H, dd, J = 4.1, 2.4 Hz, H-5),
3.782 (1H, dd, J = 9.8, 6.0 Hz, H-1b), 3.98 (1H, ddd, J = 8.0, 6.0, 1.4 Hz, H-2), 4.03 (1H,
dd, J = 9.3, 2.4 Hz, H-4), 4.08 (1H, dd, J = 9.3, 1.4 Hz, H-3), 4.13 (1H, ddd, J = 5.9, 5.3,
4.1 Hz, H-6), 4.41/4.55 (each 1H d, J = 11.5 Hz, PhCH₂), 4.56/4.59 (each 1H, d, J = 12.2 Hz,
PhCH₂), 4.61/4.62 (each 1H, d, J = 5.5 Hz, OCH₂OCH₃), 4.63/4.67 (each 1H, d, J = 6.5 Hz,
OCH₂OCH₃), 4.74/4.79 (each 1H, d, J = 6.7 Hz), 7.24-7.36 (10H, m, arom.).
16e-1: ¹³C NMR (CDCl₃) (chemical shift): 24.1/26.7 [(CH₃)₂C], 55.4/55.7/
56.2 (OCH₂OCH₃), 67.6 (C-1), 68.8 (C-4), 71.3 (C-7), 71.4/73.4 (PhCH₂), 73.2 (C-6),
75.5 (C-5), 76.5 (C-3), 76.7 (C-2), 96.8/98.4/98.8 (OCH₂OCH₃), 100.6 [(CH₃)₂C],
127.5/127.7/127.8/128.3/128.4 (d, arom.), 138.0/138.4 (s, arom.).

### Example 11

The compound 16e-1 (2.86 g, 5.07 mmol), obtained by Example 10, was treated in accordance with Example 6 to give 4,6-O-isopropylidene-1,2,3-tri-O-methoxymethyl-D-glycero-D-galacto-heptitol (17e-1; 1.81 g; yield, 93%). The results of measurement for ¹H-NMR and ¹³C-NMR spectra of the compound 17e-1 are indicated respectively as below:

TABLE 32
17e-1: ¹H NMR (CDCl₃) (chemical shift): 1.33/1.42 (each 3H, s,
(CH₃)₂C), 1.98 (1H, br s, OH), 3.375/3.377/3.46 (each 3H, s,
OCH₂OCH₃), 3.62 (1H, dd, J = 9.2, 9.2 Hz, H-1a), 3.68 (1H, ddd,
J = ca. 6.6, 6.6, 3.2 Hz, H-6), 3.69-3.75 (1H, br-m, H-7a), 3.83 (1H, dd,
J = 9.2, 5.5 Hz, H-1b), 3.84-3.89 (1H, br-m, H-7b), 3.92 (1H, ddd,
J = 9.2, 5.5, 1.7 Hz H-2), 3.95 (1H, dd, J = 9.7, 3.4 Hz, H-4), 3.98
(1H, ddd, J = 3.7, 3.4, 3.2 Hz H-5), 4.16 (1H, dd, J = 9.7, 1.7 Hz H-3),
4.40 (1H, d, J = 3.7 Hz, OH), 4.62/4.64 (each 1H, d, J = 6.6 Hz
OCH₂OCH₃), 4.71/4.76 (each 1H, d, J = 6.9 Hz OCH₂OCH₃),
4.77/4.96 (each 1H, d, J = 6.9 Hz OCH₂OCH₃).
17e-1: ¹³C NMR (CDCl₃) (chemical shift): 24.0/24.6 [(CH₃)₂C],
55.7/55.8/56.6 (OCH₂OCH₃), 63.7 (C-7), 66.5 (C-1), 68.3 (C-5), 69.3
(C-4), 73.9 (C-6), 76.9 (C-3), 77.1 (C-2), 97.0/98.7/99.4 (OCH₂OCH₃),
101.2 [(CH₃)₂C].

### Example 12

The compound 17e-1 (711 mg, 1.9 mmol), obtained by Example 11, was treated in accordance with Example 7 to give 4,6-O-isopropylidene-1,2,3-tri-O-methoxymethyl-D-glycero-D-galacto-heptitol 5,7-cyclosulfate (2e-1) (68.6 g; yield, 8%). The result of measurement for ¹³C-NMR spectrum of the compound 2e-1 is indicated as below:

TABLE 33
2e-1: ¹³C NMR (CDCl₃) (chemical shift): 23.0/25.8 [(CH₃)₂C],
55.6/55.8/56.4 (OCH₂OCH₃), 62.0, 66.6, 67.4, 72.9, 75.5, 76.6, 82.0,
96.9/98.2/98.9 (OCH₂OCH₃), 103.3 [(CH₃)₂C].

### Example 13

The compound E-12b (9.2 g 21.5 mmol) obtained by Example 8 was treated in accordance with Example 5 to give an oily material in the amount of 11.6 g. A small amount of the resulting oily material was purified by column chromatography to give tert.-butyl (E)-5,7-di-O-benzyl-2,3-dideoxy-4,6-di-O-methoxymethyl-D-arabino-hepto-2-enoate (E-13b-2) as a sample for analysis. The results of measurement for specific rotatory power, infrared absorption spectrum, ¹H-NMR spectrum, ¹³C-NMR spectrum and mass analysis FAB (Fast Atom Bombartmemt)-MS and HR-FAB-MS of the resulting compound (E-13b-2) are indicated as below:

TABLE 34
E-13b-2: Colorless oil [α]_{D}²⁴ −19.4 (c = 1.00, CHCl₃). IR (CHCl₃): 1713, 1654, 1454,
1365, 1307, 1253, 1211, 1153, 1103, 1038 cm⁻¹.
E-13b-2: ¹H NMR (CDCl₃) (chemical shift): 1.48 (9H, s, (CH₃)₃C), 3.34/3.36 (1H, s,
OCH₂OCH₃), 3.73 (1H, dd, J = 10.3, 4.4 Hz, H-7a), 3.76 (1H, dd, J = 6.0, 4.0, H-5),
3.79 (1H, dd, J = 10.3, 3.2 Hz, H-7b), 3.91 (1H, ddd, J = 6.0, 4.4, 3.2 Hz, H-6), 4.46 (1H, ddd,
J = 6.3, 4.0, 1.2 Hz, H-4), 4.52/4.54 (each 1H, d, J = 12.0 Hz, PhCH₂), 4.59/4.63 (each
1H, d, J = 11.2 Hz, PhCH₂), 4.62/4.63 (each 1H, d, J = 6.9 Hz, OCH₂OCH₃), 4.73/
4.75 (each 1H, d, J = 6.9 Hz, OCH₂OCH₃), 5.98 (1H, dd, J = 15.8, 1.2 Hz, H-2), 6.86 (1H, dd,
J = 15.8, 6.3, H-3), 7.24-7.35 (10H, m, arom.).
E-13b-2: ¹³C NMR (CDCl₃) (chemical shift): 28.1 [(CH₃)₃C], 55.7/56.1 (OCH₂OCH₃),
69.6 (C-7), 73.4/74.7 (PhCH₂), 75.7 (C-4), 76.9 (C-6), 80.5 [(CH₃)₃C], 80.7 (C-5), 95.4/
96.9 (OCH₂OCH₃), 124.9 (C-2), 127.6/127.7/127.9/128.2/128.26/128.34 (d, arom.),
137.8/138.0 (s arom.), 144.2 (C-3), 165.2 (C-1).
E-13b-2: FABMS m/z: 517 [M + H]⁺ (pos.), FABHRMS m/z: 517.2818 (C₂₉H₄₁O₈ requires
517.2801).

### Example 14

The compound E-13b-2 (11.1 g) obtained by Example 13 was treated in accordance with Example 3 to give an oily material in the amount of 9.63 g. A small amount of the resulting oily material was purified by column chromatography to give (E)-5,7-di-O-benzyl-2,3-dideoxy-4,6-di-O-methoxymethyl-D-arabino-hepto-2-enitol (E-14b-2) as a sample for analysis. The results of measurement for specific rotatory power, infrared absorption spectrum, ¹H-NMR spectrum, ¹³C-NMR spectrum and mass analysis FAB (Fast Atom Bombartmemt)-MS and HR-FAB-MS of the resulting compound (E-14b-2) are indicated as below:

TABLE 35
E-14b-2: colorless oil. [α]_{D}²⁴ −38.5 (c = 1.00, CHCl₃). IR (neat): 3456, 1454, 1365,
1211, 1153, 1099, 1030 cm⁻¹.
E-14b-2: ¹H NMR (CDCl₃) (chemical shift): 1.50 (1H, br s, OH), 3.33/3.37 (each 3H, s, OCH₂CH₃),
3.70 (1H, dd, J = 5.2, 4.6, H-5), 3.74 (1H, dd, J = 10.3, 5.2 Hz, H-7a), 3.80 (1H, dd, J = 10.3,
2.9 Hz, H-7b), 3.94 (1H, ddd, J = 5.2, 5.2, 2.9 Hz, H-6), 4.03 (1H, br d, J = ca. 5.2 Hz, H-1), 4.24
(1H, br dd, J = 7.9, 4.6 Hz, H-4), 4.52/4.58 (each 1H, d, J = 11.7 Hz,
PhCH₂), 4.57/4.67 (each 1H, d, J = 6.9 Hz, OCH₂OCH₃), 4.63/4.72 (each 1H, d,
J = 11.5 Hz, PhCH₂), 4.74/4.75 (each 1H, d, J = 6.9 Hz, OCH₂OCH₃), 5.57 (1H, ddt,
J = 15.8, 7.9, 1.4 Hz, H-3), 5.80 (1H, dddd, J = 15.8, 5.2, 0.6, Hz, H-2), 7.13-7.35 (10H, m, arom.).
E-14b-2: ¹³C NMR (CDCl₃) (chemical shift): 55.6/55.8 (OCH₂OCH₃), 62.7 (C-1), 69.8 (C-7),
73.4/74.6 (PhCH₂), 76.5 (C-4), 77.0 (C-6), 81.4 (C-5), 94.3/96.7 (OCH₂OCH₃), 127.6/127.7/127.9/
128.2/128.30/128.33 (d, arom.), 128.4 (C-2), 133.5 (C-3), 138.1/138.4 (s, arom.).
E-14b-2: FABMS m/z: 447 [M + H]⁺ (pos.), FABHRMS m/z: 447.2381 (C₂₅H₃₅O₇ requires
447.2383).

### Example 15

The compound E-14b-2 (9.5 g) obtained by Example 14 was treated in substantially the same manner as in Example 4 to give a mixture (10.4 g) of 5,7-di-O-benzyl-4,6-di-O-methoxymethyl-D-glycero-D-galacto-heptitol (15e-2) and 1,3-di-O-benzyl-2,4-di-O-methoxymethyl-D-glycero-L-allo-heptitol (16f-2). The resulting mixture was then treated in accordance with Example 5 to give 5,7-di-O-benzyl-1,2,3,4,6-penta-O-methoxymethyl-D-glycero-D-galacto-heptitol (16e-2) (7.99 g; yield, 61% from E-12b) and 1,3-di-O-benzyl-2,4,5,6,7-penta-O-methoxymethyl-D-glycero-L-allo-heptitol (16f-2) (2.64 g; yield, 20% from E-12b), respectively. The results of measurement for specific rotatory power, infrared absorption spectrum, ¹H-NMR spectrum, ¹³C-NMR spectrum and mass analysis FAB (Fast Atom Bombartmemt)-MS and HR-FAB-MS of the resulting compounds (16e-2) and (16f-2) are indicated as below:

TABLE 36
16e-2: Colorless oil [α]_{D}²⁴ −9.4 (c = 1.32, CHCl3). IR (neat): 1454, 1365, 1211, 1153, 1103, 1034 cm⁻¹.
16f-2: Colorless oil [α]_{D}²⁴ +14.2 (c = 0.99, CHCl₃). IR (neat): 1454, 1366, 1211, 1153, 1103, 1030 cm⁻¹.
16e-2: ¹H NMR (CDCl₃) (chemical shift): 3.35/3.36/3.369/3.373/3.40 (each 3H, s, OCH₂OCH₃), 3.72 (1H,
dd, J = 10.3, 5.2 Hz, H-7a), 3.75 (2H, d-like, J = ca 4.6 Hz, H-1), 3.84 (1H, dd, J = 10.3, 3.4 Hz, H-7b),
3.95 (1H, t-like, J = 4.8 Hz, H-5), 3.99-4.04 (4H, m, H-2, H-3, H-4, H-6), 4.51/4.54 (each 1H, d,
J = 12.1 Hz, PhCH₂), 4.62/4.63 (each 1H, d, J = 6.9 Hz, OCH₂OCH₃), 4.71/4.80
(each 1H, d, J = 6.6 Hz, OCH₂OCH₃), 4.70-4.73 (3H, m, OCH₂OCH₃), 4.74 (2H, s, PhCH₂),
4.74-4.78 (3H, m OCH₂OCH₃), 7.23-7.35 (10H, m, arom).
16f-2: ¹H NMR (CDCl₃) (chemical shift): 3.33/3.369/3.375 (each 3H, s, OCH₂OCH₃), 3.40 (6H, s,
OCH₂OCH₃), 3.68 (1H, dd, J = 10.6, 4.2 Hz, H-7a), 3.73 (1H, dd, J = 10.6, 5.3 Hz, H-7b), 3.76 (1H, dd,
J = 10.4, 5.6 Hz, H-1a), 3.87 (1H, dd, J = 10.4, 3.4 Hz, H-1b), 3.87-3.90 (1H, m, H-6), 3.90 (1H, dd,
J = 5.2, 5.2 Hz, H-5), 3.91 (1H, dd, J = 5.2, 5.2 Hz, H-3), 3.99 (1H, dd, J = 5.2, 5.2 Hz, H-4), 4.04-4.02
(1H, ddd, J = 5.6, 5.2, 3.4 Hz, H-2), 4.54 (2H, s, PhCH₂), 4.57/4.59 (each 1H, d, J = 6.4 Hz, OCH₂OCH₃),
4.65/4.75 (each 1H, d, J = 11.2 Hz, PhCH₂), 4.71-4.83 (9H, m, OCH₂OCH₃ including one proton doublet
(J = 11.2 Hz) due to PhCH₂ at δ 4.75), 7.25-7.35 (10H, m, arom.).
16e-2: ¹³C NMR (CDCl₃) (chemical shift): 55.3/55.5/55.8/55.9/56.0 (OCH₂OCH₃), 68.0 (C-1), 69.8 (C-7),
73.3/74.1 (PhCH₂), 76.8, 77.1 (2 cabons), 77.5 (C-2, C-3, C-4, C-6), 79.1 (C-5), 96.2/96.8/97.15/97.19/97.5
(OCH₂OCH₃), 127.3/127.5/127.7/127.8/128.2/128.3 (d, arom.), 138.2/138.8 (s, arom).
16f-2: ¹³C NMR (CDCl₃) (chemical shift): 55.3/55.6/55.9/56.3/56.4 (OCH₂OCH₃), 67.8 (C-7), 70.2 (C-1),
73.3/74.4 (PhCH₂), 76.6 (C-6), 77.1 (C-4), 77.6 (C-2, C-5), 79.1 (C-3), 96.7/96.8/97.1/98.6/93.7
(OCH₂OCH₃), 127.5/127.7/128.0/128.26/128.3 (d, arom.), 138.2/138.4 (s, arom.).
16e-2: FABMS m/z: 613 [M + H]⁺ (pos.), FABHRMS m/z: 613.3243 (C₃₁H₄₉O₁₂ requires 613.3224).
16f-2: FABMS m/z: 613 [M + H]⁺ (pos.), FABHRMS m/z: 613.3238 (C₃₁H₄₉O₁₂ requires 613.3224).

### Example 16

The compound 16e-2 (3.06 g, 5.0 mmol) obtained by Example 15 was treated in substantially the same manner as in Example 6 leading to 1,2,3,4,6-penta-O-methoxymethyl-D-glycero-D-galacto-heptol (17e-2) (yield, 96%). The results of measurement for specific rotatory power, infrared absorption spectrum, ¹H-NMR spectrum and ¹³C-NMR spectrum of the resulting compound (17e-2) are indicated as below:

TABLE 37
17e-2: Colorless oil. [α]D24 −31.0 (c = 1.40, CHCl3). IR (neat): 3472, 1465, 1443,
1407, 1384, 1215, 1153, 1099, 1026 cm⁻¹.
17e-2: ¹H NMR (CDCl₃) (chemical shift): 3.25 (1H, dd J = 7.2, 6.0 Hz, OH), 3.37/
3.41 (each 3H, s, OCH₂OCH₃), 3.43 (6H, s, OCH₂OCH₃), 3.45 (3H, s, OCH₂OCH₃), 3.63 (1H, d,
J = 5.0 Hz, OH), 3.67 (1H, ddd, J = 8.8, 5.3, 3.3 Hz, H-6), 3.70 (1H, dd, J = 10.6, 5.2 Hz,
H-1a), 3.74 (1H, dd, J = 10.6, 5.0 Hz, H-1b), 3.75 (1H, ddd, J = 11.8, 6.0, 5.3 Hz, H-7a),
3.85 (1H, br-dd, J = 8.8, 5.0 Hz, H-5), 3.95 (1H, ddd, J = 5.2, 5.2, 5.0 Hz, H-2),
3.96 (1H, ddd, J = 11.8, 7.2, 3.3 Hz, H-7b), 4.05 (1H, dd, J = 5.2, 5.2 Hz, H-3), 4.06 (1H,
br-d, J = 5.2 Hz, H-4), 4.64 (2H, s-like, OCH₂OCH₃), 4.70-4.86 (8H, m, OCH₂OCH₃).
17e-2: ¹³C NMR (CDCl₃) (chemical shift): 55.5/55.8/55.9/56.2/56.3 (OCH₂OCH₃),
63.5 (C-7), 67.3 (C-1), 70.5 (C-5), 75.4 (C-4), 76.7 (C-2), 78.7 (C-3), 80.6 (C-6),
96.9/97.1/97.20/97.24/98.5 (OCH₂OCH₃).

### Example 17

The compound 17e-2 (304 mg, 0.7 mmol) obtained by Example 16 was treated in accordance with Example 7 to give 1,2,3,4,6-penta-O-methoxymethyl-D-glycero-D-galacto-heptol 5,7-cyclosulfate (2e-2) (337 mg; yield, 97%). The results of measurement for infrared absorption spectrum, ¹H-NMR spectrum and ¹³C-NMR spectrum of the resulting compound (2e-2) are indicated as below:

TABLE 38
3e-2: Colorless oil. IR (neat): 2947, 2897, 2827, 1470, 1447, 1404, 1204, 1153, 1110,
918 cm⁻¹.
3e-2: ¹H NMR (CDCl₃) (chemical shift): 3.37/3.38/3.40/3.44/3.45 (each 3H, s,
OCH₂OCH₃), 3.73 (1H, dd, J = 10.0, 7.0 Hz, H-1a), 3.82 (1H, dd, J = 10.0, 5.8 Hz, H-1b),
3.87 (1H, dd, J = 8.6, 1.8, H-3), 3.95 (1H, ddd, J = 7.0, 5.8, 1.8 Hz, H-2), 4.16 (1H, dd,
J = 8.6, 0.8 Hz, H-4), 4.31 (1H, ddd, J = 10.2, 10.0, 5.4 Hz, H-6), 4.55 (1H, dd, J = 11.0,
10.2 Hz, H-7a), 4.63/4.86 (each 1H, d, J = 7.0 Hz, OCH₂OCH₃), 4.64/4.66 (each 1H, d, J = 6.4 Hz,
OCH₂OCH₃₎, 4.73/4.81 (each 1H, d, J = 6.8 Hz, OCH₂OCH₃), 4.74/4.82 (each
1H, d, J = 6.8 Hz, OCH₂OCH₃), 4.75/4.77 (each 1H, dd, J = 6.4 Hz, OCH₂OCH₃), 5.01 (1H,
dd, J = 10.0, 0.8 Hz, H-5).
¹³C NMR (175 MHz, CDCl₃). δ: 55.5/55.9/56.1/56.2/56.4 (OCH₂OCH₃), 67.4 (C-1),
6.76 (C-6), 73.0 (C-7), 75.9 (C-2), 76.3 (C-4), 76.5 (C-3), 83.4 (C-5), 96.9/97.3/97.8/98.9/
99.0 (OCH₂OCH₃).

### Example 18

D-xylose was treated in accordance with reactions (i) and (ii) as illustrated in [Chemical Formula 7] above and then benzylated to give 3,5-di-O-benzyl-1,2-O-iso-propylidene-α-D-xylofuranose (10c) (23.0 g, 62 mmol) which in turn was treated in substantially the same manner as in Example 1 to give a mixture (23.1 g; yield, 87% from 10c) of tert.-butyl-(E)-5,7-di-O-benzyl-2,3-dideoxy-D-xylo-hepto-2-enoate (E-12c) and tert.-butyl-(Z)-5,7-di-O-benzyl-2,3-dideoxy-D-xylo-hepto-2-enoate (Z-12c). This mixture was recrystallized yielding the compound E-12c (14.6g, 55%). Further, a mixture of the mixture compounds E-12c and Z-12c was obtained from the mother liquor in the yield of 8.6 g (yield, 32%). A small amount of this mixture was purified by column chromatography to give the compound Z-12c as a sample for analysis. The results of measurement for melting points, specific rotatory power, infrared absorption spectrum, ¹H-NMR spectrum, ¹³C-NMR spectrum as well as mass analysis FAB (Fast Atom Bombartmemt)-MS and HR-FAB-MS of the resulting compounds (E-12d) and (Z-12c) are indicated respectively as below:

TABLE 39
E-12c: Colorless needles (from hexane-AcOEt). Mp. 86-87° C. [α]_{D}²⁴ −54.2 (c = 1.07, CHCl₃). IR
(nujol): 3364, 1709, 1655, 1281, 1153, 1138, 1130, 1103 cm⁻¹.
Z-12c: Colorless oil. [α]_{D}²⁴ −78.4 (c = 1.78, CHCl₃). IR (neat): 3418, 1747, 1651, 1601, 1454, 1392,
1161, 1092 cm⁻¹.
E-12c: ¹H NMR (CDCl₃) (chemical shift): 1.49 [9H, s, (CH₃)₃C], 2.65 (1H, br d, J = 5.7 Hz, OH),
2.99 (1H, br d, J = 6.0 Hz, OH), 3.53 (1H, dd, J = 9.8, 5.7 Hz, H-7a), 3.594 (1H, dd, J = 9.8, 5.7 Hz,
H-7b), 3.597 (1H, dd, J = 5.7, 4.3 Hz, H-5), 3.95 (1H, dddd, J = 5.7, 5.7, 5.7, 4.3 Hz, H-6), 4.47
(1H, dddd, J = 6.0, 4.6, 4.3, 1.9 Hz, H-4), 4.50/4.52 (each 1H, d, J = 11.9 Hz, PhCH₂), 4.58/4.64
(each 1H, d, J = 11.2 Hz, PhCH₂), 6.06 (1H, dd, J = 15.7, 1.9 Hz, H-2), 6.90 (1H, dd, J = 15.7,
4.6 Hz, H-3), 7.25-7.37 (10H, m, arom.).
Z-12c: ¹H NMR (CDCl₃) (chemical shift): 1.47 [9H, 5, (CH₃)₃C], 3.22 (1H, br s, OH), 3.58 (1H,
dd, J = 9.7, 6.1 Hz, H-7a), 3.61 (1H, dd, J = 9.7, 6.1 Hz, H-7b), 3.67 (1H, dd, J = 4.9, 3.2 Hz, H-5),
3.84 (1H, br s, OH) 4.03 (1H, ddd, J = 6.1, 6.1, 3.2 Hz, H-6), 4.50/4.55 (each 1H, d, J = 11.8 Hz,
PhCH₂), 4.64/4.67 (each 1H, d, J = 11.3 Hz, PhCH₂), 5.18 (1H, ddd, J = 7.2, 4.9, 1.5 Hz, H-4)
5.79 (1H, dd, J = 12.0, 1.5 Hz, H-2), 6.26 (1H, dd, J = 12.0, 7.2 Hz, H-3), 7.26-7.36 (10H, m,
arom.).
E-12c: ¹³C NMR (CDCl₃) (chemical shift): 28.1 [(CH₃)₃C], 70.81 (C-7), 70.83 (C-6), 71.2 (C-4),
73.5/74.8 (PhCH₂), 80.4 [(CH₃)₃C], 80.9 (C-5), 123.6 (C-2), 127.9/128.1/128.2/128.48/128.50
(d, arom.), 137.4/137.5 (s, arom.), 145.7 (C-3), 165.5 (C-1).
Z-12c: ¹³C NMR (CDCl₃) (chemical shift): 28.0 [(CH₃)₃C], 69.2 (C-4), 70.7 (C-6), 71.0 (C-7),
73.4/74.8 (PhCH₂), 80.7 (C-5), 81.5 [(CH₃)₃C], 122.9 (C-2), 127.7/127.87/127.92/128.2/128.38/
128.42 (d, arom.), 137.89/137.92 (s, arom.), 148.0 (C-3), 166,4 (C-1).
E-12c: FABMS m/z: 429 [M + H]⁺ (pos.), FABHRMS m/z: 429.2277 (C₂₅H₃₃O₆ requires 429.2278).
Z-12c: FABMS m/z: 429 [M + H]⁺ (pos.), FABHRMS m/z: 429.2256 (C₂₅H₃₃O₆ requires 429.2278).

### Example 19

The compound E-12c (14.5 g, 33.9 mmol) obtained by Example 18 was treated in substantially the same manner as in Example 2 to give an oily material in the amount of 16 g. A small amount of the resulting oily material was purified by column chromatography, thereby resulting in the formation of tert.-butyl (E)-5,7-di-O-benzyl-2,3-dideoxy-4,6-O-isopropylidene-D-xylo-hepto-2-enoate (E-13c) as a sample for analysis. The results of measurement for melting point, specific rotatory power, infrared absorption spectrum, ¹H-NMR spectrum, ¹³C-NMR spectrum as well as mass analysis FAB (Fast Atom Bombartmemt)-MS and HR-FAB-MS of the resulting compound (E-13c) are indicated as below:

TABLE 40
E-13c: Mp 89-91° C. [α]_{D}²⁴ −31.6 (c = 4.40, CHCl₃). IR (nujol):
1701, 1651, 1304, 1200, 1157, 1092, 1045, 1026 cm⁻¹.
E-13c: ¹H NMR (CDCl₃) (chemical shift): 1.45/1.48 [each 3H, s,
(CH₃)₂C], 1.47 [9H, s, (CH₃)₃C], 3.44 (1H, dd, J = 1.7, 1.7 Hz, H-5),
3.53 (1H, dd, J = 9.1, 5.3 Hz, H-7a), 3.63 (1H, dd, J = 9.1, 7.5 Hz, H-7b),
4.15 (1H, ddd, J = 7.5, 5.3, 1.7 Hz, H-6), 4.45/4.51 (each 1H, d, J =
11.7 Hz, PhCH₂), 4.50/4.56 (each 1H, d, J = 11.3 Hz, PhCH₂), 4.52 (1H,
ddd, J = 4.6, 1.7, 1.7 Hz, H-4), 6.06 (1H, dd, J = 15.6, 1.7 Hz, H-2), 6.81
(1H, dd, J = 15.6, 4.6 Hz, H-3), 7.23-7.36 (10H, arom.).
E-13c: ¹³C NMR (CDCl₃) (chemical shift): 19.0/29.5 [(CH₃)₂C],
28.1 [(CH₃)₃C], 69.2 (C-7), 71.4 (C-6), 71.7 (C-5), 72.1 (C-4), 73.6/74.3
(PhCH₂), 80.3 [(CH₃)₃C], 99.1 [(CH₃)₂C], 123.9 (C-2), 127.7/127.8/
127.9/128.2/128.4 (d, arom.), 137.78/137.82 (s, arom.), 143.1 (C-3),
165.5 (C-1).
FABMS m/z: 469 [M + H]⁺ (pos.), FABHRMS m/z: 469.2618 (C₂₈H₃₇O₆
requires 469.2590).

### Example 20

The compound E-13c (16 g) obtained by Example 19 was treated in substantially the same manner as in Example 3 to give (E)-5,7-di-O-benzyl-2,3-dideoxy-4,6-O-isopropylidene-D-xylo-hepto-2-enitol (E-14c) (13.2 g; yield, 92%). The results of measurement for specific rotatory power, infrared absorption spectrum, ¹H-NMR spectrum, ¹³C-NMR spectrum as well as mass analysis FAB (Fast Atom Bombartmemt)-MS and HR-FAB-MS of the resulting compound (E-13c) are indicated as below:

TABLE 41
E-14c: Colorless oil. [α]_{D}²⁴ −38.8, (c = 1.24, CHCl₃). IR (neat): 3418,
1497, 1381, 1265, 1204, 1169, 1103, 1069, 1026 cm⁻¹.
E-14c: ¹H NMR (CDCl₃) (chemical shift): 1.23 (1H, br s, OH), 1.47 [6H,
s, (CH₃)₂C], 3.36 (1H, dd, J = 1.7, 1.7 Hz, H-5), 3.54 (1H, dd, J =
9.1, 5.3 Hz, H-7a), 3.66 (1H, dd, J = 9.1, 7.7 Hz, H-7b), 3.99 (1H, br
dd-like, J = ca. 11.5, 5.3 Hz, H-1a), 4.04 (1H, br dd-like, J = ca. 11.5,
5.3 Hz, H-1b), 4.14 (1H, ddd, J = 7.7, 5.3, 1.7 Hz, H-6), 4.35 (1H,
ddd, J = 6.5, 1.7, 1.0 Hz, H-4), 4.47/4.53 (each 1H, d, J = 11.9 Hz,
PhCH₂), 4.54/4.63 (each 1H, d, J = 11.9 Hz, PhCH₂), 5.6 (1H, dddd,
J = 15.6, 6.5, 1.6, 1.6 Hz, H-3), 5.85 (1H, dddd, J = 15.6, 5.3, 5.3,
1.0 Hz, H-2), 7.26-7.36 (10H, m, arom)
E-14c: ¹³C NMR (150 MHz, CDCl₃) (chemical shift): 19.1/29.6
[(CH₃)₂C], 63.0 (C-1), 69.1 (C-7), 71.3 (C-6), 72.4 (C-5), 73.1 (C-4),
73.5/74.4 (PhCH₂), 98.9 [(CH₃)₂C], 127.7/127.8/127.9/128.2/128.4/128.6
(d, arom), 128.7 (C-3), 131.7 (C-2), 137.8/138.3 (s arom).
FABMS m/z: 399 [M + H]⁺ (pos.), FABHRMS m/z: 399.2189
(C₂₄H₃₁O₅ requires 399.2171).

### Example 21

The compound E-14c (12.4 g, 31.2 mmol) obtained by Example 20 was treated in substantially the same manner as in Example 4 to give a mixture (13.2 g) of 5,7-di-O-benzyl-4,6-O-isopropylidene-D-glycero-L-galacto-heptitol (15g) and 5,7-di-O-benzyl-4,6-O-isopropylidene-meso-glycero-yd-heptitol (15h). The mixture was then treated in substantially the same manner as in Example 5 yielding a mixture of 5,7-di-O-benzyl-4,6-O-isopropylidene-1,2,3-tri-O-methoxymethyl-D-glycero-L-galacto-heptitol (16g) (9.95 g; yield, 56% from E-14c) and 1,3-di-O-benzyl-2,4-O-isopropylidene-5,6,7-tri-O-methoxymethyl-meso-glycero-yd-heptitol (16h) (2.9 g; yield, 17% from E-14c). The results of measurement for specific rotatory power, infrared absorption spectrum, ¹H-NMR spectrum, ¹³C-NMR spectrum as well as mass analysis FAB (Fast Atom Bombartmemt)-MS and HR-FAB-MS of the resulting compounds (16g) and (16h) are indicated respectively as below:

TABLE 42
13i: Colorless oil. [α]_{D}²⁴ −14.0 (c = 1.34, CHCl₃). IR (neat): 1454, 1381, 1265, 1204,
1153, 1103, 1034 cm⁻¹.
13j: Colorless oil. [α]_{D}²⁴ +7.6 (c = 1.13, CHCl₃). IR (neat): 1454, 1381, 1265, 1204,
1153, 1103, 1026 cm⁻¹.
13i: ¹H NMR (CDCl₃) (chemical shift): 1.14/1.45 (each 3H, s, (CH₃)₂C), 3.32/3.38/
3.40 (each 3H, s, OCH₂OCH₃), 3.58 (1H, dd, J = 9.5, 5.8 Hz, H-7a), 3.68 (1H, dd,
J = 9.6, 7.7 Hz,
H-1a), 3.69 (1H, dd, J = 9.5, 7.2 Hz, H-7b), 3.71 (1H, dd, J = 1.4, 1.4 Hz, H-5),
3.75 (1H, dd, J = 9.6, 6.0 Hz, H-1b), 4.00 (1H, ddd, J = 7.7, 6.0, 1.5 Hz, H-2), 4.02 (1H,
dd, J = 8.8, 1.4 Hz, H-4), 4.08 (1H, dd, J = 8.8, 1.5 Hz, H-3), 4.15 (1H, ddd, J =
7.2, 5.8, 1.4 Hz, H-6), 4.48/4.55 (each 1H, d, J = 11.8 Hz, PhCH₂), 4.60/4.62 (each 1H,
d, J = 6.5 Hz, OCH₂OCH₃), 4.69/4.746 (each 1H, d, J = 6.5 Hz,
OCH₂OCH₃), 4.71/4.75 (each 1H, d, J = 6.5 Hz, OCH₂OCH₃), 4.742/4.82 (each 1H, d,
J = 12.0 Hz, PhCH₂), 7.22-7.35 (10H, m, arom.).
13j: ¹H NMR (CDCl₃) (chemical shift): 1.45/1.46 [each 3H, s, (CH₃)₂C], 3.30/3.35/
3.40 (each 3H, s, OCH₂OCH₃), 3.57 (1H, dd, J = 9.2, 5.8 Hz, H-7a), 3.58 (1H, dd, J =
10.1, 5.8 Hz, H-1a), 3.72 (1H, dd, J = 9.2, 7.5 Hz, H-7b), 3.73 (1H, dd, J = 1.5, 1.5 Hz,
H-5), 3.74 (1H, dd, J = 10.1, 6.3 Hz, H-1b), 3.82 (1H, ddd, J = 6.3, 5.8, 1.4 Hz, H-2),
3.99 (1H, dd, J = 8.6, 1.4 Hz, H-3), 4.12 (1H, ddd, J = 7.5, 5.8, 1.5 Hz, H-6), 4.22 (1H,
dd, J = 8.6, 1.5 Hz, H-4), 4.51/4.56 (each 1H, d, J = 11.7 Hz, PhCH₂), 4.54/4.56 (each
1H, d, J = 6.9 Hz, OCH₂OCH₃), 4.60/4.87 (each 1H, d, J = 7.0 Hz, OCH₂OCH₃),
4.66/4.76 (each 1H, d, J = 11.7 Hz, PhCH₂), 4.67/4.90 (each 1H, d, J = 6.6 Hz,
OCH₂OCH₃), 7.23-7.36 (10H, m, arom).
13i: ¹³C NMR (CDCl₃) (chemical shift): 19.0/29.6 [(CH₃)₂C], 55.5/55.7/
56.1 (OCH₂OCH₃), 67.5 (C-1), 69.5 (C-7), 69.6 (C-5), 71.6 (C-4), 72.4 (C-6), 73.1/
73.4 (PhCH₂), 76.2 (C-2), 77.2 (C-3), 96.9/98.3/98.6 (OCH₂OCH₃), 99.0 [(CH₃)₂C],
127.2/127.7/127.8/128.2/128.4 (d, arom.), 138.0/139.1 (s, arom.).
13j: ¹³C NMR (CDCl₃) (chemical shift): 19.1/29.6 [(CH₃)₂C], 55.3/56.1/
56.3 (OCH₂OCH₃), 68.2 (C-1), 69.1 (C-7), 69.6 (C-5), 72.0 (C-6), 73.5/73.7 (PhCH₂),
74.0 (C-4), 74.6 (C-2), 76.5 (C-3), 96.67/96.72/98.7 (OCH₂OCH₃), 99.0 [(CH₃)₂C],
127.5/127.7/127.75/127.82/128.2/128.4 (d, arom), 137.9/138.6 (s, arom).
13i: FABMS m/z: 565 [M + H]⁺ (pos.), FABHRMS m/z: 565.3043 (C₃₀H₄₅O₁₀ requires
565.3013).
13j: FABMS m/z: 565 [M + H]⁺ (pos.), FABHRMS m/z: 565.2983 (C₃₀H₄₅O₁₀ requires
565.3013).

### Example 22

The compound 16g (2.93 g, 5.20 mmol) obtained by Example 21 was treated in substantially the same manner as in Example 6 to give 4,6-O-isopropylidene-1,2,3-tri-O-methoxymethyl-D-glycero-L-galacto-heptitol (17g) (1.91 g; yield, 96%). The results of measurement for specific rotatory power, infrared absorption spectrum, ¹H-NMR spectrum, ¹³C-NMR spectrum as well as mass analysis FAB (Fast Atom Bombartmemt)-MS and HR-FAB-MS of the resulting compound (17g) are indicated as below:

TABLE 43
17g: colorless oil, Bp. 180-182° C./0.03 mmHg. [α]_{D}²⁴ −10.2 (c = 1.16, CHCl₃). IR
(neat): 3460, 1458, 1384, 1265, 1203, 1158, 1108, 1061, 1029 cm⁻¹.
17g: ¹H NMR (500 MHz, CDCl₃): 1.456/1.463 [each 3H, s, (CH₃)₂C], 2.34 (1H, dd, J = 8.0,
3.6 Hz, OH), 3.24 (1H, d, J = 8.6 Hz, OH), 3.38/3.39/3.45 (each 3H, s, OCH₂OCH₃),
3.68 (1H, dd, J = 9.7, 8.3 Hz, H-1a), 3.71 (1H, ddd, J = 8.6, 1.4, 1.2 Hz, H-5), 3.76 (1H,
dd, J = 9.7, 5.7 Hz, H-1b), 3.78 (1H, dd, J = 11.5, 8.0, 4.2 Hz, H-7a), 3.90, (1H, dd, J = 11.5,
6.3, 3.6 Hz, H-7b), 3.93 (1H, dd, J = 9.2, 1.8 Hz, H-3), 3.95 (1H, ddd, J = 8.3, 5.7,
1.8 Hz, H-2), 3.98 (1H, ddd, J = 6.3, 4.2, 1.4 Hz, H-6), 4.01 (1H, dd, J = 9.2, 1.2 Hz,
H-4), 4.64/4.66 (each 1H, d, J = 6.6 Hz, OCH₂OCH₃), 4.71/4.72 (each 1H, d, J = 6.6 Hz,
OCH₂OCH₃), 4.79/4.82 (each 1H, d, J = 6.3 Hz, OCH₂OCH₃).
17g: ¹³C NMR (125 MHz, CDCl₃) δ: 19.1/29.6 [(CH₃)₂C], 55.6/55.8/56.5 (OCH₂OCH₃),
63.2 (C-5), 63.6 (C-7), 67.1 (C-1), 70.7 (C-4), 72.7 (C-6), 75.6 (C-2), 76.5 (C-3),
97.0/98.0/99.0 (OCH₂OCH₃), 99.4 [(CH₃)₂C].
17g: FABMS m/z: 385 [M + H]⁺ (pos.), FABHRMS m/z: 385.2071 (C₁₆H₃₃O₁₀ require
385.2074).

### Example 23

The compound 17g (740 mg, 1.93 mmol) obtained by Example 22 was treated in accordance with Example 7 to give 4,6-O-isopropylidene-1,2,3-tri-O-methoxymethyl-D-glycero-L-galacto-heptitol 5,7-cyclosulfate 2g (580 mg; yield, 68%). The results of measurement for melting point, specific rotatory power, infrared absorption spectrum, ¹H-NMR spectrum, ¹³C-NMR spectrum as well as mass analysis FAB (Fast Atom Bombartmemt)-MS and HR-FAB-MS of the resulting compound 2g are indicated as below:

TABLE 44
2g: Colorless prisms. Mp. 102-103° C. [α]_{D}²⁴ +9.1 (c = 1.76, CHCl₃). IR (nujol): 1196,
1157, 1107, 1034 cm⁻¹.
2g: ¹H NMR (CDCl₃) (chemical shift): 1.47/1.50 [each 3H, s, (CH₃)₂C], 3.38/3.39/
3.44 (each 3H, s, OCH₂OCH₃), 3.69 (1H, dd, J = 9.8, 8.6 Hz, H-1a), 3.78 (1H, dd, J = 9.8, 5.8 Hz,
H-1b), 3.93 (1H, ddd, J = 8.6, 5.8, 1.5 Hz, H-2), 3.95 (1H, ddd, J = 1.7, 1.5, 1.5 Hz,
H-6), 4.00 (1H, dd, J = 9.2, 1.5 Hz, H-3), 4.21 (1H, dd, J = 9.2, 1.5 Hz, H-4), 4.55 (1H,
dd, J = 12.3, 1.5 Hz, H-7a), 4.64/4.66/4.70/4.71 (each 1H, d, J = 6.6 Hz, OCH₂OCH₃),
4.75/4.77 (each 1H, d, J = 6.3 Hz, OCH₂OCH₃), 4.90 (1H, dd, J = 12.3, 1.7 Hz, H-7b),
4.98 (1H, dd, J = 1.5, 1.5 Hz, H-5).
2g: ¹³C NMR (CDCl₃) (chemical shift): 18.8/29.1 [(CH₃)₂C], 55.6/55.9/
56.6 (OCH₂OCH₃), 61.9 (C-6), 66.9 (C-1), 68.5 (C-4), 74.8 (C-3), 75.2 (C-7), 75.5 (C-2),
76.4 (C-5), 97.0/98.1/99.0 (OCH₂OCH₃), 99.7 [(CH₃)₂C].
2g: FABMS m/z: 445 [M − H]⁻ (neg.), FABHRMS m/z: 445.1387 (C₁₆H₂₉O₁₂S requires
415.1380).

### Example 24

A mixture of the compound (2a) (200 mg, 0.45 mmol) obtained by Example 7, 1,4-dideoxy-1,4-epithio-D-arabinitol (7) (51.7 mg, 0.35 mmol), potassium carbonate (15 mg, 0.11 mmol) and 1,1,1,3,3,3-hexafluoroisopropanol (HFIR, 0.5 ml) was stirred at 60° C. for 42 hours, yielding the hydroxy group-protected cyclic sulfonium salt (8a) (187 mg; yield, 91%).

The processes were followed by using each of the compounds (2b) (300 mg, 0.67 mmol), (2c) (130 mg, 0.29 mmol) and (2d) (73 mg, 0.16 mmol) to give the hydroxy group-protected cyclic sulfonium salt (8b) (278 mg; yield, 90%), the hydroxy group-protected cyclic sulfonium salt (8c) (135 mg; yield, 85%) and the hydroxy group-protected cyclic sulfonium salt (8d) (72 mg; yield, 81%), respectively.

The results of measurement for melting point, specific rotatory power, infrared absorption spectrum, ¹H-NMR spectrum, ¹³C-NMR spectrum as well as mass analysis FAB (Fast Atom Bombartmemt)-MS and HR-FAB-MS of the resulting compounds (8a), (8b), (8c) and (8d) are indicated respectively as below:

TABLE 45
8a: Colorless prisms. Mp. 160-161° C. [α]_{D}²² +17.7 (c = 0.84,
CH₃OH), IR (nujol): 3344, 1265, 1211, 1150, 1103, 1030 cm⁻¹.
8b: Colorless prisms. Mp. 153-154° C. [α]_{D}²⁴ +40.8 (c = 1.15,
CH₃OH). IR (nujol): 3420, 3329, 1261, 1207, 1149, 1103, 1038 cm⁻¹.
8c: Colorless amorphous. [α]_{D}²⁵ +29.7 (c = 4.20, CH₃OH).
IR (nujol): 3391, 1255, 1207, 1157, 1103, 1022 cm⁻¹.
8d: Colorless amorphous. [α]_{D}²⁶ −10.5 (c = 3.38, CH₃OH).
IR (nujol): 3383, 1211, 1150, 1103, 1022 cm⁻¹.

TABLE 46-1
8a: ¹H-NMR (CD₃OD) (chemical shift): 1.47/1.54 (each 3H, s, (CH₃)₂C), 3.36/3.39/
3.41 (each 3H, s, OCH₂OCH₃), 3.78 (1H, dd, J = 12.8, 3.8 Hz, H-1a), 3.81 (2H, d-like, J = ca.
4.5 Hz, H-7′a and H-7′b), 3.85 (1H, m, dd, J = 12.8, 1.8 Hz, H-1b), 3.94 (1H, dd, J = 10.6,
1.7 Hz, H-5a), 3.98 (1H, dd, J = 10.6, 4.6 Hz, H-5b), 3.98-4.02 (1H, m, H-4),
4.06 (1H, dd, J = 13.8, 4.9 Hz, H-1′a), 4.08 (1H, dd, J = 6.9, 1.5 Hz, H-5′), 4.14 (1H, dd, J = 13.8,
3.2 Hz, H-1′b), 4.18 (1H, dd, J = 9.5, 1.5 Hz, H-4′), 4.24 (1H, dt-like, J = 6.9, 4.5 Hz,
H-6′), 4.40 (1H, ddd, J = 9.5, 4.9, 3.2 Hz, H-2′), 4.43 (1H, br d, J = 2.0, H-3),
4.50 (1H, dd, J = 9.5, 9.5 Hz, H-3′), 4.60-4.63 (1H, br m, H-2), 4.63/4.65 (each 1H, d, J = 6.3 Hz,
OCH₂OCH₃), 4.70/4.76/
4.89 (each 1H, d, J = 6.6 Hz, OCH₂OCH₃, a signal due to one of the methylene protons in
MOM groups overlapped with that of CD₃OH).
8b: ¹H NMR (CD₃OD) (chemical shift): 1.45/1.55 (each 3H s, (CH₃)₂C),
3.35/3.400/3.402 (each 3H, s, OCH₂OCH₃), 3.55 (1H, dd, J = 11.5, 7.8 Hz, H-7′a),
3.78 (1H, dd, J = 12.9, 3.8 Hz, H-1a), 3.83 (1H, dd, J = 12.9, 2.3 Hz, H-1b), 3.88 (1H, dd, J = 11.5,
1.5 Hz, H-7′b), 3.94 (1H, br dd, J = 7.2, 6.6 Hz, H-4 and 1H, dd, J = 8.0, 6.6 Hz,
H-5a), 3.99 (1H, dd, J = 8.0, 7.2 Hz, H-5b), 4.02 (1H, dd, J = 13.8, 4.6 Hz, H-1′a),
4.12 (1H, dd, J = 10.6, 13.8, 3.2 Hz, H-1′b), 4.15-4.20 (3H, m, H-4′, H-5′, H-6′), 4.35 (1H, dd,
J = 9.5, 9.5 Hz, H-3′), 4.40 (1H, ddd, J = 9.5, 4.6, 3.2 Hz, H-2′), 4.44 (1H, br d, J = 1.8,
H-3), 4.59-4.61 (1H, m, H-2), 4.60/4.63 (each 1H, d, J = 6.6 Hz, OCH₂OCH₃), 4.68/
4.77 (each 1H, d, J = 6.6 Hz, OCH₂OCH₃), 4.74/5.01 (each 1H, d, J = 6.9 Hz, OCH₂OCH₃).

TABLE 46-2
8c: ¹H NMR (CD₃OD) (chemical shift): 1.46/1.55 (each 3H, s, (CH₃)₂C), 3.39/3.42/
3.46 (each 3H, s, OCH₂OCH₃), 3.72 (1H, dd, J = 10.9, 5.4 Hz, H-7′a), 3.77-3.82 (2H, m, H-6′,
including one-proton doublet of doublets due to H-1a at δ 3.79 (J = 12.6, 3.7 Hz)],
3.84 (1H, dd, J = 12.6, 1.6 Hz, H-Ib), 3.90 (1H, dd, J = 10.9, 2.3 Hz, H-7b′), 3.93-3.99 (2H, m,
H-4 and H-5a), 4.00 (1H, dd, J = 10.4, 4.0 Hz, H-5b), 4.04 (1H, dd, J = 13.5, 5.2 Hz,
H-1a′), 4.11 (1H, dd, J = 9.5, 0.9 Hz, H-4′), 4.15 (1H, dd, J = 13.5, 2.9 Hz, H-1b′),
4.18 (1H, dd, J = 6.6, 0.9 Hz, H-5′), 4.41 (IH, dm, J = ca. 9.5, 0.9 Hz, H-4′), 4.43 (IH, br d, J = 2.3 Hz,
H-3), 4.4.8 (1H, dd, J = 9.5, 9.5 Hz, H-3′), 4.60-4.62 (1H, m, H-2), 4.62/
4.64 (each 1H, d, J = 6.6 Hz, OCH₂OCH₃), 4.70/4.72 (each 1H, d, J = 6.6 Hz, OCH₂OCH₃),
4.88/4.90 (each 1H, d, J = 6.3 Hz, OCH₂OCH₃).
8d: ¹H NMR (CD₃OD) (chemical shift): 1.47/1.55 (each 3H, s, (CH₃)₂C), 3.35/3.40/
3.42 (each 3H, s, OCH₂OCH₃), 3.65 (1H, dd, J = 11.0, 6.0 Hz, H-7a′), 3.77 (1H, dd, J = 12.7,
3.6 Hz, H-1a), 3.84 (1H, dd, J = 12.7, 1.6 Hz, H-Ib), 3.94 (1H, dd, J = 8.1, 4.9 Hz, H-5a),
3.97 (1H, dd, J = 11.0, 1.9 Hz, H-7b′), 3.97-3.99 (1H, m, H-4), 3.99 (1H, dd, J = 8.1, 3.6 Hz,
H-5b), 4.06 (1H, dd, J = 13.8, 4.6 Hz, H-1a′), 4.08 (1H, ddd, J = 6.4, 6.0, 1.9 Hz,
H-6′), 4.13 (1H, dd, J = 13.8, 3.2 Hz, H-1Ib′), 4.18 (1H, dd, J = 6.4, 1.2, Hz, H-5′),
4.24 (1H, dd, J = 9.6, 1.2 Hz, H-4′), 4.38 (1H, ddd, J = 9.6, 4.6, 3.2 Hz, H-2′), 4.43 (1H, d-like,
J = 2.2 Hz, H-3), 4.48 (1H, dd, J = 9.6, 9.6 Hz, H-3′), 4.60-4.63 (1H, m, H-2), 4.62/
4.67 (each 1H, d, J = 6.5 Hz, OCH₂OCH₃), 4.70/4.92 (each 1H, d. J = 6.3 Hz, OCH₂OCH₃),
4.74/4.75 (each 1H, d, J = 6.7 Hz, OCH₂OCH₃).

TABLE 47
8a: ¹³C-NMR (CD₃OD) (chemical shift): 19.3/29.2 [(CH₃)₂C], 50.5 (C-1′), 51.4 (C-1),
55.7/56.1/56.4 (OCH₂OCH₃), 60.9 (C-5), 69.9 (C-7′), 70.8 (C-3′), 71.3 (C-2′), 73.4 (C-4),
74.5 (C-4′), 78.7 (C-6′), 78.8 (C-5′), 79.2 (C-2), 80.1 (C-3), 97.8/98.7/98.8 (OCH₂OCH₃),
101.1 [(CH₃)₂C].
8b: ¹³C NMR (CD₃OD) (chemical shift): 19.3/29.1 [(CH₃)₂C], 50.5 (C-1′), 51.3 (C-1),
55.6/56.2/56.5 (OCH₂OCH₃), 60.9 (C-5), 69.7 (C-3′), 69.9 (C-7′), 70.9 (C-4′), 71.4 (C-2′),
73.5 (C-4), 77.5 (C-6′), 79.1 (C-2), 79.6 (C-5′), 80.1 (C-3), 97.6/98.3/100.7 (OCH₂OCH₃),
101.0 [(CH₃)₂C].
8c: ¹³C NMR (CD₃OD) (chemical shift): 19.3/29.1 [(CH₃)₂C], 50.6 (C-1′), 51.3 (C-1),
55.7/56.2/56.6 (OCH₂OCH₃), 60.9 (C-5), 68.9 (C-7′), 70.3 (C-3′), 71.2 (C-2′), 72.3 (C-4′),
73.5 (C-4), 77.0 (C-5′), 78.6 (C-6′), 79.2 (C-2), 80.0 (C-3), 97.8/98.0/100.3 (OCH₂OCH₃),
101.1 [(CH₃)₂C].
8d: ¹³C NMR (CD₃OD) (chemical shift): 19.2/29.2 [(CH₃)₂C], 50.5 (C-1′), 51.4 (C-1),
55.6/56.3/56.5 (OCH₂OCH₃), 60.9 (C-5), 69.7 (C-7′), 70.9 (C-3′), 71.4 (C-2′), 73.5 (C-4),
74.8 (C-4′), 77.8 (C-6′), 78.0 (C-5′), 79.2 (C-2), 80.1 (C-3), 97.8/97.9/98.7 (OCH₂OCH₃),
101.1 [(CH₃)₂C].

TABLE 48

8a: FABMS m/z: 597 [M + H]⁺ (pos.), FABHRMS
m/z: 597.1863 (C₂₁H₄₁O₁₅S₂ requires 597.1887).
8b: FABMS m/z: 597 [M + H]⁺ (pos.), FABHRMS
m/z: 597.1861 (C₂₁H₄₁O₁₅S₂ requires 597.1887).
8c: FABMS m/z: 597 [M + H]⁺ (pos.), FABHRMS
m/z: 597.1912 (C₂₁H₄₁O₁₅₂S₂ requires 597.1887).
8d: FABMS m/z: 597 [M + H]⁺ (pos.), FABHRMS
m/z: 597.1890 (C₂₁H₄₁O₁₅S₂ requires 597.1887).

### Example 25

A mixture of the compounds (8a) (158 mg) obtained by Example 24 and 30% trifluoroacetic acid aqueous solution (15 ml) was stirred at 50° C. for 2 hours to give the cyclic sulfonium salt (6a) in the amount of 88 mg (yield, 75%).

Each of the compounds (8b) (112 mg, 0.19 mmol), (8c) (78 mg, 0.134 mmol), and (8d) (41 mg, 0.071 mmol) was treated in substantially the same manner as above, thereby yielding the cyclic sulfonium salts (6b) (65.3 mg; yield, 85%), (6c) (53 mg; yield, 85%), and (6d) (27 mg; yield, 90%), respectively.

The results of measurement for specific rotatory power and infrared absorption spectrum of the resulting compounds (6a), (6b), (6c) and (6d) are indicated respectively as below:

TABLE 49

6a: colorless viscous oil. [α]_{D}²⁴ +12.4 (c = 0.97, H₂0).
IR (neat): 3380, 1271, 1238, 1215, 1135, 1108, 1065, 1025 cm⁻¹.
6b: Colorless amorphous. [α]_{D}²⁴ −8.1 (c = 1.13, H₂0).
IR (nujol): 3390, 1260, 1235, 1205, 1162, 1107, 1060, 1016 cm⁻¹.
6c: Colorless solid. [α]_{D}²⁴ −9.6 (c = 2.64, H₂0).
IR (nujol): 3368, 1260, 1227, 1163, 1150, 1105, 1072 cm⁻¹.
6d: Colorless solid. [α]_{D}²⁴ +4.4 (c = 2.31, H₂0).
1R (nujol): 3391, 1262, 1215, 1108, 1061 cm⁻¹.

The results of measurement for ¹H-NMR spectrum of the resulting compounds (6a), (6b), (6c) and (6d) are indicated respectively as below:

TABLE 50-1
6a: ¹H-NMR (CD₃OD) (chemical shift): 3.62 (1H, dd, J = 10.5, 6.5 Hz, H-7′a), 3.64 (1H,
dd, J = 10.6, 5.8 Hz, H-7′b), 3.76 (1H, dd, J = 8.3, 2.0 Hz, H-5′), 3.84 (1H, d-like, J = ca.
2.6 Hz, H-1a and H-1b), 3.88-3.93 (2H, m, H-1′a, including one-proton doublet of
doublets due to H-6′ at δ 3.91 (J = 6.5, 5.8 Hz), 3.93 (1H, dd, J = 8.6, 5.7 Hz, H-5a),
3.95-4.00 (2H, m, H-4, including one-proton doublet of doublets due to H-1′b at δ
3.99 (J = 13.5, 4.0 Hz), 4.03 (1H, dd, J = 8.6, 3.5 Hz, H-5b), 4.16 (1H, dd, J = 8.3, 2.6, H-4′),
4.38 (1H, d-like, J = ca. 2.6 Hz, H-3), 4.54 (1H, dd, J = 6.3, 6.3, 4.0 Hz, H-2′), 4.59 (1H,
dt-like, J = ca. 2.6, 2.6 Hz, H-2), 4.73 (1H, dd, J = 6.3, 2.6 Hz, H-3′).
6b: ¹H NMR (CD₃OD) (chemical shift): 3.61 (1H, dd, J = 11.2, 6.2 Hz, H-7′a), 3.68 (1H,
dd, J = 11.2, 4.5 Hz, H-7′b), 3.79 (1H, dd, J = 6.2, 4.6, 4.5 Hz, H-6′), 3.84 (2H, d-like, J = ca.
2.6 Hz, H-1a and H-1b), 3.89 (1H, dd, J = 4.6, 1.7 Hz, H-5′), 3.90-3.99 (3H, m,
H-4, including one-proton doublet of doublets due to H-1′a at δ 3.92 (J = 13.5, 3.7 Hz)
and one-proton doublet of doublets due to H-5 at δ 3.94 (J = 10.0, 8.0 Hz), 3.98 (1H,
dd, J = 13.5, 8.1 Hz, H-1′b), 3.99 (1H, dd, J = 6.9, 1.7 Hz, H-4′), 4.03 (1H, dd, J = 10.0,
4.9 Hz, H-5b), 4.38 (1H, dd, J = 2.6, 1.5 Hz, H-3), 4.47 (1H, dd, J = 6.9, 4.6 Hz, H-3′),
4.53 (1H, dd, J = 8.1, 4.6, 3.7 Hz, H-2′), 4.60 (1H, dt-like, J = ca. 2.6 Hz, H-2).

TABLE 50-2
2c: ¹H NMR (CD₃OD) (chemical shift): 3.63 (1H, dd, J = 11.0, 6.0 Hz, H-7′a), 3.67 (1H,
ddd, J = 8.6, 6.0, 3.0 Hz, H-6′), 3.78 (1H, dd, J = 8.6, 0.9 Hz, H-5′), 3.80 (1H, dd, J = 11.0,
3.0 Hz, H-7b′), 3.84 (2H, d-like, J = ca. 2.6 Hz, H-Ia and H-Ib), 3.89 (1H, dd, J = 13.3,
3.5 Hz, H-1a′), 3.92 (1H, dd, J = 10.7, 8.5 Hz, H-5a), 3.96 (1H, dd, J = 13.3, 7.7 Hz,
H-1′b), 3.97-4.01 (1H, m, H-4), 4.03 (1H, dd, J = 10.7, 5.2 Hz, H-5b), 4.11 (1H, dd, J = 7.8,
0.9 Hz, H-4′), 4.38 (1H, dd-like, J = ca. 2.6, 1.4 Hz, H-3), 4.45 (1H, dd, J = 7.8, 4.2 Hz,
H-3′), 4.54 (1H, ddd, J = 7.7, 4.2, 3.5 Hz, H-2′), 4.60 (1H, dt-like, J = ca. 2.6, 2.6 Hz,
H-2).
2d: ¹H NMR (CD₃OD) (chemical shift): 3.65 (1H, dd, J = 11.2, 5.2 Hz, H-7′a), 3.77 (1H,
dd, J = 11.2, 3.2 Hz, H-7b′), 3.77-3.82 (2H, m, H-5′ and H-6′), 3.84 (2H, d-like, J = ca.
2.6 Hz, H-1a and H-1b), 3.92 (1H, dd, J = 13.5, 6.9 Hz, H-1a′), 3.95 (1H, dd, J = 9.7, 7.5 Hz,
H-5a), 3.97-4.03 [2H, m, H-4, including one-proton doublet of doublets due to H-1′b
at δ ca. 4.00 (J = ca. 13.5, 3.8 Hz), 4.03 (1H, dd, J = 9.7, 4.9 Hz, H-5b), 4.23 (1H, dd,
J = 6.0, 2.9 Hz, H-4′), 4.39 (1H, dd, J = 2.6, 1.2 Hz, H-3), 4.54 (1H, ddd, J = 6.9, 6.0, 3.8 Hz,
H-2′), 4.60 (1H, dt, J = ca. 2.6, 2.6 Hz, H-2), 4.72 (1H, dd, J = 6.0, 2.9 Hz, H-3′).

The results of measurement for ¹³C-NMR spectrum of the resulting compounds (6a), (6b), (6c) and (6d) are indicated respectively as below:

TABLE 51
6a: ¹³C NMR (CD₃OD) (chemical shift): 51.5 (C-1), 52.7 (C-1′),
61.0 (C-5), 64.7 (C-7′), 68.0 (C-2′), 71.8 (C-6′), 72.2 (C-5′), 72.6 (C-4′),
73.3 (C-4), 79.2 (C-2), 79.7 (C-3), 81.9 (C-3′).
6b: ¹³C NMR (CD₃OD) (chemical shift): 51.7 (C-1 and C-1′),
60.9 (C-5), 64.0 (C-7′), 69.3 (C.2′), 70.8 (C-5′), 73.1 (C-4), 73.2 (C-4′),
74.8 (C-6′), 79.3 (C-2), 79.6 (C-3), 80.2 (C-3′).
6c: ¹³C NMR (CD₃OD) (chemical shift): 51.6 (C-1′), 51.7 (C-1),
60.9 (C-5), 65.1 (C-7′), 69.7 (C-2′), 70.9 (C-4′), 71.2 (C-5′), 72.4 (C-6′),
73.2 (C-4), 79.3 (C-2), 79.6 (C-3), 79.9 (C-3′).
6d: ¹³C NMR (CD₃OD) (chemical shift): 51.5 (C-1), 52.5 (C-1′),
61.0 (C-5), 64.4 (C-7′), 67.9 (C-2′), 73.3 (C-4), 73.8 (C-5′), 73.9 (C-4′),
74.3 (C-6′), 79.2 (C-2), 79.7 (C-3), 81.0 (C-3′).

The results of measurement for mass analysis FAB (Fast Atom Bombartmemt)-MS and HR-FAB-MS of the resulting compounds (6a), (6b), (6c) and (6d) are indicated respectively as below:

TABLE 52

6a: FABMS m/z: 425 [M + H]⁺ (pos.), FABHRMS
m/z: 425.0795 (C₁₂H₂₅O₁₂S₂ requires 425.0788).
6b: FABMS m/z: 425 [M + H]⁺ (pos.), FABHRMS
m/z: 425.0809 (C₁₂H₂₅O₁₂S₂ requires 425.0788).
6c: FABMS m/z: 425 [M + H]⁺ (pos.), FABHRMS
m/z: 425.0760 (C₁₂H₂₅O₁₂S₂ requires 425.0788).
6d: FABMS m/z: 425 [M + H]⁺ (pos.), FABHRMS
m/z: 425.0760 (C₁₂H₂₅O₁₂S₂ requires 425.0788).

### Example 26

The compound 2g (300 mg, 0.673 mmol) obtained by Example 23 was treated in accordance with Example 24 to form the hydroxy group-protected cyclic sulfonium salt (8g) in the amount of 107 mg (yield, 53%). The results of measurement for specific rotatory power, infrared absorption spectrum, ¹H-NMR spectrum, ¹³C-NMR spectrum as well as mass analysis FAB (Fast Atom Bombartmemt)-MS and HR-FAB-MS of the resulting compound (8g) are indicated as below:

8g: Colorless amorphous. [α]_{D}²⁴ −25.0 (c=1.17, CH₃OH). IR (nujol): 3364, 1262, 1207, 1153, 1107, 1026 cm⁻¹.

8g: ¹H NMR (CD₃OD) (chemical shift): 1.45/1.52 [each 3H, s, (CH₃)₂C], 3.35/3.37/3.39 (each 3H, s, OCH₂OCH₃), 3.76 (1H, dd, J=9.8, 6.7 Hz, H-7′a), 3.77-3.89 (3H, m, H-1a, H-1b and H-7′b), 3.88 (1H, dd, J=13.4, 3.6 Hz, H-1′a), 3.91 (1H, dd, J=11.0, 8.6 Hz, H-5a), 3.94 (1H, br t-like, J=6.7 Hz, H-6′), 3.97-4.00 (1H, m, H-4), 4.01 (1H, dd, J=13.4, 7.9 Hz, H-1′b), 4.03 (1H, dd, J=11.0, 5.5 Hz, H-5b), 4.07 (2H, br s-like, H-4′ and H-5′), 4.39 (1H, br dd-like, J=ca. 1.5 Hz, H-3), 4.54-4.58 (2H, m, H-2′ including br s-like signal due to H-3′ at δ 4.56), 4.59-4.61 (1H, m, H-2), 4.61/4.63 (each 1H, d, J=6.5 Hz, OCH₂OCH₃), 4.67/4.71 (each 1H, d, J=6.5 Hz, OCH₂OCH₃), 4.79/4.92 (each 1H, d, J=6.7 Hz, OCH₂OCH₃).

8g: ¹³C NMR (CD₃OD) (chemical shift): 19.5/29.5 [(C1-13)2C], 50.2 (C-1′), 51.2 (C-1), 55.8/56.1/56.2 (OCH₂OCH₃), 60.9 (C-5), 68.8 (C-7′), 70.1/72.2 (C-2′ and C-3′), 72.6 (C-4′), 73.4 (C-4), 77.4 (C-6′), 78.0 (C-5′), 79.2 (C-2), 79.8 (C-3), 97.9/99.1/100.4 (OCH₂CH₃), 101.2 [(CH₃)₂C].

8g: FABMS m/z: 597 [M+H]⁺ (pos.), FABHRMS m/z: 597.1865 (C₂₁H₄₁O₁₅S₂ requires 597.1887).

### Example 27

The compound 8g (48.6 mg, 0.082 mmol) obtained by Example 26 was treated in accordance with Example 25 to give the cyclic sulfonium salt (6g) in the amount of 31 mg (yield, 93%). The results of measurement for specific rotatory power, infrared absorption spectrum, ¹H-NMR spectrum, ¹³C-NMR spectrum as well as mass analysis FAB (Fast Atom Bombartmemt)-MS and HR-FAB-MS of the resulting compound (6g) are indicated as below:

6g: Colorless solid [α]_{D}²⁴ −27.3 (c=1.06, H₂O). IR (nujol): 3348, 1257, 1219, 1072 cm⁻¹.

6g: ¹H NMR (D₂O) (chemical shift): 3.64 (1H, dd, J=10.3, 7.2 Hz, H-7′a), 3.66 (1H, dd, J=10.3, 4.6 Hz, H-7′b), 3.73 (1H, dd, J=9.5, 1.4 Hz, H-5′), 3.83 (1H, dd, J=13.0, 3.2 Hz, H-1a), 3.85 (1H, dd, J=13.0, 2.2 Hz, H-1b), 3.89-3.93 (2H, m, H-5a and H-6′), 3.93-3.96 (2H, m, H-1′a and H-1′b), 4.00 (1H, br dd, J=8.9, 5.2 Hz, H-4), 4.04 (1H, dd, J=10.8, 5.2 Hz, H-5b), 4.09 (1H, dd, J=9.5, 1.2 Hz, H-4′), 4.37 (1H, dd-like, J=ca. 2.2, 1.2 Hz, H-3), 4.57-4.61 (1H, m H-2′), 4.62 (1H, dt-like, J=ca. 3.2, 2.2 Hz, H-2), 4.70 (1H, dd, J=5.1, 1.2 Hz, H-3′).

6g: ¹³C NMR (D₂O) (chemical shift): 51.1 (C-1′), 51.4 (C-1), 60.9 (C-5), 65.0 (C-7′), 69.1 (C-2′), 70.1 (C-4′), 70.7 (C-5′), 71.4 (C-6′), 73.4 (C-4), 78.7 (C-3′), 79.4 (C-2), 79.6 (C-3).

6g: FABMS m/z: 423 [M−H]⁻ (Neg.), FABHRMS m/z: 423.0617 (C₁₂H₂₃O₁₂S₂ requires 425.0788).

It was found from the data of ¹H-NMR and ¹³C-NMR spectra that, although they did not correspond with kotalanol (1), this compound is one of diastereomers of kotalanol.

### Example 28

The compounds (6a), (6b), (6c) and (6d), each prepared by Example 25, as well as the compound (6g) prepared by Example 27 were each measured for α-glycosidase-inhibiting activity.

An assay was carried out by suspending small intestinal brush border membrane vesicles in 0.1 M maleic acid buffer (pH 6.0) and using this resulting suspension as α-glycosidase (sucrase, maltase and isomaltase).

To each of sucrose (74 mM), maltose (74 mM) and isomaltose (74 mM) solution as a substrate, there was added a solution (0.05 ml) of a test compound in various concentrations, and the resulting mixture was pre-heated at 37° C. for 2 to 3 minutes, followed by addition of an enzyme solution and reaction for 30 minutes. Thereafter, water was added and the resulting mixture was heated in a boiling water bath for 2 minutes in order to inactivate the enzyme. Separately, the enzyme solution was added to each of the test solutions and immediately thereafter heated in boiling water bath for 2 minutes to inactivate the enzyme. This solution was used as a blank. The amount of purified D-glucose was measured in accordance with glucose-oxidase method. The substrate and test compounds were used in a solution of 0.1 M maleic acid buffer (pH 6.0). A 50% inhibitory concentration (IC₅₀) was calculated from the value observed.

TABLE 53

| | IC₅₀, (μg/ml) | | |
|---|---|---|---|
| Test Compounds | sucrase | maltase | isomaltase |
| kotalanol | 0.32 | 3.07 | 2.41 |
| Compound (6a) | 28.4 | 20.6 | 0.69 |
| Compound (6b) | 57.8 | >100 | 4.6 |
| Compound (6c) | 90.8 | >100 | 6.88 |
| Compound (6d) | 13.7 | 24.8 | 2.77 |
| Compound (6g) | >100 | >100 | 66.9 |

### INDUSTRIAL APPLICABILITY

The present invention provides an artificial synthesis of kotalanol analogues from starting materials ready to make available. Further, the synthesis routes of the present invention can make kotalanol that can be obtained from the nature in a very small amount. In addition, the kotalanol analogues according to the present invention have glycosidase inhibiting activity.

1. A cyclic sulfonium salt represented by general formula (1):

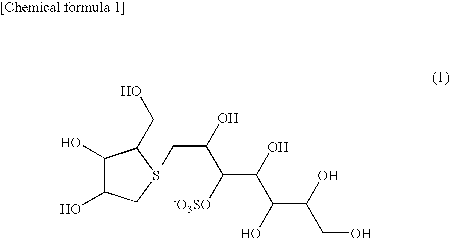

2. A method for the production of a cyclic sulfonium salt, comprising a step for esterifying a pentose selected from D-xylose, D-ribose, D-arabinose, D-lyxose, L-ribose, L-arabinose and L-lyxose and a derivative thereof to form a cyclic sulfate ester of a heptitol with a protected hydroxy group, as represented by general formula (2): 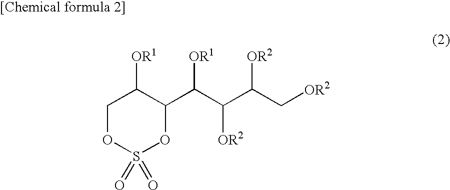
(wherein R¹ and R² are each a hydrogen atom or a hydroxy group-protective group, in which the hydroxy group-protective group comprises a cyclic acetal-protective group selected from —C(CH₃)₂—, —CH(CH₃)— and —CHAr— (wherein Ar is a phenyl group or a substituted phenyl group), an ether-type protective group comprising an alkoxyalkyl group as represented by —CH₂OR³ (wherein R³ is —CH₂OCH₃ or —CH₂CH₂OCH₃) or a silyl ether-type protective group as represented by SiR⁴₃ or SiR⁴₂R⁵ (wherein R⁴ and R⁵ are each an alkyl group as represented by —CH₃ or —C(CH₃)₃ or an aryl group as represented by —Ph);
a coupling step for coupling the resulting cyclic sulfate ester of the heptitol (2) with a thiosugar as represented by general formula (7′): 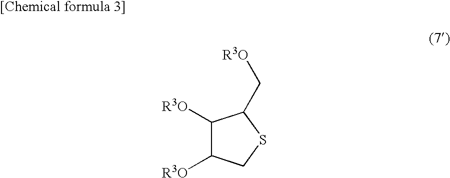
(wherein R³ is hydrogen atom or a hydroxy group-protective group comprising a cyclic acetal-protective group selected from −C(CH₃)₂—, —CH(CH₃)— and —CHAr— (wherein Ar is a phenyl group or a substituted phenyl group), an ether-type protective group comprising an alkoxyalkyl group as represented by —CH₂OR³ (wherein R³ is —CH₂OCH₃ or —CH₂CH₂OCH₃) or a silyl ether-type protective group as represented by SiR⁴₃ or SiR⁴₂R⁵ (wherein R⁴ and R⁵ are each an alkyl group as represented by —CH₃ or —C(CH₃)₃ or an aryl group as represented by —Ph)
to yield a cyclic sulfonium salt with the protected hydroxy group as represented by general formula (8′): 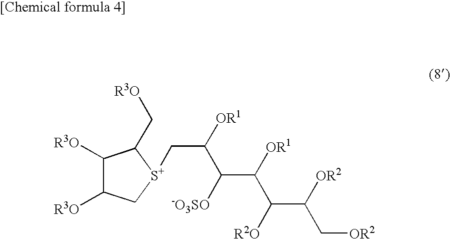
and a step for deprotecting the hydroxy group-protective group of the hydroxy group-protected cyclic sulfonium salt to yield a cyclic sulfonium salt as represented by general formula (1)

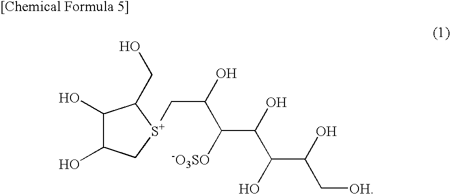

3. The method for the production of the cyclic sulfonium salt as claimed in claim 2, wherein the thiosugar (7′) to be used for said coupling step is synthesized from D-xylose.

4. A cyclic sulfate ester of a heptitol with the protected hydroxy group as represented by general formula (2): 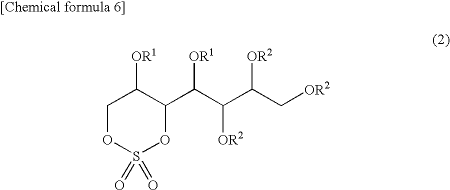
(wherein R¹ and R² are each a hydrogen atom or a protective group for hydroxy group, in which the protective group comprises a cyclic acetal-protective group selected from −C(CH₃)₂—, —CH(CH₃)— and —CHAr— (wherein Ar is a phenyl group or a substituted phenyl group), an ether-type protective group comprising an alkoxyalkyl group as represented by —CH₂OR³ (wherein R³ is —CH₂OCH₃ or —CH₂CH₂OCH₃) or a silyl ether-type protective group as represented by SiR⁴₃ or SiR⁴₂R⁵ (wherein R⁴ and R⁵ are each an alkyl group as represented by —CH₃ or —C(CH₃)₃ or an aryl group as represented by —Ph):

5. A method for the production of a cyclic sulfate ester of a heptitol with the protected hydroxy group, wherein a pentose selected from D-xylose, D-ribose, D-arabinose, D-lyxose, L-xylose, L-ribose, L-arabinose and L-Iyxose and a derivative thereof, as represented by general formula (3) or (4), are reacted to form a cyclic sulfate ester of a heptitol (2) with the protected hydroxy group:

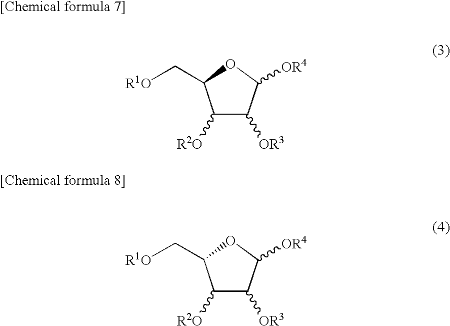

6. A glycosidase inhibitor containing the cyclic sulfonium salt as claimed in claim 1.

7. An anti-diabetic agent or an anti-diabetic food containing a glycosidase inhibitor as in claim 6.
